(19) United States
(12) Patent Application Publication
(10) Pub. No.: US 2022/0393045 A1
(43) Pub. Date: Dec. 8, 2022

(54) IMAGING ELEMENT, STACKED IMAGING ELEMENT, SOLID-STATE IMAGING DEVICE, AND INORGANIC OXIDE SEMICONDUCTOR MATERIAL

(71) Applicant: SONY GROUP CORPORATION, TOKYO (JP)

(72) Inventors: YOICHIRO IINO, TOKYO (JP); HIROSHI NAKANO, TOKYO (JP); TOSHIKI MORIWAKI, TOKYO (JP)

(21) Appl. No.: 17/760,060

(22) PCT Filed: Jan. 11, 2021
(86) PCT No.: PCT/JP2021/000591

§ 371 Date: Aug. 3, 2022

(30) Foreign Application Priority Data
Feb. 12, 2020 (JP) 2020-021439

(51) Int. Cl. H01L 31/032; H01L 31/109

(52) U.S. Cl. CPC H01L 31/032; H01L 31/109

(57) ABSTRACT

An imaging element according to the present disclosure includes: a photoelectric conversion unit that is configured of a first electrode 21 and a photoelectric conversion layer 23A and a second electrode 22 including an organic material being laminated, an inorganic oxide semiconductor material layer 23B is formed between the first electrode 21 and the photoelectric conversion layer 23A, and an inorganic oxide semiconductor material configuring the inorganic oxide semiconductor material layer 23B contains gallium (Ga) atoms, tin (Sn) atoms, zinc (Zn) atoms, and oxygen (O) atoms.

### TECHNICAL FIELD

The present disclosure relates to an imaging element, a stacked imaging element, a solid-state imaging device, and an inorganic oxide semiconductor material.

### BACKGROUND ART

As an imaging element configuring an image sensor or the like, a stacked imaging element has attracted attention in recent years. The stacked imaging element has a structure in which a photoelectric conversion layer (light receiving layer) is sandwiched by two electrodes. Also, the stacked imaging element requires a structure in which signal charge generated in the photoelectric conversion layer on the basis of photoelectric conversion is accumulated and transferred. The structure in the related art requires a structure in which signal charge is accumulated in a floating diffusion layer and is transferred and requires high-speed transfer such that no delay occurs in the signal charge.

An imaging element (photoelectric conversion element) to solve such a problem is disclosed in JP 2016-063165A, for example. The imaging element includes: an accumulation electrode that is formed on a first insulating layer;

a second insulating layer that is formed on the accumulation electrode;
a semiconductor layer that is formed to cover the accumulation electrode and the second insulating layer;
a collection electrode that is formed to come into contact with the semiconductor layer and to be separated from the accumulation electrode;
a photoelectric conversion layer that is formed on the semiconductor layer; and
an upper electrode that is formed on the photoelectric conversion layer.

An imaging element that uses an organic semiconductor material in a photoelectric conversion layer can perform photoelectric conversion on a specific color (wavelength band). In a case in which the imaging element is used in a solid-state imaging device, it is possible to obtain a structure in which sub-pixels are laminated (stacked imaging element), which cannot be achieved by a solid-state imaging device in the related art where a combination of an on-chip color filter (OCCF) and an imaging element forms each sub-pixel and the sub-pixels are two-dimensionally aligned, due to the aforementioned feature (see JP 2011-138927A, for example). Also, there is an advantage that no false colors occur since demosaic processing is not required. In the following description, an imaging element including a photoelectric conversion unit provided on or above a semiconductor substrate may be referred to as a “first type imaging element” for convenience, the photoelectric conversion unit configuring the first type imaging element may be referred to as a “first type photoelectric conversion unit” for convenience, the imaging element provided in the semiconductor substrate may be referred to as a “second type imaging element” for convenience, and a photoelectric conversion unit configuring the second type imaging element may be referred to as a “second type photoelectric conversion unit” for convenience.

FIG. 70 illustrates a configuration example of a stacked imaging element (stacked solid-state imaging device) in the related art. In the example illustrated in FIG. 70, a third photoelectric conversion unit 343A and a second photoelectric conversion unit 341A that are second type photoelectric conversion units configuring a third imaging element 343 and a second imaging element 341 that are second type imaging elements are laminated and formed in a semiconductor substrate 370. Also, a first photoelectric conversion unit 310A that is a first type photoelectric conversion unit is disposed above the semiconductor substrate 370 (specifically, above the second imaging element 341). Here, the first photoelectric conversion unit 310A includes a first electrode 321, and a photoelectric conversion layer 323 including an organic material and a second electrode 322 and configures a first imaging element 310 that is a first type imaging element. In the second photoelectric conversion unit 341A and the third photoelectric conversion unit 343A, photoelectric conversion of blue light and red light, for example, is performed, respectively, due to a difference in absorption coefficients. In the first photoelectric conversion unit 310A, photoelectric conversion of green light is performed, for example.

Charge generated through photoelectric conversion in the second photoelectric conversion unit 341A and the third photoelectric conversion unit 343A is temporarily accumulated in the second photoelectric conversion unit 341A and the third photoelectric conversion unit 343A, is then transferred to a second floating diffusion layer FD₂ and a third floating diffusion layer FD₃ by a vertical transistor (a gate portion 345 is illustrated) and a transfer transistor (a gate portion 346 is illustrated), respectively, and is further output to an external reading circuit (not illustrated). The transistors and the floating diffusion layers FD₂ and FD₃ are also formed in the semiconductor substrate 370.

Charge generated through photoelectric conversion in the first photoelectric conversion unit 310A is accumulated in a first floating diffusion layer FD₁ formed in the semiconductor substrate 370 via a contact hole portion 361 and a wiring layer 362. Also, the first photoelectric conversion unit 310A is also connected to a gate portion 352 of an amplification transistor, which converts the amount of charge into a voltage, via the contact hole portion 361 and the wiring layer 362. Also, the first floating diffusion layer FD₁ configures a part of a reset transistor (a gate portion 351 is illustrated). The reference sign 371 denotes an element separation region, the reference sign 372 denotes an oxide film formed on the surface of the semiconductor substrate 370, the reference signs 376 and 381 denote interlayer insulating layers, the reference sign 383 denotes a protective material layer, and the reference sign 314 denotes an on-chip micro lens.

### CITATION LIST

### Patent Literature

### [PTL 1]

JP 2016-063165A

### [PTL 2]

JP 2011-138927A

### SUMMARY

### Technical Problem

However, the technology disclosed in JP 2016-063165A described above has a limit that an accumulation electrode and a second insulating layer formed thereon have to be formed to have the same length and finely defines an interval from a collection electrode and the like, which may lead to a complicated production process and thus a decrease in manufacturing yield. Moreover, although some materials configuring a semiconductor layer are mentioned, there is no mention regarding more specific compositions and configurations of the materials. Also, a correlation expression between carrier mobility and accumulated charge in the semiconductor layer is mentioned. However, there is no mention concerning matters in relation to an improvement in properties of transferring the generated charge at all.

Therefore, an object of the present disclosure is to provide an imaging element, a stacked imaging element, a solid-state imaging device, and an inorganic oxide semiconductor material with excellent properties of transferring charge accumulated in a photoelectric conversion layer regardless of a simple configuration and structure.

### Solution to Problem

An imaging element according to the present disclosure to achieve the aforementioned object includes: a photoelectric conversion unit that is configured of a first electrode, a photoelectric conversion layer containing an organic material and a second electrode being laminated, an inorganic oxide semiconductor material layer being formed between the first electrode and the photoelectric conversion layer, and an inorganic oxide semiconductor material configuring the inorganic oxide semiconductor material layer containing gallium (Ga) atoms, tin (Sn) atoms, zinc (Zn) atoms, and oxygen (O) atoms.

A stacked imaging element according to the present disclosure to achieve the aforementioned object includes at least one imaging element according to the present disclosure described above.

A solid-state imaging device according to a first aspect of the present disclosure to achieve the aforementioned object includes a plurality of imaging elements according to the present disclosure described above. Also, a solid-state imaging device according to a second aspect of the present disclosure to achieve the aforementioned object includes a plurality of stacked imaging elements according to the present disclosure described above.

An inorganic oxide semiconductor material according to the present disclosure to achieve the aforementioned object has a composition represented as GaₐSn_{b}Zn_{c}O_{d} (where a+b+c=1.00, and a>0, b>0, and c>0), values of a, b, and c satisfying Expression (1) below, or satisfying Expression (2) below, or satisfying Expression (3) below, or satisfying Expressions (1) and (2) below, or satisfying Expressions (1) and (3) below, or satisfying Expressions (2) and (3) below, or satisfying Expressions (1), (2), and (3) below, here,

0.45(b−0.62)≤0.55a≤0.45b (1)

a≤−3.0(b−0.63) (2)

b≥0.23 (3)

Note that d=1.5a+2.0b+c is preferably satisfied.

Alternatively, it is desirable that b>a and b>c be satisfied.

### BRIEF DESCRIPTION OF DRAWINGS

FIG. 1 is a schematic partial sectional view of an imaging element in Example 1.

FIG. 2 is an equivalent circuit diagram of the imaging element in Example 1.

FIG. 3 is an equivalent circuit diagram of the imaging element in Example 1.

FIG. 4 is a schematic disposition diagram of a first electrode and a charge accumulation electrode configuring the imaging element in Example 1 and transistors configuring a control unit.

FIG. 5 is a diagram schematically illustrating a state of a potential at each portion when the imaging element in Example 1 is operating.

FIGS. 6A, 6B, and 6C are equivalent circuit diagrams of imaging elements in Examples 1, 4, and 6 for explaining each portion in FIG. 5 (Example 1), FIGS. 20 and 21 (Example 4), and FIGS. 32 and 33 (Example 6).

FIG. 7 is a schematic disposition diagram of the first electrode and the charge accumulation electrode configuring the imaging element in Example 1.

FIG. 8 is a schematic transparent perspective view of the first electrode, the charge accumulation electrode, a second electrode, and a contact hole portion configuring the imaging element in Example 1.

FIG. 9 is an equivalent circuit diagram of a modification example of the imaging element in Example 1.

FIG. 10 is a schematic disposition diagram of a first electrode and a charge accumulation electrode configuring a modification example of the imaging element in Example 1 illustrated in FIG. 9 and transistors configuring a control unit.

FIG. 11 is a schematic partial sectional view of an imaging element in Example 2.

FIG. 12 is a schematic partial sectional view of an imaging element in Example 3.

FIG. 13 is a schematic partial sectional view of a modification example of the imaging element in Example 3.

FIG. 14 is a schematic partial sectional view of another modification example of the imaging element in Example 3.

FIG. 15 is a schematic partial sectional view of yet another modification example of the imaging element in Example 3.

FIG. 16 is a schematic partial sectional view of a part of an imaging element in Example 4.

FIG. 17 is an equivalent circuit diagram of the imaging element in Example 4.

FIG. 18 is an equivalent circuit diagram of the imaging element in Example 4.

FIG. 19 is a schematic disposition diagram of a first electrode, a transfer control electrode, and a charge accumulation electrode configuring the imaging element in Example 4 and transistors configuring a control unit.

FIG. 20 is a diagram schematically illustrating a state of a potential at each portion when the imaging element in Example 4 is operating.

FIG. 21 is a diagram schematically illustrating a state of a potential at each portion when the imaging element in Example 4 is performing another operation.

FIG. 22 is a schematic disposition diagram of the first electrode, the transfer control electrode, and the charge accumulation electrode configuring the imaging element in Example 4.

FIG. 23 is a schematic transparent perspective view of the first electrode, the transfer control electrode, the charge accumulation electrode, a second electrode, and a contact hole portion configuring the imaging element in Example 4.

FIG. 24 is a schematic disposition diagram of a first electrode, a transfer control electrode, and a charge accumulation electrode configuring a modification example of the imaging element in Example 4 and transistors configuring a control unit.

FIG. 25 is a schematic partial sectional view of a part of an imaging element in Example 5.

FIG. 26 is a schematic disposition diagram of a first electrode, a charge accumulation electrode, and a charge discharging electrode configuring the imaging element in Example 5.

FIG. 27 is a schematic transparent perspective view of the first electrode, the charge accumulation electrode, the charge discharging electrode, a second electrode, and a contact hole portion configuring the imaging element in Example 5.

FIG. 28 is a schematic partial sectional view of an imaging element in Example 6.

FIG. 29 is an equivalent circuit diagram of the imaging element in Example 6.

FIG. 30 is an equivalent circuit diagram of the imaging element in Example 6.

FIG. 31 is a schematic disposition diagram of a first electrode and a charge accumulation electrode configuring the imaging element in Example 6 and transistors configuring a control unit.

FIG. 32 is a diagram schematically illustrating a state of a potential at each portion when the imaging element in Example 6 is operating.

FIG. 33 is a diagram schematically illustrating a state of a potential at each portion when the imaging element in Example 6 is performing another operation (transfer).

FIG. 34 is a schematic disposition diagram of the first electrode and the charge accumulation electrode configuring the imaging element in Example 6.

FIG. 35 is a schematic transparent perspective view of the first electrode, the charge accumulation electrode, a second electrode, and a contact hole portion configuring the imaging element in Example 6.

FIG. 36 is a schematic disposition diagram of a first electrode and a charge accumulation electrode configuring a modification example of the imaging element in Example 6.

FIG. 37 is a schematic sectional view of a part of imaging elements (two imaging elements aligned side by side) in Example 7.

FIG. 38 is a schematic disposition diagram of a first electrode, a charge accumulation electrode, and the like configuring the imaging element in Example 7 and transistors configuring a control unit.

FIG. 39 is a schematic disposition diagram of the first electrode, the charge accumulation electrode, and the like configuring the imaging element in Example 7.

FIG. 40 is a schematic disposition diagram of a modification example of the first electrode, the charge accumulation electrode, and the like configuring the imaging element in Example 7.

FIG. 41 is a schematic disposition diagram of a modification example of the first electrode, the charge accumulation electrode, and the like configuring the imaging element in Example 7.

FIGS. 42A and 42B are schematic disposition diagrams of a modification example of the first electrode, the charge accumulation electrode, and the like configuring the imaging element in Example 7.

FIG. 43 is a schematic sectional view of a part of imaging elements (two imaging elements aligned side by side) in Example 8.

FIG. 44 is a schematic plan view of a part of the imaging elements (2×2 imaging elements aligned side by side) in Example 8.

FIG. 45 is a schematic plan view of a part of a modification example of the imaging elements (2×2 imaging elements aligned side by side) in Example 8.

FIGS. 46A and 46B are schematic sectional views of a part of a modification example of the imaging elements (2 imaging elements aligned side by side) in Example 8.

FIGS. 47A and 47B are schematic sectional views of a part of a modification example of the imaging elements (2 imaging elements aligned side by side) in Example 8.

FIGS. 48A and 48B are schematic plan views of a part of a modification example of the imaging elements in Example 8.

FIGS. 49A and 49B are schematic plan views of a part of a modification example of the imaging elements in Example 8.

FIG. 50 is a schematic plan view of a first electrode and a charge accumulation electrode segment in a solid-state imaging device in Example 9.

FIG. 51 is a schematic plan view of a first electrode and a charge accumulation electrode segment in a first modification example of the solid-state imaging device in Example 9.

FIG. 52 is a schematic plan view of a first electrode and a charge accumulation electrode segment in a second modification example of the solid-state imaging device in Example 9.

FIG. 53 is a schematic plan view of a first electrode and a charge accumulation electrode segment in a third modification example of the solid-state imaging device in Example 9.

FIG. 54 is a schematic plan view of a first electrode and a charge accumulation electrode segment in a fourth modification example of the solid-state imaging device in Example 9.

FIG. 55 is a schematic plan view of a first electrode and a charge accumulation electrode segment in a fifth modification example of the solid-state imaging device in Example 9.

FIG. 56 is a schematic plan view of a first electrode and a charge accumulation electrode segment in a sixth modification example of the solid-state imaging device in Example 9.

FIG. 57 is a schematic plan view of a first electrode and a charge accumulation electrode segment in a seventh modification example of the solid-state imaging device in Example 9.

FIGS. 58A, 58B, and 58C are charts illustrating a reading drive example in an imaging element block in Example 9.

FIG. 59 is a schematic plan view of a first electrode and a charge accumulation electrode segment in a solid-state imaging device in Example 10.

FIG. 60 is a schematic plan view of a first electrode and a charge accumulation electrode segment in a modification example of the solid-state imaging device in Example 10.

FIG. 61 is a schematic plan view of a first electrode and a charge accumulation electrode segment in a modification example of the solid-state imaging device in Example 10.

FIG. 62 is a schematic plan view of a first electrode and a charge accumulation electrode segment in a modification example of the solid-state imaging device in Example 10.

FIG. 63 is a schematic partial sectional view of yet another modification example of the imaging element and the stacked imaging element in Example 1.

FIG. 64 is a schematic partial sectional view of yet another modification example of the imaging element and the stacked imaging element in Example 1.

FIG. 65 is a schematic partial sectional view of yet another modification example of the imaging element and the stacked imaging element in Example 1.

FIG. 66 is a schematic partial sectional view of another modification example of the imaging element and the stacked imaging element in Example 1.

FIG. 67 is a schematic partial sectional view of yet another modification example of the imaging element in Example 4.

FIG. 68 is a conceptual diagram of the solid-state imaging device in Example 1.

FIG. 69 is a conceptual diagram of an example in which the solid-state imaging device including the imaging element and the stacked imaging element according to the present disclosure is used as the electronic equipment (camera).

FIG. 70 is a conceptual diagram of a stacked imaging element (stacked solid-state imaging device) in the related art.

FIGS. 71A and 71B are a graph plotting a relationship between (a, b, c) values and an optical gap value in an inorganic oxide semiconductor material with a composition of GaₐSn_{b}Zn_{c}O_{d} and a graph plotting a relationship between (a, b, c) values and an oxygen deficiency generation energy value, respectively.

FIGS. 72A and 72B are a graph plotting a relationship between (a, b, c) values and a carrier mobility value in an inorganic oxide semiconductor material with a composition of GaₐSn_{b}Zn_{c}O_{d} and a graph plotting Examples 1A, 1B, and 1C and Comparative Examples 1A, 1B, and 1C on a graph illustrating a region that satisfies Expressions (1), (2), (2-3), (2-4), and (3) of the (a, b, c) values, respectively.

FIG. 73 is a diagram in which results of measuring a threshold voltage Vₜₕ are written on FIG. 72B in Example 1.

FIG. 74 is a diagram in which a result of measuring carrier mobility μ is written on FIG. 72B in Example 1.

FIG. 75 is a diagram in which a result of measuring a sub-threshold swing value SS is written on FIG. 72B in Example 1.

FIG. 76 is a block diagram illustrating an example of a schematic configuration of a vehicle control system.

FIG. 77 is a diagram illustrating examples of positions at which an outside-vehicle information detection unit and an imaging unit are installed.

FIG. 78 is a diagram illustrating an example of a schematic configuration of an endoscopic surgery system.

FIG. 79 is a block diagram illustrating an example of functional configurations of a camera head and a CCU.

### DESCRIPTION OF EMBODIMENTS

Hereinafter, the present disclosure will be described on the basis of examples with reference to the drawings. However, the present disclosure is not limited to the examples, and various numerical values and materials in the examples are examples. The description will be given in the following order.

1. Overall description regarding imaging element according to present disclosure, stacked imaging element according to present disclosure, solid-state imaging devices according to first and second aspects of present disclosure, and inorganic oxide semiconductor material according to present disclosure
2. Example 1 (imaging element according to present disclosure, stacked imaging element according to present disclosure, solid-state imaging device according to second aspect of present disclosure, and inorganic oxide semiconductor material according to present disclosure)
3. Example 2 (modification of Example 1)
4. Example 3 (modification of Examples 1 and 2; solid-state imaging device according to first aspect of present disclosure)
5. Example 4 (modification of Examples 1 to 3; imaging element including transfer control electrode)
6. Example 5 (modification of Examples 1 to 4; imaging element including charge discharging electrode)
7. Example 6 (modification of Examples 1 to 5; imaging element including a plurality of charge accumulation electrode segments)
8. Example 7 (modification of Examples 1 to 6; imaging element including charge movement control electrode)
9. Example 8 (modification of Example 7)
10. Example 9 (solid-state imaging device in first and second configurations)
11. Example 10 (modification of Example 9)

### 12. Others

<Overall Explanation Regarding Imaging Element According to Present Disclosure, Stacked Imaging Element According to Present Disclosure, Solid-State Imaging Devices According to First and Second Aspects of Present Disclosure, and Inorganic Oxide Semiconductor Material According to Present Disclosure>

In an imaging element according to the present disclosure, an imaging element according to the present disclosure configuring a stacked imaging element, and an imaging element according to the present disclosure configuring a solid-state imaging device according to first and second aspect of the present disclosure (hereinafter, these imaging elements may be collectively referred to as an “imaging element or the like according to the present disclosure”), it is possible to employ a configuration in which an optical gap of an inorganic oxide semiconductor material is equal to or greater than 2.7 eV and equal to or less than 3.2 eV and thereby not only to obtain the inorganic oxide semiconductor material layer as a layer that is transparent to incident light but also to eliminate a concern that some hindrance occurs in movement of charge from a photoelectric conversion layer to an inorganic oxide semiconductor material layer. In this case, when a composition of the inorganic oxide semiconductor material is represented as GaₐSn_{b}Zn_{c}O_{d} (where a+b+c=1.00, and a>0, b>0, and c>0), 0.45(b−0.62)≤0.55a≤0.45b . . . (1) is preferably satisfied.

Alternatively, in order to obtain the inorganic oxide semiconductor material layer as a layer that is transparent with respect to incident light in a wider wavelength range, the optical gap of the inorganic oxide semiconductor material is preferably equal to or greater than 3.0 eV and equal to or less than 3.2 eV, and in this case, 0.45(b−0.23)≤0.55a≤0.45b . . . (1′) is preferably satisfied.

The optical gap of the inorganic oxide semiconductor material depends mainly on proportions of gallium atoms and tin atoms (ratio between the numbers of atoms) in the composition of the inorganic oxide semiconductor material, and the optical gap value increases as the proportion of gallium atoms increases. In order for the inorganic oxide semiconductor material layer to be transparent in a visible light range, the optical gap is preferably equal to or greater than 2.7 eV as described above. On the other hand, in order to reliably receive charge generated in the photoelectric conversion layer by the inorganic oxide semiconductor material layer, the level of a conduction band of the inorganic oxide semiconductor material is required to be deeper than the level of a conduction band of a material configuring the photoelectric conversion layer, and to do so, the optical gap of the inorganic oxide semiconductor material is preferably equal to or less than 3.2 eV, for example, as described above.

As the proportion of tin atoms increases in the inorganic oxide semiconductor material, better conductivity can be obtained. Incidentally, how likely oxygen deficiency of the inorganic oxide semiconductor material occurs (in other words, the fact that an oxygen deficiency generation energy value is low) mainly depends on proportions of gallium atoms and tin atoms (the ratio between the numbers of atoms) in the composition of the inorganic oxide semiconductor material, and oxygen deficiency of the inorganic oxide semiconductor material is more likely to occur as the proportion of the tin atoms increases, and as a result, crystal defects are more likely to occur. Since the inorganic oxide semiconductor material layer is provided to accumulate the charge generated in the photoelectric conversion layer and transfer the charge to the first electrode, occurrence of carriers due to crystal defects and oxygen deficiency in the inorganic oxide semiconductor material layer may lead to an increase in carrier density and an increase in dark current and may deteriorate an S/N ratio of the imaging element. Therefore, presence of gallium atoms and zinc atoms can compensate for oxygen deficiency occurring.

Also, since the inorganic oxide semiconductor material layer is provided to transfer the charge generated in the photoelectric conversion layer to the first electrode, it takes time to read a signal from the imaging element if a transfer speed is low, and it is not possible to obtain an appropriate frame rate required by the solid-state imaging device. In order to increase the transfer speed, it is necessary to increase carrier mobility, that is, field mobility in the inorganic oxide semiconductor material layer. In regard to a relationship between the proportions of gallium atoms and zinc atoms (a ratio between the numbers of atoms) in the composition of the inorganic oxide semiconductor material and the carrier mobility, there is a trend that the carrier mobility value decreases as the proportions of the zinc atoms and the gallium atoms working as impurities relative to tin atoms which contribute to conductivity increase.

In the imaging element or the like according to the present disclosure including the aforementioned preferable configuration, it is possible to employ a configuration in which oxygen deficiency generation energy of the inorganic oxide semiconductor material is equal to or greater than 2.6 eV, and in this case, when the composition of the inorganic oxide semiconductor material is represented as GaₐSn_{b}Zn_{c}O_{d} (where a+b+c=1.00, and a>0, b>0, and c>0), a≤−3.0(b−0.63) . . . (2) is preferably satisfied.

Alternatively, it is possible to employ a configuration in which the oxygen deficiency generation energy of the inorganic oxide semiconductor material is equal to or greater than 2.8 eV, and in this case, when the composition of the inorganic oxide semiconductor material is represented as GaₐSn_{b}Zn_{c}O_{d} (where a+b+c=1.00, and a>0, b>0, and c>0), a≤−3.0(b−0.55) . . . (2-1) and a≤−11.0(b−0.50) . . . (2-2) are preferably satisfied.

Alternatively, it is possible to employ a configuration in which the oxygen deficiency generation energy of the inorganic oxide semiconductor material is equal to or greater than 3.0 eV, and in this case, when the composition of the inorganic oxide semiconductor material is represented as GaₐSn_{b}Zn_{c}O_{d} (where a+b+c=1.00, and a>0, b>0, and c>0), a≤−3.0(b−0.45) . . . (2-3) and 7.0(b−0.3)≤3.0a . . . (2-4) are preferably satisfied.

Here, the oxygen deficiency generation energy is energy required to generate oxygen deficiency, and it is possible to state that as the oxygen deficiency generation energy value increases, oxygen deficiency is less likely to occur (oxygen atoms are less likely to be desorbed), oxygen atoms and molecules and other atoms and molecules are less likely to be taken, and a stable state is achieved. The oxygen deficiency generation energy can be obtained by first principle calculation, for example. Note that since the inorganic oxide semiconductor material layer includes a plurality of kinds of metal atoms, “oxygen deficiency generation energy of the metal atoms” indicates an average value of oxygen deficiency generation energy that the plurality of kinds of metal atoms in the inorganic oxide semiconductor material have. Alternatively, since there is a case in which the carrier mobility value is low when the oxygen deficiency generation energy value is high, the oxygen deficiency generation energy of the inorganic oxide semiconductor material is preferably equal to or greater than 2.6 eV and equal to or less than 3.0 eV in such a case. Moreover, in the imaging element or the like according to the present disclosure including the aforementioned preferable configuration, it is possible to employ a configuration in which the carrier mobility of the inorganic oxide semiconductor material layer is equal to or greater than 10 cm²/V·s, and when the composition of the inorganic oxide semiconductor material is represented as GaₐSn_{b}Zn_{c}O_{d} (where a+b+c=1.00, and a>0, b>0, and c>0), b>0.23 . . . (3) is satisfied, and it is thus possible to quickly move the charge accumulated in the inorganic oxide semiconductor material layer to the first electrode.

The various preferable modes described above can be applied to the inorganic oxide semiconductor material according to the present disclosure.

Moreover, in the imaging element or the like according to the present disclosure including the aforementioned preferable modes and configurations, it is possible to employ a mode in which a value ΔEN (=ENₐₙᵢₒₙ−EN_{cation}) obtained by subtracting an average value EN_{cation} of electronegativity of cation species configuring the inorganic oxide semiconductor material layer from an average value ENₐₙᵢₒₙ of electronegativity of anion species configuring the inorganic oxide semiconductor material layer is less than 1.695. Here, when the inorganic oxide semiconductor material layer is represented as (A¹ₐ₁A²ₐ₂A³ₐ₃ . . . A^{M}_{aM})(B¹_{b1}B²_{b2}B³_{b3} . . . B^{N}_{bN}) [where A¹, A², A³, . . . , A^{M} are cation species, B¹, B², B³, . . . B^{N} are anion species, a1, a2, a3, . . . , aM, b1, b2, b3, . . . , bN are values corresponding to atomic percentages, and a total is 1.00], it is possible to employ a mode in which representations of ENₐₙᵢₒₙ=(B1×b1+B2×b2+B3×b3 . . . +BN×bN)/(b1+b2+b3 . . . +bN) and EN_{cation}=(A1×a1+A2×a2+A3×a3 . . . +AM×aM)/(a1+a2+a3 . . . +aM) are established.

Here, B1, B2, B3, . . . , BN are electronegativities of the anion species B¹, B², B³, . . . , B^{N}, and A1, A2, A3, . . . , AM are electronegativities of the cation species A¹, A², A³, . . . , AM. Specifically, the cation species include Ga, Sn, and Sn, while the anion species include 0. Here, in order for ΔEN (=ENₐₙᵢₒₙ−EN_{cation}) to be less than 1.695, 3.44−1.81×a−1.65×c−1.96×b≤1.695 . . . (4) is preferably satisfied.

Alternatively, in the imaging element or the like according to the present disclosure, when the composition of the inorganic oxide semiconductor material is represented as GaₐSn_{b}Zn_{c}O_{d} (where a+b+c=1.00, and a>0, b>0, and c>0), it is preferable that values of a, b, and c satisfy Expression (1) below, or satisfy Expression (2) below, or satisfy Expression (3) below, or satisfy expressions (1) and (2) below, or satisfy Expressions (1) and (3) below, or satisfy Expressions (2) and (3) below, or satisfy Expressions (1), (2), and (3) below, and further, it is more preferable that a, b, c values satisfy Expressions (1), (2), and (3) below. Here,

0.45(b−0.62)≤0.55a≤0.45b (1)

a≤−3.0(b−0.63) (2)

b≥0.23 (3)

The composition of the inorganic oxide semiconductor material layer can be obtained on the basis of ICP emission spectrometry (inductively coupled radiofrequency plasma emission spectrometry, ICP-AES) or X-ray photoelectron spectroscopy (XPS), for example. Other impurities such as hydrogen, other kinds of metal, or metal compounds may be mixed in a process of forming the inorganic oxide semiconductor material layer in some cases, and mixing in of a minute amount (equal to or less than 3% in terms of molar fraction, for example) thereof will not be a hindrance.

Moreover, in the imaging element or the like according to the present disclosure including the aforementioned preferable configuration, it is preferable to employ a mode in which the carrier concentration of the inorganic oxide semiconductor material layer is equal to or greater than 1×10¹⁴ cm⁻³ and equal to or less than 1×10¹⁷ cm⁻³, and it is thus possible to achieve an increase in amount of charge accumulated in the inorganic oxide semiconductor material layer. Furthermore, in the imaging element or the like according to the present disclosure including these preferable modes and various preferable configurations, which will be described later, it is preferable to employ a mode in which the carrier mobility of the inorganic oxide semiconductor material layer is equal to or greater than 10 cm²/V·s, and it is thus possible to quickly move the charge accumulated in the inorganic oxide semiconductor material layer to the first electrode. The various preferable modes described above can be applied to the inorganic oxide semiconductor material according to the present disclosure.

Furthermore, in the imaging element or the like according to the present disclosure including the preferable modes and configurations described above, it is possible to employ a mode in which the photoelectric conversion unit further includes an insulating layer and a charge accumulation electrode that is disposed to be separated from the first electrode and is disposed to face the inorganic oxide semiconductor material layer via the insulating layer.

In regard to the first electrode, the second electrode, the charge accumulation electrode, and the photoelectric conversion layer, detailed description will be given later.

Furthermore, in the imaging element or the like according to the present disclosure including the preferable modes and configurations described above, it is possible to employ a mode in which the charge generated in the photoelectric conversion layer moves to the first electrode via the inorganic oxide semiconductor material layer, and in this case, it is possible to employ a mode in which the charge is electrons.

Moreover, in the imaging element or the like according to the present disclosure including the preferable modes and configurations described above, it is preferable to employ a mode in which the inorganic oxide semiconductor material layer is configured of a first layer and a second layer from the side of the first electrode, and when an average film density in the first layer from a boundary between the first electrode and the inorganic oxide semiconductor material layer to 3 nm, preferably to 5 nm, or more preferably to 10 nm is defined as ρ₁ and an average film density in the second layer is defined as ρ₂, ρ₁>5.9 g/cm³ and ρ₁−ρ₂≥0.1 g/cm³, preferably ρ₁≥6.1 g/cm³ and ρ₁−ρ₂≥0.2 g/cm³ are satisfied.

Note that although a thinner thickness of the first layer is better, the minimum layer thickness is defined as 3 nm since it is necessary to prevent a discontinuous layer from being formed. Also, the maximum layer thickness is defined as 10 nm since an excessively thick thickness may cause degradation of properties of the inorganic oxide semiconductor material layer. It is possible to employ a mode in which the composition of the first layer and the composition of the second layer are the same. Alternatively, it is possible to employ a mode in which the inorganic oxide semiconductor material layer is configured of a first layer and a second layer, the composition of the first layer and the composition of the second layer are the same, and when an average film density in the first layer from a boundary between the first electrode and the inorganic oxide semiconductor material layer to 3 nm, preferably to 5 nm, or more preferably to 10 nm is defined as ρ₁ and an average film density in the second layer is defined as ρ₂, ρ₁−ρ₂≥0.1 g/cm³, preferably ρ₁−ρ₂>0.2 g/cm³ are satisfied.

Alternatively, it is possible to employ a mode in which when an average oxygen deficiency generation energy of the first layer from the boundary between the first electrode and the inorganic oxide semiconductor material layer to 3 nm, preferably to 5 nm, or more preferably to 10 nm is defined as E_{OD-1′}, and an average oxygen deficiency generation energy of the second layer is defined as E_{OD-2′}, E_{OD-1′}>2.8 eV and E_{OD-1′}−E_{OD-2′}>0.2 eV, preferably E_{OD-1′}>2.9 eV and E_{OD-1′}−E_{OD-2′}≥0.3 eV are satisfied.

Alternatively, it is possible to employ a mode in which the composition of the first layer and the composition of the second layer are the same and E_{OD-1′}−E_{OD-2′} >0.2 eV, preferably E_{OD-1′}−E_{OD-2′}>0.3 eV is satisfied.

Film density can be obtained on the basis of an X-ray reflectivity (XRR) method. Here, the XRR method is a method for determining a film thickness and film density of a sample by causing X rays to be incident on the surface of the sample at a very shallow angle, measuring an intensity profile of the X rays reflected in a mirror surface direction with respect to the incident angle, comparing the obtained intensity profile of the X rays with a simulation result, and optimizing simulation parameters.

The imaging element according to the present disclosure including such an inorganic oxide semiconductor material layer configured of the first layer and the second layer can be obtained by a method for manufacturing an imaging element including a photoelectric conversion unit configured of a first electrode, and a photoelectric conversion layer containing an organic material and a second electrode being laminated, the inorganic oxide semiconductor material layer configured of the first layer and the second layer being formed from the side of the first electrode between the first electrode and the photoelectric conversion layer, the method including a step of: forming the first layer by a sputtering method and then forming the second layer on the basis of the sputtering method with a smaller input power than an input power when the first layer is formed.

As a result of various tests, the fact that there is a relationship that an average density linearly increases as the an input power increases between the input power and the average film density when the inorganic oxide semiconductor material layer is formed on the basis of a sputtering method was discovered. Here, it is thought that an orientation of the inorganic oxide semiconductor material is aligned and the inorganic oxide semiconductor material layer becomes fine in a case in which the input power is high, while the orientation of the inorganic oxide semiconductor material is less likely to be aligned and the inorganic oxide semiconductor material layer becomes coarse in a case in which the input power is low.

By forming the inorganic oxide semiconductor material layer configured of the first layer and the second layer from the side of the first electrode between the first electrode and the photoelectric conversion layer and defining the thickness of the first layer and the relationship between the average film density ρ₁ in the first layer and the average film density ρ₂ in the second layer in this manner, it is possible to eliminate the concern that damage occurs in the underlayer when the first layer is formed and to obtain an imaging element with excellent properties.

Moreover, in the imaging element or the like according to the present disclosure including the preferable modes and configurations described above, it is possible to employ a mode in which a protective layer containing an inorganic oxide is formed between the photoelectric conversion layer and the inorganic oxide semiconductor material layer. The oxygen deficiency generation energy (the energy required to generate oxygen deficiency) of metal atoms configuring the protective layer is preferably equal to or greater than 5 eV (or in another expression, equal to or greater than 4.5 eV). In these cases, when the oxygen deficiency generation energy of the metal atoms configuring the protective layer is defined as E_{OD-1}, and the oxygen deficiency generation energy of the metal atoms configuring the inorganic oxide semiconductor material layer is defined as E_{OD-2}, E_{OD-1}−E_{OD-2}≥1 eV is preferably satisfied, and further, in these cases, the oxygen deficiency generation energy E_{OD-2} of the metal atoms configuring the inorganic oxide semiconductor material layer is preferably equal to or greater than 3 eV and yet preferably equal to or greater than 4 eV.

Note that in a case in which the protective layer contains a plurality of kinds of metal atoms, the “oxygen deficiency generation energy of the metal atoms” indicates an average value of the oxygen deficiency generation energies that the plurality of kinds of metal atoms have.

Moreover, in the imaging element or the like according to the present disclosure including the preferable modes and configurations described above, it is possible to employ a mode in which the protective layer prevents invasion of hydrogen into the inorganic oxide semiconductor material layer. By preventing invasion of hydrogen into the inorganic oxide semiconductor material layer, oxygen atoms pulled out of the inorganic oxide semiconductor material layer due to the invasion of hydrogen are curbed, and it is thus possible to curb occurrence of oxygen deficiency and to obtain an inorganic oxide semiconductor material layer with stable properties. Note that hydrogen that may invade the inorganic oxide semiconductor material layer is present in the photoelectric conversion layer or is present due to a process of manufacturing the imaging element. In regard to a hydrogen blocking ability of the protective layer measured by a thermal desorption spectroscopy method (TDS method), a hydrogen ion relative strength ratio detected at the time of heating at 350° C. measured by the TDS method is preferably equal to or less than 0.1 when the strength ratio at the time of heating titanium is assumed to be 1.0. In the thermal desorption spectroscopy method, a sample is heated in a vacuum, a partial pressure of desorbed hydrogen is measured, and it is thus possible to obtain a relationship between a hydrogen desorption speed and a sample temperature. Specifically, the sample is placed on a stage, and the sample is heated by irradiating the sample with infrared rays from a portion below the stage. Temperature control is performed by a thermocouple provided on the side of the stage. It is also possible to measure the temperature on the side of the sample surface by a thermocouple on the side of the upper portion of the sample. Gas generated by the heating is positively ionized through collision against accelerated electrons and is separated in accordance with a mass charge ratio. It is thus possible to detect hydrogen ions.

Moreover, in the imaging element or the like according to the present disclosure including the preferable modes and configurations described above, when an absolute value of energy (a symbol of the value is negative) is defined as becoming larger as a level from a vacuum level becomes higher on the assumption that the vacuum level is a zero reference, and when an energy average value of maximum energy values in the conduction band of the inorganic oxide semiconductor material layer is defined as E₂, and an energy average value of LUMO values in the photoelectric conversion layer is defined as E₁, E₁−E₂≥0.1 (eV) is preferably satisfied, and E₁−E₂>0.1 (eV) is more preferably satisfied. Note that the “minimum energy” means that the absolute value of the energy value is a minimum while “maximum energy” means that the absolute value of the energy value is a maximum. The same applies to the following description. By defining the relationship between E₁ and E₂ in this manner, an energy barrier between the photoelectric conversion layer and the inorganic oxide semiconductor material layer is reduced, and it is possible to achieve reliable charge movement from the photoelectric conversion layer to the inorganic oxide semiconductor material layer and to curb hole dropping.

The energy average value E₂ of the maximum energy values in the conduction band of the inorganic oxide semiconductor material layer is defined to be an average value in the inorganic oxide semiconductor material layer. Also, the energy average value E₁ of the LUMO values of the photoelectric conversion layer is defined to be an average value in a portion of the photoelectric conversion layer located in the vicinity of the inorganic oxide semiconductor material layer. Here, the “portion of the photoelectric conversion layer located in the vicinity of the inorganic oxide semiconductor material layer” indicates a portion of the photoelectric conversion layer located in a region within 10% of the thickness of the photoelectric conversion layer (that is, the region of 0% to 10% of the thickness of the photoelectric conversion layer) with reference to the boundary between the inorganic oxide semiconductor material layer and the photoelectric conversion layer.

Energy in a valence band and a HOMO value can be obtained on the basis of, for example, an ultraviolet photoelectron spectroscopy method (UPS method). Also, the energy in a conduction band and a LUMO value can be obtained from {(energy in the valence band, a HOMO value)+E_{b}}. Furthermore, a band gap energy E_{b} can be obtained on the basis of the following expression from a wavelength λ (an optical absorption end wavelength; the unit is nm) of optical absorption.

E_{b}=hv=h(c/λ)=1239.8/λ [eV]

Moreover, in the imaging element or the like according to the present disclosure including the various preferable modes and configurations described above, it is possible to employ a mode in which the inorganic oxide semiconductor material layer is amorphous (for example, amorphia which locally does not have a crystalline structure). Whether or not the inorganic oxide semiconductor material layer is amorphous can be determined on the basis of an X-ray diffraction analysis. However, the inorganic oxide semiconductor material layer is not limited to being amorphous, and may have a crystalline structure, or may have a polycrystalline structure.

Moreover, in the imaging element or the like according to the present disclosure including the various preferable modes and configurations described above, it is desirable that the thickness of the inorganic oxide semiconductor material layer be 1×10⁻⁸ m to 1.5×10⁻⁷ m, preferably 2×10⁻⁸ m to 1.0×10⁻⁷ m, more preferably 3×10⁻⁸ m to 1.0×10⁻⁷ m.

Furthermore, in the imaging element or the like according to the present disclosure including the various preferable modes and configurations described above, the light is incident from the second electrode, a surface roughness Ra of the surface of the inorganic oxide semiconductor material layer at the boundary between the photoelectric conversion layer and the inorganic oxide semiconductor material layer (in a case in which a protective layer is formed, the boundary between the protective layer and the inorganic oxide semiconductor material layer) is equal to or less than 1.5 nm, and a value of a root mean square roughness Rq of the surface of the inorganic oxide semiconductor material layer is preferably equal to or less than 2.5 nm.

The surface roughness Ra and Rq is based on the definitions of JIS B0601:2013. Smoothness of the surface of the inorganic oxide semiconductor material layer at the boundary between such a photoelectric conversion layer and the inorganic oxide semiconductor material layer (in a case in which a protective layer is formed, a boundary between the protective layer and the inorganic oxide semiconductor material layer) can curb scattering reflection at the surface of the inorganic oxide semiconductor material layer and can lead to an improvement in bright current properties in photoelectric conversion. The surface roughness Ra of the surface of the charge accumulation electrode is preferably equal to or less than 1.5 nm, and the root mean square roughness Rq of the surface of the charge accumulation electrode is preferably equal to or less than 2.5 nm.

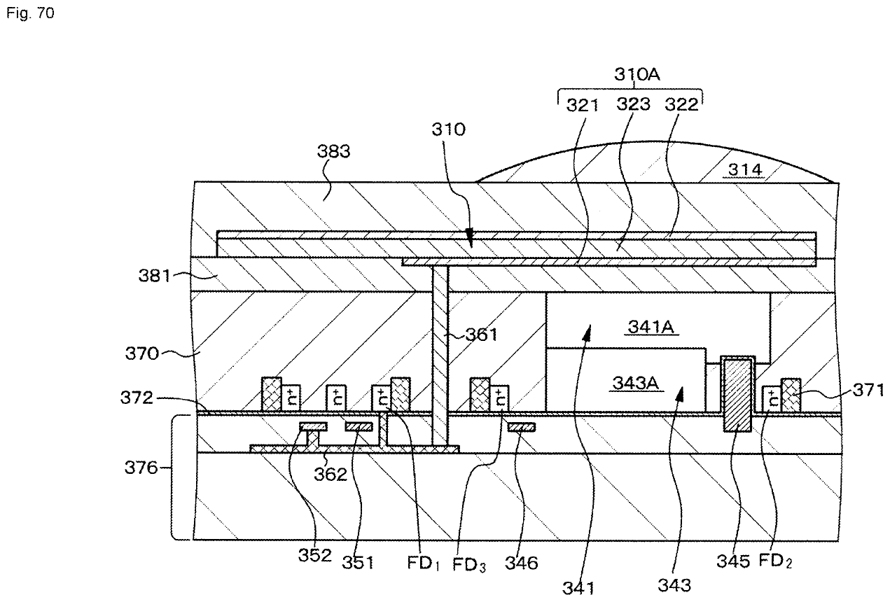

In the imaging element in the related art illustrated in FIG. 70, charge generated through photoelectric conversion in the second photoelectric conversion unit 341A and the third photoelectric conversion unit 343A is temporarily accumulated in the second photoelectric conversion unit 341A and the third photoelectric conversion unit 343A and is then transferred to the second floating diffusion layer FD₂ and the third floating diffusion layer FD₃. Therefore, it is possible to achieve full depletion of the second photoelectric conversion unit 341A and the third photoelectric conversion unit 343A. However, charge generated through photoelectric conversion in the first photoelectric conversion unit 310A is accumulated directly in the first floating diffusion layer FD₁. Therefore, it is difficult to achieve full depletion of the first photoelectric conversion unit 310A. As a result of the above, there is a concern that kTC noise may increase, random noise may deteriorate, and this may lead to a degradation of quality of captured images.

As long as the imaging element or the like according to the present disclosure includes the charge accumulation electrode that is disposed to be separated from the first electrode and is disposed to face the inorganic oxide semiconductor material layer via the insulating layer as described above, it is possible to accumulate charge in the inorganic oxide semiconductor material layer (in some cases, the inorganic oxide semiconductor material layer and the photoelectric conversion layer, or the inorganic oxide semiconductor material layer, the protective layer, and the photoelectric conversion layer) when the photoelectric conversion unit is irradiated with light and photoelectric conversion is performed in the photoelectric conversion unit. It is thus possible to achieve full depletion of the charge accumulation unit and cancel out charge when the exposure is started. As a result, it is possible to curb occurrence of the phenomenon that kTC noise increases, random noise deteriorates, and this leads to degradation of quality of captured images. Note that in the following description, the inorganic oxide semiconductor material layer, or the inorganic oxide semiconductor material layer and the photoelectric conversion layer, or the inorganic oxide semiconductor material layer, the protective layer, and the photoelectric conversion layer may be collectively referred to as “the inorganic oxide semiconductor material layer and the like”.

The inorganic oxide semiconductor material layer may have a single-layer configuration or a multilayer configuration. Also, the inorganic oxide semiconductor material located above the charge accumulation electrode and the inorganic oxide semiconductor material located above the first electrode may be different. The protective layer may also have a single-layer configuration or a multilayer configuration.

The inorganic oxide semiconductor material layer and the protective layer can be formed on the basis of a physical vapor deposition method (PVD method), specifically, a sputtering method. More specifically, it is possible to use, as a sputtering device, a parallel plate sputtering device, a DC magnetron sputtering device, or an RF sputtering device, for example, and it is possible to exemplify a sputtering method using argon (Ar) gas as process gas and using a desired sintered body as a target. It is only necessary to use a GaₐSn_{b}Zn_{c}O_{d} sintered body as a target. However, the method for forming the inorganic oxide semiconductor material layer is not limited to a PVD method such as a sputtering method or a deposition method, and it is also possible to form the inorganic oxide semiconductor material layer on the basis of a coating method or the like. Also, it is possible to exemplify an atomic layer deposition method (ALD method) as a method for forming the protective layer.

Note that in a case in which the inorganic oxide semiconductor material layer is formed on the basis of a sputtering method, it is possible to control the energy level of the inorganic oxide semiconductor material layer by controlling the amount of introduced oxygen gas (oxygen gas partial pressure). Specifically, it is possible to perform the control on the basis of an oxygen gas partial pressure=(O₂ gas pressure)/(total pressure of Ar gas and O₂ gas) when it is formed on the basis of the sputtering method.

The oxygen gas partial pressure is preferably 0.005 to 0.10. Moreover, in the imaging element or the like according to the present disclosure, it is possible to employ a mode in which the content of oxygen in the inorganic oxide semiconductor material layer is lower than the content of oxygen in the stoichiometric composition. Here, it is possible to control the energy level of the inorganic oxide semiconductor material layer on the basis of the content of oxygen and to deepen the energy level as the content of oxygen becomes increasingly lower than the content of oxygen in the stoichiometric composition, that is, as oxygen deficiency increases.

As the imaging element or the like according to the present disclosure, it is possible to exemplify a CCD element, a CMOS image sensor, and a signal amplification-type image sensor of a contact image sensor (CIS) and a charge modulation device (CMD). Solid-state imaging devices according to the first and second aspects of the present disclosure and solid-state imaging devices with first and second configurations, which will be described later, can configure, for example, a digital still camera, a video camera, a camcorder, a monitor camera, an in-vehicle camera, a smartphone camera, a game user interface camera, and a biometrics camera.

### Example 1

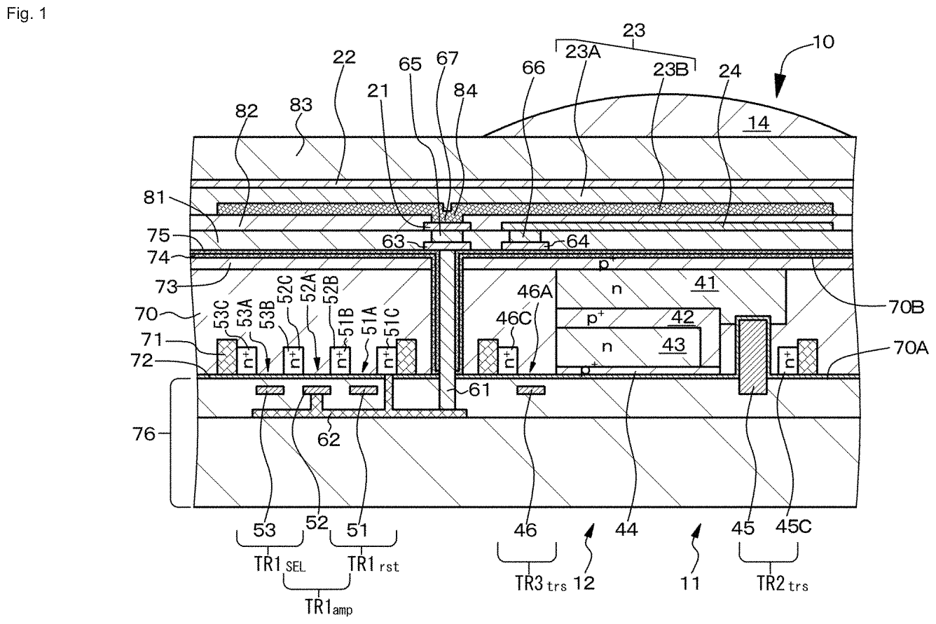

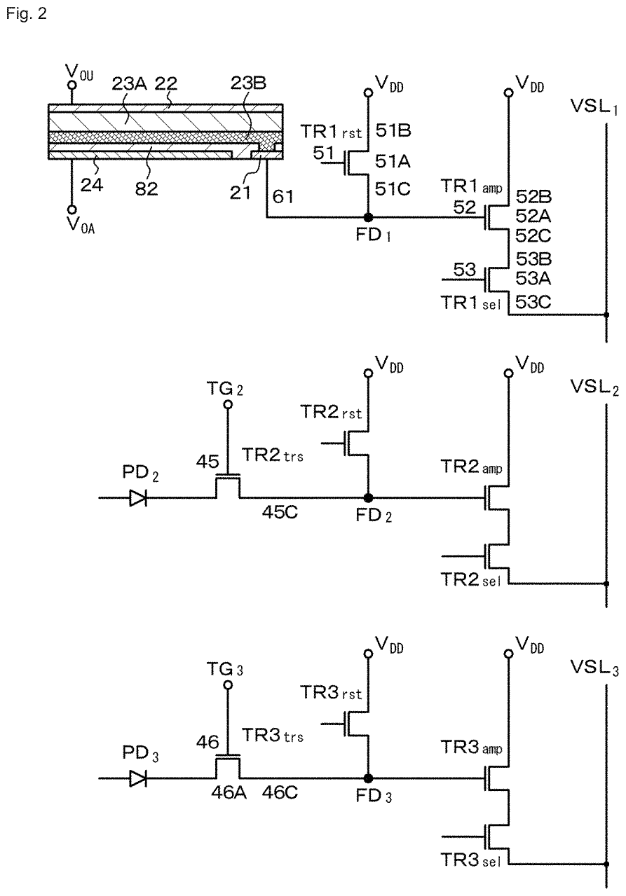

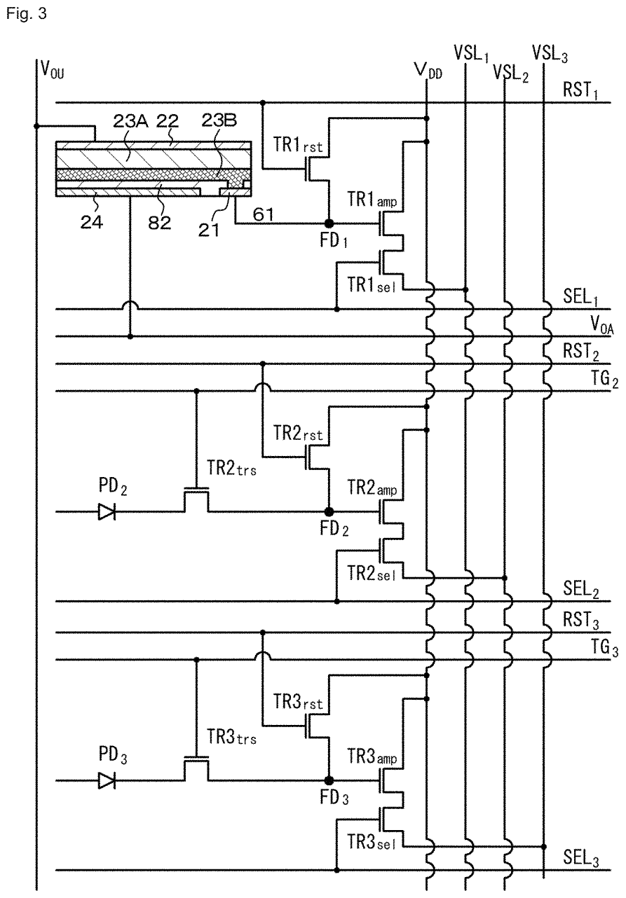

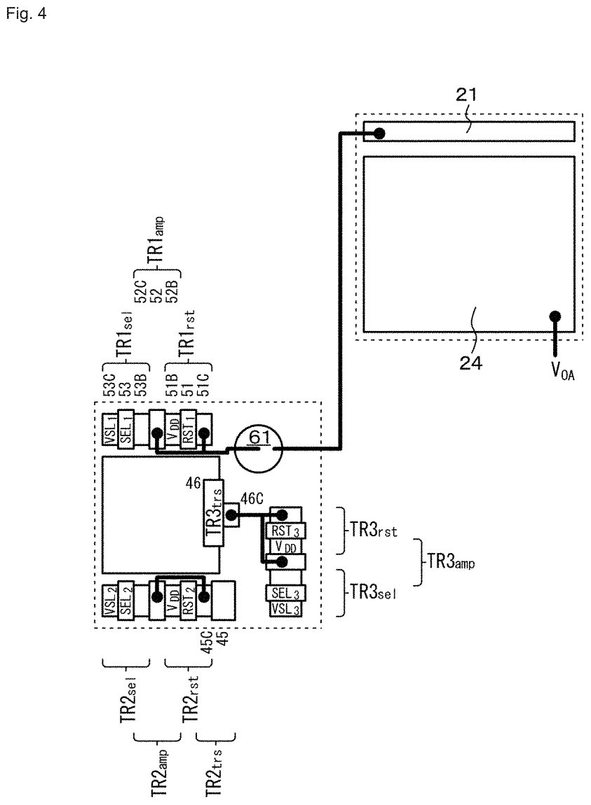

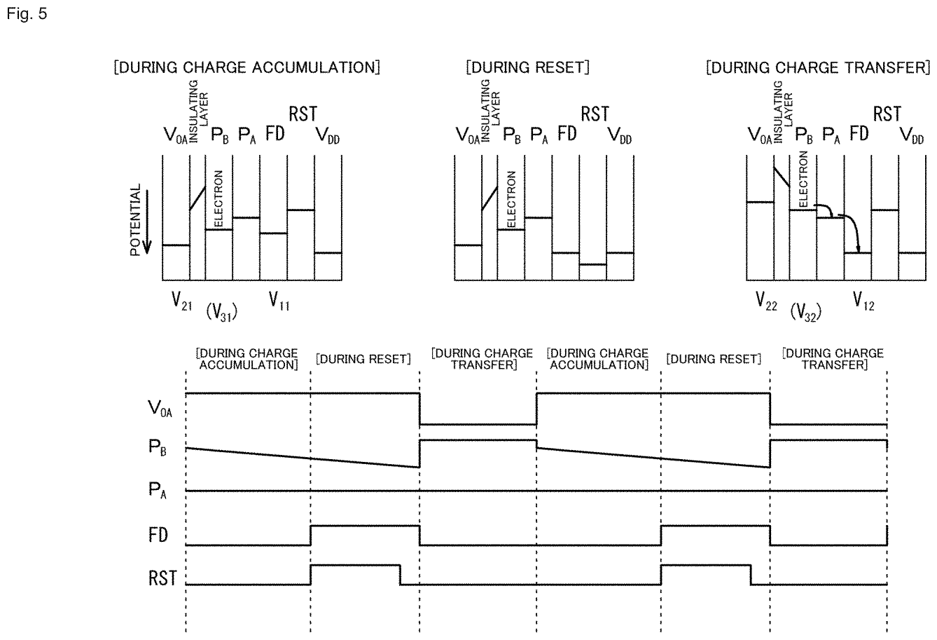

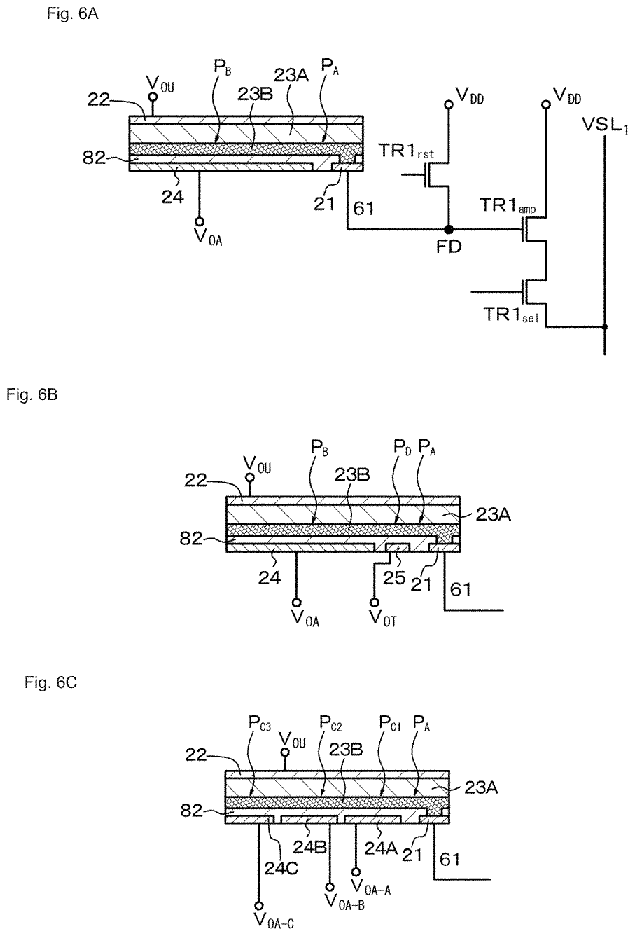

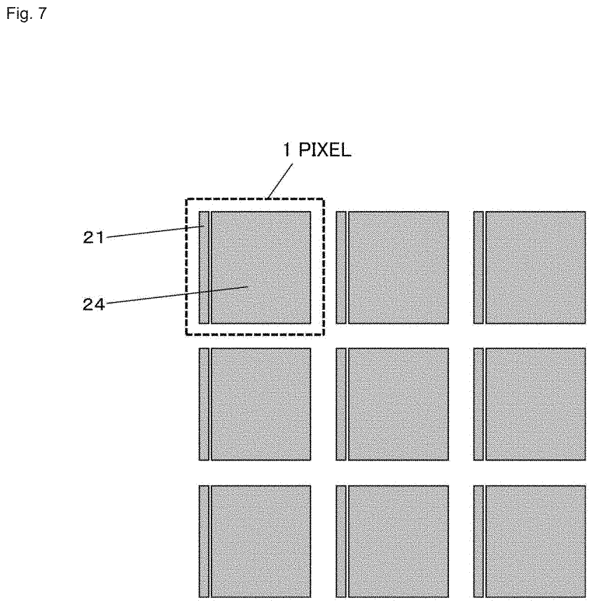

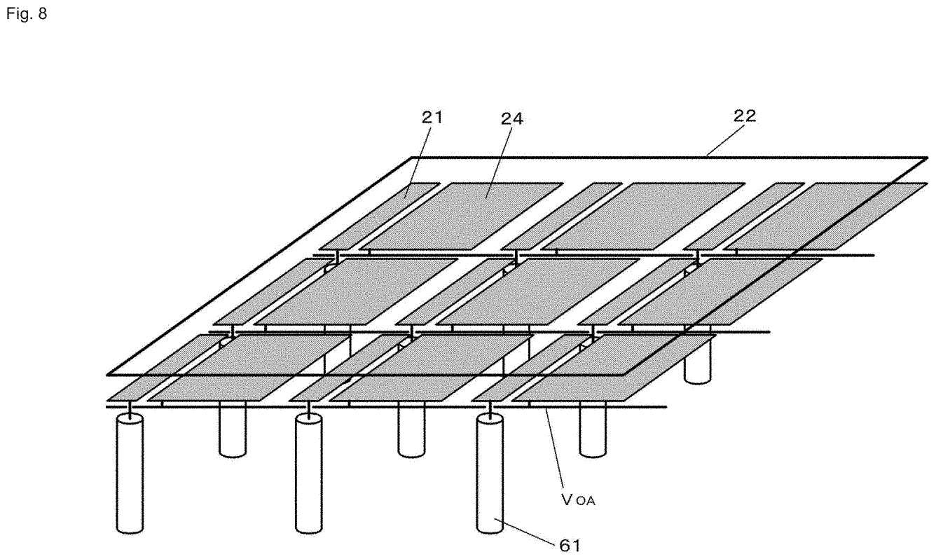

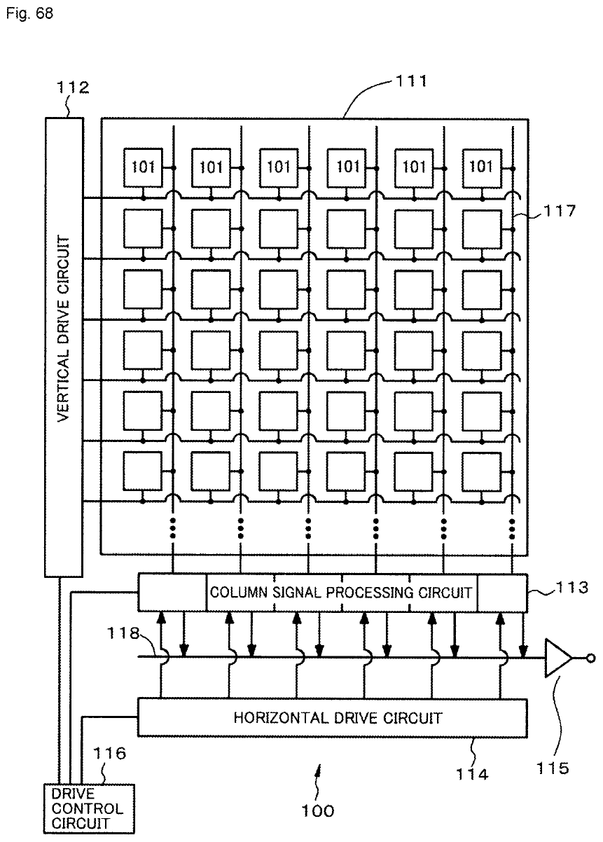

Example 1 relates to an imaging element according to the present disclosure, a stacked imaging element according to the present disclosure, a solid-state imaging device according to a second aspect of the present disclosure, and an inorganic oxide semiconductor material according to the present disclosure. A schematic partial sectional view of an imaging element and a stacked imaging element (hereinafter, simply referred to as an “imaging element”) in Example 1 is illustrated in FIG. 1, an equivalent circuit diagram of the imaging element in Example 1 is illustrated in FIGS. 2 and 3, a schematic disposition diagram of a first electrode and a charge accumulation electrode configuring a photoelectric conversion unit of the imaging element in Example 1 and transistors configuring a control unit is illustrated in FIG. 4, a state of a potential at each portion when the imaging element in Example 1 is operating is schematically illustrated in FIG. 5, and an equivalent circuit diagram for explaining each portion of the imaging element in Example 1 is illustrated in FIG. 6A. Also, a schematic disposition diagram of the first electrode and the charge accumulation electrode configuring the photoelectric conversion unit of the imaging element in Example 1 is illustrated in FIG. 7, and a schematic transparent perspective view of the first electrode, the charge accumulation electrode, a second electrode, and a contact hole portion is illustrated in FIG. 8. Moreover, a conceptual diagram of the solid-state imaging device in Example 1 is illustrated in FIG. 68.

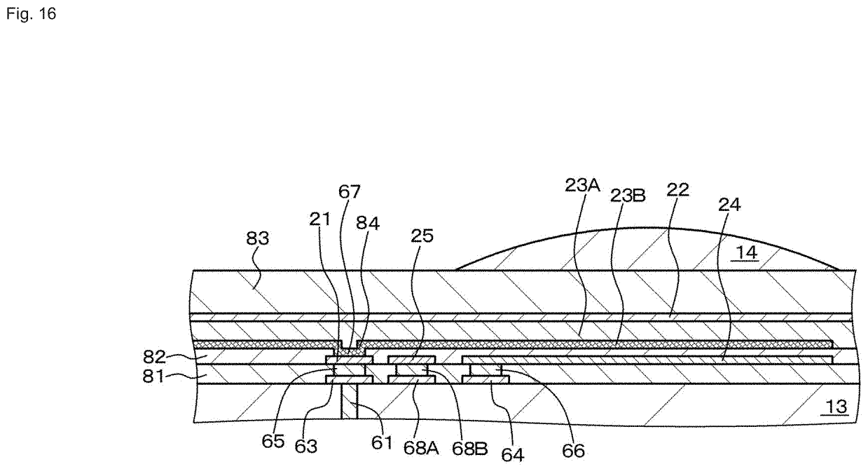

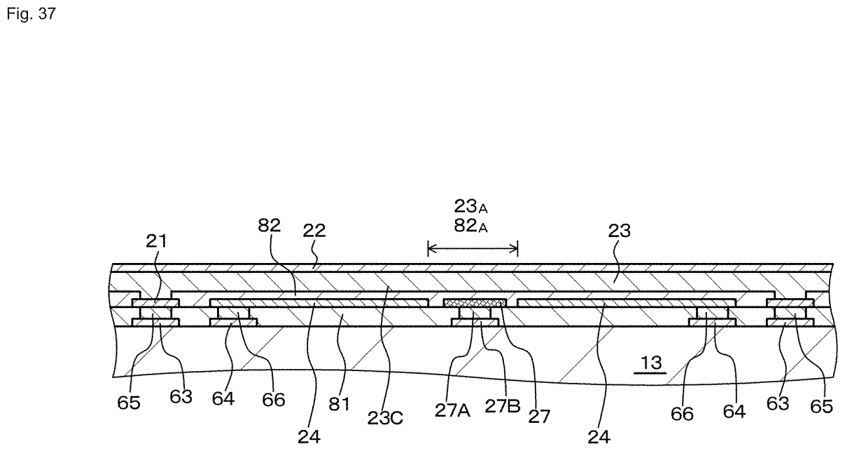

Note that in FIGS. 37, 43, 46A, 46B, 47A, and 47B, illustration of the photoelectric conversion layer 23A and the inorganic oxide semiconductor material layer 23B is omitted and the photoelectric conversion layer 23A and the inorganic oxide semiconductor material layer 23B are collectively illustrated as a photoelectric conversion laminated body 23. In FIGS. 16, 25, 28, 37, 43, 46A, 46B, 47A, 47B, 66, and 67, various imaging element components located below an interlayer insulating layer 81 are collectively illustrated with the reference sign 13 in order to simplify the drawings for convenience.

The imaging element in Example 1 includes a photoelectric conversion unit that is configured of a first electrode 21, and a photoelectric conversion layer 23A containing an organic material and a second electrode 22 being laminated, and an inorganic oxide semiconductor material layer 23B is formed between the first electrode 21 and the photoelectric conversion layer 23A. Also, an inorganic oxide semiconductor material configuring the inorganic oxide semiconductor material layer 23B contains gallium (Ga) atoms, tin (Sn) atoms, zinc (Zn) atoms, and oxygen (O) atoms. The inorganic oxide semiconductor material layer 23B does not contain any indium (In) atoms. In other words, the inorganic oxide semiconductor material layer 23B is configured of a composite oxide containing gallium (Ga) atoms, tin (Sn) atoms, and zinc (Zn) atoms, and specifically, the inorganic oxide semiconductor material layer 23B is configured of a composite oxide including a gallium oxide, a tin oxide, and a zinc oxide.

The stacked imaging element in Example 1 includes at least one imaging element in Example 1. Also, the solid-state imaging device in Example 1 includes a plurality of stacked imaging elements in Example 1. Additionally, the solid-state imaging device in Example 1 configures, for example, a digital still camera, a video camera, a camcorder, a monitor camera, a vehicle-mounted camera (in-vehicle camera), a smartphone camera, a game user interface camera, a biometrics camera, and the like.

Also, the composition of the inorganic oxide semiconductor material in Example 1 is represented as GaₐSn_{b}Zn_{c}O_{d} (where a+b+c=1.00, and a>0, b>0, and c>0), and it is desirable that a, b, and c values satisfy Expression (1) below, or satisfy Expression (1′) below, or satisfy Expression (2) below, or satisfy Expressions (2-1) and (2-2) [or Expressions (2-3) and (2-4)] below, or satisfy Expression (3) below, or satisfy Expressions (1) and (2) below, or satisfy Expressions (1′) and (2) below, or satisfy Expressions (1), (2-1), and (2-2) [or Expressions (2-3) and (2-4)] below, or satisfy Expressions (1′) and (2-1), and (2-2) [or Expressions (2-3) and (2-4)] below, or satisfy Expressions (1) and (3) below, or satisfy Expressions (1′) and (3) below, or satisfy Expressions (2) and (3) below, or satisfy Expressions (2-1) and (2-2) [or Expressions (2-3) and (2-4)] and (3) below, or satisfy Expressions (1), (2), and (3), below, or satisfy Expressions (1′), (2), and (3) below, or satisfy Expressions (1), (2-1) and (2-2) [or Expressions (2-3) and (2-4)] and (3) below, or satisfy Expressions (1′), (2-1) and (2-2) [or Expressions (2-3) and (2-4)] and (3) below, and further preferably satisfy Expressions (1), (2), and (3) below, and yet further preferably satisfy Expressions (1), (2-1), (2-2), and (3) below.

Note that d=1.5a+2.0b+c is preferably satisfied.

Here, 0.45(b−0.62)≤0.55a≤0.45b (1)

0.45(b−0.23)≤0.55a≤0.45b (1′)

a≤−3.0(b−0.63) (2)

a≤−3.0(b−0.55) (2-1)

a≤−11.0(b−0.50) (2-2)

a≤−3.0(b−0.45) (2-3)

7.0(b−0.3)≤3.0a (2-4)

b>0.23 (3)

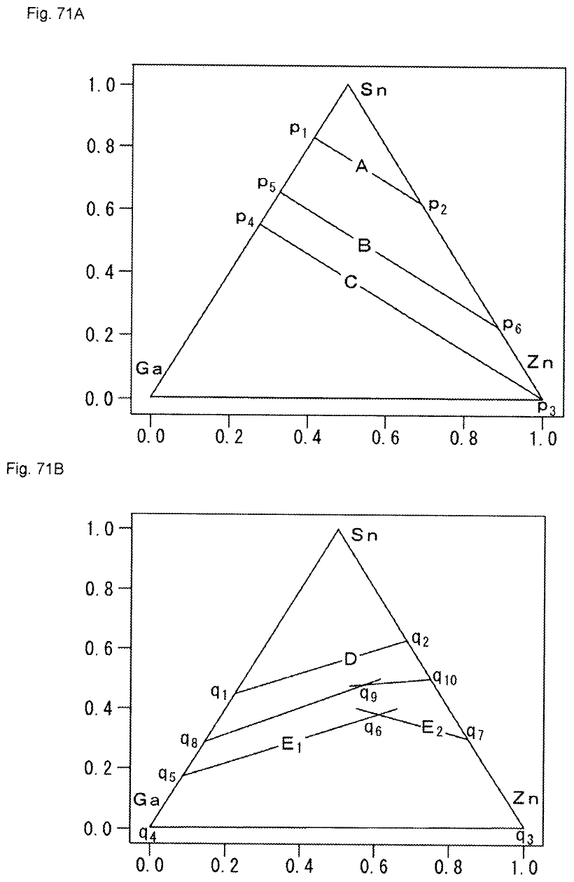

In the imaging element in Example 1, the optical gap of the inorganic oxide semiconductor material is preferably equal to or greater than 2.7 eV and equal to or less than 3.2 eV. Here, as illustrated in the graph in FIG. 71A, it is possible to achieve the optical gap of the inorganic oxide semiconductor material of equal to or greater than 2.7 eV and equal to or less than 3.2 eV by satisfying 0.45(b−0.62)≤0.55a≤0.45b . . . (1).

Note that in FIG. 71A, the solid line “A” represents (a, b): 0.45(b−0.62)=0.55a with which an optical gap of 2.7 eV can be obtained.

Also, the solid line “B” represents (a, b): 0.45(b−0.23)=0.55a with which an optical gap of 3.0 eV can be obtained.

Furthermore, the solid line “C” represents (a, b):0.55a=0.45b with which an optical gap of 3.2 eV can be obtained.

A region that satisfies Expression (1) is a region obtained by connecting the point p₁, the point p₂, the point p₃, the point p₄, and the point p₁. Additionally, a region that satisfies 0.45(b−0.23)≤0.55a≤0.45b . . . (1′) is a region obtained by connecting the point p₅, the point p₆, the point p₃, the point p₄, and the point p₅.

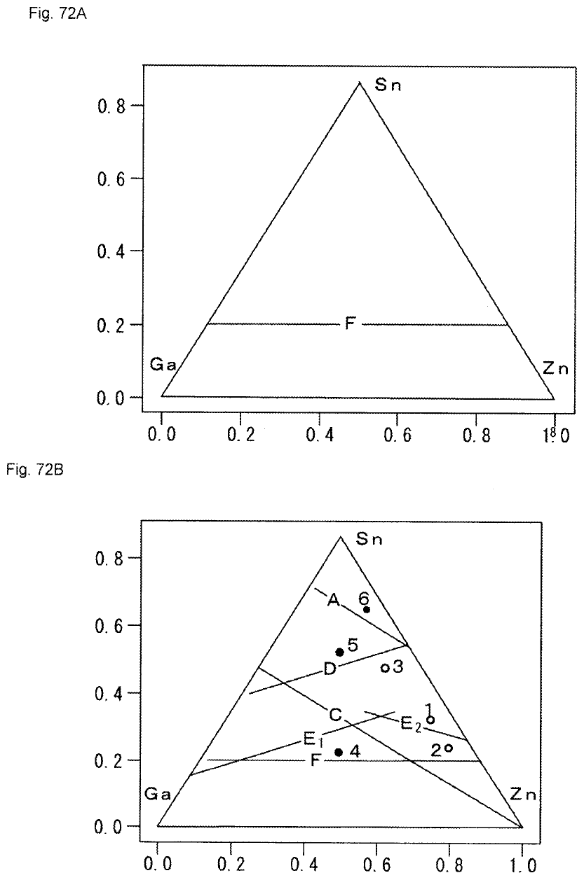

In FIGS. 71A, 71B, and 72A, values that correlate with values of an optical gap, carrier mobility, and oxygen deficiency generation energy were obtained by performing simulation on GaₐSn_{b}Zn_{c}O_{d} in which values (a, b, c) of the composition were changed in various manners to obtain electron state density and performing first principle calculation. Also, the values (a, b, c) with which desired values of the optical gap, the oxygen deficiency generation energy, and the carrier mobility were obtained were plotted with straight lines on the basis of the obtained values.

Also, in the imaging element in Example 1, oxygen deficiency generation energy of the inorganic oxide semiconductor material is preferably equal to or greater than 2.6 eV. In this case, it is possible to achieve the oxygen deficiency generation energy of the inorganic oxide semiconductor material of equal to or greater than 2.6 eV by satisfying a≤−3.0(b−0.63) . . . (2).

Alternatively, in the imaging element in Example 1, the oxygen deficiency generation energy of the inorganic oxide semiconductor material is preferably equal to or greater than 2.8 eV. Here, it is possible to achieve the oxygen deficiency generation energy of the inorganic oxide semiconductor material of equal to or greater than 2.8 eV by satisfying a≤−3.0(b−0.55) . . . (2-1) and a≤−11.0(b−0.50) . . . (2-2).

Alternatively, in the imaging element in Example 1, the oxygen deficiency generation energy of the inorganic oxide semiconductor material is preferably equal to or greater than 3.0 eV. Here, it is possible to achieve the oxygen deficiency generation energy of the inorganic oxide semiconductor material of equal to or greater than 3.0 eV by satisfying a≤−3.0(b−0.45) . . . (2-3) and 7.0(b−0.3)≤3.0a . . . (2-4).

Alternatively, in the imaging element in Example 1, the oxygen deficiency generation energy of the inorganic oxide semiconductor material is preferably equal to or greater than 2.6 eV and equal to or less than 3.0 eV. Here, it is possible to achieve an oxygen deficiency generation energy of the inorganic oxide semiconductor material of equal to or greater than 2.6 eV and equal to or less than 3.0 eV by satisfying a≤−3.0(b−0.63) . . . (2), a>−3.0(b−0.45) . . . (2′-3) and 7.0(b−0.3)>3.0a . . . (2′-4).

Note that in FIG. 71B, the solid line “D” illustrates (a, b): a=−3.0(b−0.63) with which it is possible to obtain the oxygen deficiency generation energy of 2.6 eV. Also, the solid lines “E₁, E₂” illustrate (a, b): a=−3.0 (b−0.45) and 7.0(b−0.3)=3.0a with which it is possible to obtain the oxygen deficiency generation energy of 3.0 eV.

Moreover, the solid lines “E₃, E₄” illustrate (a, b): a=−3.0 (b−0.55) and a=−11.0(b−0.50) with which it is possible to obtain the oxygen deficiency generation energy of 2.8 eV.

A region that satisfies Expression (2) is a region obtained by connecting the point q₁, the point q₂, the point q₃, the point q₄, and the point q₁. Also, a region that satisfies Expressions (2-1) and (2-2) is a region obtained by connecting the point q₈, the point q₉, the point q₁₀, the point q₃, the point q₄, and the point q₈. A region that satisfies Expressions (2-3) and (2-4) is a region obtained by connecting the point q₅, the point q₆, the point q₇, the point q₃, the point q₄, and the point q₅.

Alternatively, in the imaging element in Example 1, carrier mobility of the inorganic oxide semiconductor material layer 23B is equal to or greater than 10 cm²/V·s. Here, it is possible to achieve the carrier mobility of the inorganic oxide semiconductor material layer of equal to or greater than 10 cm²/V·s by satisfying b>0.23 . . . (3) as illustrated in FIG. 72A.

Note that in FIG. 72A, the solid line “F” illustrates (a, b): b=0.23 with which it is possible to obtain the carrier mobility of 10 cm²/V·s.

As illustrated in FIG. 72B and Table 1 below, imaging elements including inorganic oxide semiconductor material layers with changed a, b, c values were produced as trial, and optical gaps, occurrence of oxygen deficiency, and carrier mobility were evaluated. Here, the mark “A” in “oxygen deficiency” in Table 1 indicates that oxygen deficiency is very unlikely to occur, the mark “B” indicates that oxygen deficiency is unlikely to occur, the mark “C” indicates that oxygen deficiency is likely to occur, and the mark “D” indicates that oxygen deficiency is very likely to occur. Also, the mark “A” in “mobility” in Table 1 indicates that carrier mobility is very high, and the mark “B” indicates that carrier mobility is high. In FIG. 72B, the white circle 1 indicates Example 1A, the white circle 2 indicates Example 1B, the white circle 3 indicates Example 1C, the black circle 4 indicates Comparative Example 1A, the black circle 5 indicates Comparative Example 1B, and the black circle 6 indicates Comparative Example 1C. It is possible to ascertain that the imaging elements in Examples 1A, 1B, and 1C that satisfy Expressions (1), (2), and (3) have excellent properties in terms of optical gaps, occurrence of oxygen deficiency, and carrier mobility. In Comparative Example 1A, the imaging element is excellent in terms of the carrier mobility and the occurrence of oxygen deficiency while the optical gap value is 3.3 eV. In Comparative Example 1B, the imaging element is excellent in terms of the carrier mobility and the optical gap while oxygen deficiency is likely to occur. Furthermore, in Comparative Example 1C, the imaging element is excellent in terms of the carrier mobility while oxygen deficiency is very likely to occur and the optical gap value is 2.6 eV.

TABLE 1

| | | | | Optical | Oxygen |
|---|---|---|---|---|---|
| Mobility | a | b | c | gap | deficiency |
| Example 1A | 0.0625 | 0.5625 | 0.3750 | 2.9 eV | B |
| B | | | | | |
| Example 1B | 0.0625 | 0.6625 | 0.2750 | 3.0 eV | A |
| B | | | | | |
| Example 1C | 0.10 | 0.35 | 0.55 | 2.9 eV | B |
| A | | | | | |
| Comarative | 0.37 | 0.37 | 0.26 | 3.3 eV | B |
| Example 1A | | | | | |
| B | | | | | |
| Comparative | 0.20 | 0.20 | 0.60 | 2.9 eV | C |
| Example 1B | | | | | |
| A | | | | | |
| Comparative | 0.05 | 0.20 | 0.75 | 2.6 eV | D |
| Example 1C | | | | | |
| A | | | | | |

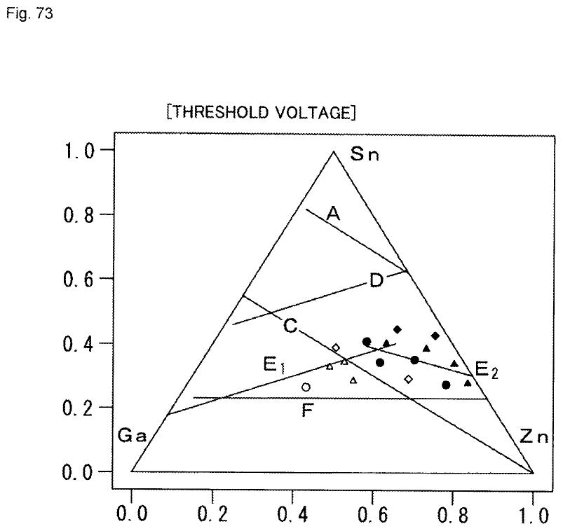

Thin film transistors (TFTs) with channel formation regions configured of inorganic oxide semiconductor material layers were produced as trial. Composition ratio dependency of threshold voltages Vₜₕ of the TFTs corresponds to oxygen deficiency generation energy. In other words, the oxygen deficiency generation energy increases, the oxygen deficiency is less likely to be generated, and as a result, the threshold voltages Vₜₕ increase as the composition ratio of Ga or Zn (that is, a value of a ratio a of the composition of Ga or a value of a ratio c of the composition of Zn) increases. Here, the threshold voltages Vₜₕ are defined as gate voltages when a current starts to flow through the channel formation regions in the TFTs. A drawing in which results of measuring the threshold voltages Vₜₕ are written in FIG. 72B is illustrated in FIG. 73, and the symbols in FIG. 73 indicate the following ranges of the values of the threshold voltages Vₜₕ (unit: volt).

Value of threshold voltage Vₜₕ
White circle . . . 18 to 21
White triangle . . . 15 to 18
White diamond . . . 12 to 15
Black circle . . . 9 to 12
Black triangle . . . 6 to 9
Black diamond . . . 3 to 6

Negative values of the threshold voltages Vₜₕ mean that electrons that can be carriers caused by oxygen deficiency are present before a channel portion is induced by a positive gate voltage. Positive values of the threshold voltages Vₜₕ correspond to the fact that no electrons that can be carriers at a gate voltage of 0 V are present and electrons induced by an application of a positive gate voltage are thus initially used to fill a trap potential and do not contribute to induction of the channel portion. Note that in a case in which the values of the threshold voltages Vₜₕ are positive, it is possible to intentionally lower the values of the threshold voltages Vₜₕ by reducing oxygen partial pressures when the inorganic oxide semiconductor material layers are formed.

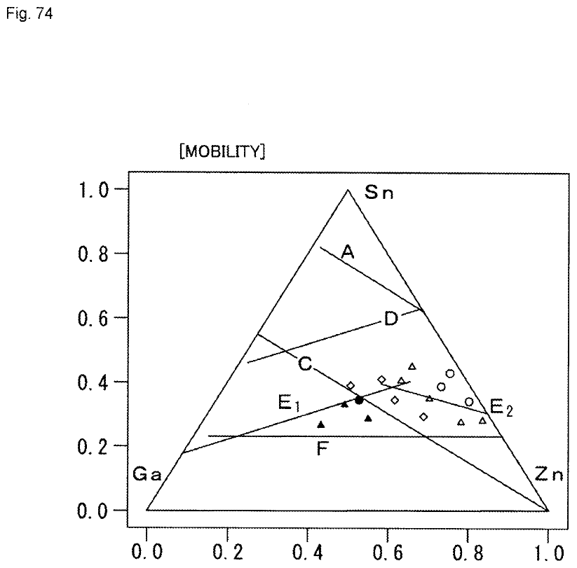

Carrier mobility μ has a positive correlation with the concentration of Sn. This is considered to be because spreading of 4s trajectory that contributes to conduction of electrons further curbed by Ga than by Zn. Also, it is possible to ascertain that Zn lowers the value of the carrier mobility μ to a lower extent than expected. The drawing in which results of measuring the carrier mobility μ are written in FIG. 72B is illustrated in FIG. 74, and the symbols in FIG. 74 represents the following ranges of values of the carrier mobility μ (unit: cm²/V·s)

Value of carrier mobility μ
White circle . . . 12 to 15
White triangle . . . 9 to 12
White diamond . . . 6 to 9
Black circle . . . 3 to 6
Black triangle . . . 0 to 3

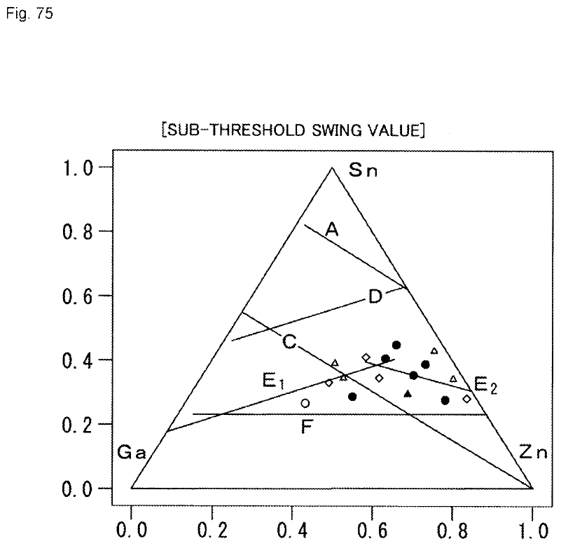

A diagram in which results of measuring sub-threshold swing values SS are written in FIG. 72B is illustrated in FIG. 75, and the symbols in FIG. 75 illustrate the following ranges of the values of the sub-threshold swing values SS (unit: V/dec).

Sub-threshold swing values SS
White circle . . . 0.8 to 1.0
White triangle . . . 0.6 to 0.8
White diamond . . . 0.4 to 0.6
Black circle . . . 0.2 to 0.4
Black triangle . . . 0.0 to 0.2
Above data is illustrated.

It was possible to ascertain from various tests that the values of the threshold voltages Vₜₕ of the TFTs falling the range of equal to or less than 15 volts were preferable for the imaging elements. Also, it was possible to ascertain that the values of the carrier mobility μ of the TFTs falling the range of equal to or greater than 6 cm²/V·s is preferable for the imaging elements. Additionally, it was possible to ascertain from the aforementioned results of measuring the threshold voltages Vₜₕ and carrier mobility μ that the a, b, and c values preferably satisfied all Expressions (1), (2), and (3) above as described above. Also, it was possible to confirm from the measurement of the sub-threshold swing values SS that the TFTs reliably operated.

The carrier concentration of the inorganic oxide semiconductor material layer 23B is equal to or greater than 1×10¹⁴ cm⁻³ and equal to or less than 1×10¹⁷ cm⁻³, and as a result, it is possible to increase the amount of charge accumulated in the inorganic oxide semiconductor material layer 23B. Note that in a case in which GaₐSn_{b}Zn_{c}O_{d} configuring the inorganic oxide semiconductor material layer is used as a channel structure portion of the thin film transistor (TFT), carrier density of equal to or greater than about 10¹⁹/cm³ is needed unlike the case in which it is used for an imaging element. Also, the carrier mobility of the inorganic oxide semiconductor material layer 23B is equal to or greater than 10 cm²/V·s, the inorganic oxide semiconductor material layer 23B is amorphous, and the thickness of the inorganic oxide semiconductor material layer 23B is 1×10⁻⁸ m to 1.5×10⁻⁷ m.

Here, in Example 1, the photoelectric conversion unit further includes an insulating layer 82 and a charge accumulation electrode 24 that is disposed to be separated from the first electrode 21 and is disposed to face the inorganic oxide semiconductor material layer 23B via the insulating layer 82. The inorganic oxide semiconductor material layer 23B is in contact with a region, which is in contact with the first electrode 21, and the insulating layer 82, is in contact with a region, in which the charge accumulation electrode 24 is not present, and the insulating layer 82 on the lower side, and has, on the lower side, a region in which the charge accumulation electrode 24 is present. Also, light is incident from the second electrode 22, surface roughness Ra of the surface of the inorganic oxide semiconductor material layer 23B at the boundary between the photoelectric conversion layer 23A and the inorganic oxide semiconductor material layer 23B is equal to or less than 1.5 nm, and a value of root mean square roughness Rq of the surface of the inorganic oxide semiconductor material layer 23B is equal to or less than 2.5 nm. Charge generated in the photoelectric conversion layer 23A moves to the first electrode 21 via the inorganic oxide semiconductor material layer 23B. In this case, the charge is electrons.

Also, when an energy average value of maximum energy values in a conduction band of the inorganic oxide semiconductor material layer 23B is defined as E₂, and an energy average value of LUMO values of the photoelectric conversion layer 23A is defined as E₁, the following Expression, namely E₁−E₂>0.1 (eV) is satisfied, or preferably, the following expression is satisfied.

E₁−E₂>0.1 (eV)

It is possible to control the energy level of the inorganic oxide semiconductor material layer 23B by controlling the amount of introduced oxygen gas (oxygen gas partial pressure) when the inorganic oxide semiconductor material layer 23B is formed on the basis of the sputtering method. The oxygen gas partial pressure is preferably set to 0.005 (0.5%) to 0.10 (10%).

Results of obtaining a relationship between the oxygen gas partial pressure and the energy level obtained by an inverse photoemission spectroscopy when the film thickness of the inorganic oxide semiconductor material layer 23B is 50 nm, and the inorganic oxide semiconductor material layer 23B is configured of GaₐSn_{b}Zn_{c}O_{d} are illustrated in Table 2 below, and it is possible to control the energy level of the inorganic oxide semiconductor material layer 23B by controlling the amount of introduced oxygen gas (oxygen gas partial pressure) when the inorganic oxide semiconductor material layer 23B is formed on the basis of the sputtering method for the imaging element in Example 1.

a+b+c+d=1.00

a/(a+b+c)=0.25

b/(a+b+c)=0.45

c/(a+b+c)=0.30

TABLE 2

| Oxygen gas | |
|---|---|
| partial pressure | Energy level |
| 0.5% | 4.63 eV |
| 10.0% | 4.74 eV |

Next, in regard to the photoelectric conversion layer 23A and the inorganic oxide semiconductor material layer 23B, an energy level of the inorganic oxide semiconductor material layer 23B, an energy level difference (E₂−E₁) between the photoelectric conversion layer 23A and the inorganic oxide semiconductor material layer 23B and mobility of a material configuring the inorganic oxide semiconductor material layer 23B were examined. As illustrated in Table 3, three different conditions were applied. In other words, under a first condition, IGZO was used as the material configuring the inorganic oxide semiconductor material layer 23B, and under second and third conditions, GaₐSn_{b}Zn_{c}O_{d} illustrated below was used as the material configuring the inorganic oxide semiconductor material layer 23B. Also, the film thickness of the inorganic oxide semiconductor material layer 23B was set to 50 nm. Further, the photoelectric conversion layer 23A was made of quinacridone, and the thickness thereof was set to 0.1 μm. The LUMO value E₁ of a portion of the material configuring the photoelectric conversion layer 23A located in the vicinity of the inorganic oxide semiconductor material layer 23B was set to 4.5 eV. Note that when the inorganic oxide semiconductor material layer 23B was formed on the basis of the sputtering method, it is possible to obtain the imaging elements or the like based on the second condition and the third condition using targets with different compositions.

### Second Condition

a+b+c+d=1.00

a/(a+b+c)=0.27

b/(a+b+c)=0.42

c/(a+b+c)=0.31

### Third Condition

a+b+c+d=1.00

a/(a+b+c)=0.31

b/(a+b+c)=0.36

c/(a+b+c)=0.33

Under the first condition, the energy level difference (E₂−E₁) was 0 eV. Under the second condition, the energy level difference (E₂−E₁) was improved as compared with the first condition. As illustrated in Table 3, under the third condition, mobility was further improved as compared with the second condition.

TABLE 3

| | First | Second | Third |
|---|---|---|---|
| | condition | condition | condition |
| Inorganic oxide | 4.5 eV | 4.63 eV | 4.74 eV |
| semiconductor | | | |
| material layer | | | |
| Energy level difference | 0.0 eV | 0.13 eV | 0.24 eV |
| (E₂ − E₁) | | | |
| Mobility (unit: cm²/V · s) | 9 | 13 | 18 |

Transfer properties under these three conditions were evaluated in device simulation on the basis of the imaging element with the structure illustrated in FIG. 1. Note that a LUMO value E₁ of the photoelectric conversion layer 23A was set to 4.5 eV. The relative amount of electrons in a state in which electrons were attracted above the charge accumulation electrode 24 was set to 1×10⁰. Also, the relative amount of electrons in a state in which all the electrons attracted above the charge accumulation electrode 24 were transferred to the first electrode 21 was set to 1×10⁻⁴. Also, a time until all the electrons attracted above the charge accumulation electrode 24 were transferred to the first electrode 21 (referred to as a “transfer time”) was used as an indicator for determining how good the transfer properties were. Results of obtaining the transfer time are as illustrated in Table 4. The transfer time was further shortened under the second condition than under the first condition and was further shortened under the third condition than under the second condition. In other words, more excellent transfer property results were achieved as the value of (E₂−E₁) increased, and this indicates the result that such formation to obtain a larger LUMO value E₂ of the inorganic oxide semiconductor material layer 23B than a LUMO value E₁ of the photoelectric conversion layer 23A is a further preferable factor to further improve the transfer properties.

TABLE 4

| | Transfer time |
|---|---|
| First condition | 5.2 × 10⁻⁶ seconds |
| Second condition | 1.5 × 10⁻⁷ seconds |
| Third condition | 2.8 × 10⁻⁸ seconds |

It was possible to ascertain from a result of X-ray diffraction of the inorganic oxide semiconductor material layer 23B, the inorganic oxide semiconductor material layer 23B is amorphous (for example, amorphia that locally does not have a crystalline structure). Moreover, surface roughness Ra of the inorganic oxide semiconductor material layer 23B at the boundary between the photoelectric conversion layer 23A and the inorganic oxide semiconductor material layer 23B is equal to or less than 1.5 nm, and a value of root mean square roughness Rq of the inorganic oxide semiconductor material layer is equal to or less than 2.5 nm. Specifically, values before annealing were Ra=0.5 nm and Rq=2.5 nm, and values after annealing were Ra=0.5 nm and Rq=1.4 nm. Also, the surface roughness Ra of the charge accumulation electrode 24 was equal to or less than 1.5 nm, and the value of the root mean square roughness Rq of the charge accumulation electrode 24 was equal to or less than 2.5 nm. Specifically, Ra=0.5 nm and Rq=2.4 nm.

Light permeability of the inorganic oxide semiconductor material layer 23B with respect to light with a wavelength of 400 nm to 660 nm was equal to or greater than 65% (specifically, 82%), and light permeability of the charge accumulation electrode 24 with respect to light with a wavelength of 400 nm to 660 nm was also equal to or greater than 65% (specifically, 73%). A sheet resistance value of the charge accumulation electrode 24 was 3×10Ω/□ to 1×10³Ω/□ (specifically, 78Ω/□).

In Example 1, the inorganic oxide semiconductor material layer contains gallium (Ga) atoms, tin (Sn) atoms, and zinc (Zn) atoms. Therefore, it is possible to achieve, with a satisfactory balance, a decrease in carrier concentration (carrier density and a degree of depletion of the inorganic oxide semiconductor material layer) of the inorganic oxide semiconductor material layer, an improvement in carrier mobility, optimization of an optical gap, control of the energy average value E₂ of maximum energy value in the conduction band of the inorganic oxide semiconductor material layer, and curbing of occurrence of oxygen deficiency in the inorganic oxide semiconductor material layer. As a result, it is possible to provide an imaging element, a stacked imaging element, and a solid-state imaging device with excellent transfer properties of charge accumulated in the photoelectric conversion layer regardless of their simple configurations and structures, and it is possible to provide an inorganic oxide semiconductor material that is suitable for use in these. In other words, it is possible to control the carrier concentration (a degree of depletion of the inorganic oxide semiconductor material layer) of the inorganic oxide semiconductor material layer by controlling the proportion of the gallium atoms among the atoms configuring the inorganic oxide semiconductor material layer, it is possible to control carrier mobility of the inorganic oxide semiconductor material layer and to control conductivity by controlling the proportions of the gallium atoms and the zinc atoms, it is possible to apply high electric conductivity to the inorganic oxide semiconductor material layer by controlling the proportion of the tin atoms, and further, it is estimated that control of an amorphous state of the inorganic oxide semiconductor material layer, control of surface smoothness, and control of the energy value E₂ can be achieved. In addition, it is possible to prevent recombination during charge accumulation due to the two-layer structure of the inorganic oxide semiconductor material layer and the photoelectric conversion layer, it is possible to further increase charge transfer efficiency of the charge accumulated in the photoelectric conversion layer to the first electrode, and to curb generation of a dark current. Moreover, it is possible to temporarily hold the charge generated in the photoelectric conversion layer and control a transfer timing or the like.

Hereinafter, overall explanation of the imaging element according to the present disclosure, the stacked imaging element according to the present disclosure, and a solid-state imaging device according to a second aspect of the present disclosure will be provided, and detailed description of the imaging element and the solid-state imaging device in Example 1 will then be provided. Signs representing potentials applied to various electrodes in the following description are listed in Table 5 below.

TABLE 5

| | Charge | Charge |
|---|---|---|
| | accumulation | transfer |
| | period | period |
| First electrode | V₁₁ | V₁₂ |
| Second electrode | V₂₁ | V₂₂ |
| Charge accumulation electrode | V₃₁ | V₃₂ |
| Charge movement control electrode | V₄₁ | V₄₂ |
| Transfer control electrode | V₅₁ | V₅₂ |
| Charge discharging electrode | V₆₁ | V₆₂ |

In the imaging element or the like according to the present disclosure including the preferable modes described above, the imaging element including the charge accumulation electrode may be referred to as an “imaging element or the like including the charge accumulation electrode according to the present disclosure” below for convenience.

In the imaging element or the like including the charge accumulation electrode according to the present disclosure, light permeability of the inorganic oxide semiconductor material layer with respect to light with a wavelength of 400 nm to 660 nm is preferably equal to or greater than 65%. Also, light permeability of the charge accumulation electrode with respect to light with a wavelength of 400 nm to 660 nm is also preferably equal to or greater than 65%. A sheet resistance value of the charge accumulation electrode is preferably 3×10 Ω/□ to 1×10³ Ω/□.

In the imaging element or the like including the charge accumulation electrode according to the present disclosure, it is possible to employ a mode in which a semiconductor substrate is further included and the photoelectric conversion unit is disposed above the semiconductor substrate. Note that the first electrode, the charge accumulation electrode, the second electrode, and various electrodes are connected to a drive circuit, which will be described later.

The second electrode located on the light incident side may be shared by a plurality of imaging elements. In other words, the second electrode can be a so-called solid electrode except for an imaging element or the like including an upper charge movement control electrode according to the present disclosure, which will be described later. The photoelectric conversion layer may be shared by a plurality of imaging elements, that is, one photoelectric conversion layer may be formed in the plurality of imaging elements, or the photoelectric conversion layer may be provided in each imaging element. Although the inorganic oxide semiconductor material layer or the like is preferably provided in each imaging element, the inorganic oxide semiconductor material layer or the like may be shared by a plurality of imaging elements in some cases. In other words, one inorganic oxide semiconductor material layer or the like may be formed in a plurality of imaging elements by providing a charge movement control electrode, which will be described later, between an imaging element and an imaging element, for example. In a case in which one common inorganic oxide semiconductor material layer or the like is formed in a plurality of imaging elements, it is desirable that end portions of the inorganic oxide semiconductor material layer or the like be covered at least with the photoelectric conversion layer in terms of protection of the end portions of the inorganic oxide semiconductor material layer or the like.

Moreover, in the imaging element or the like including the charge accumulation electrode according to the present disclosure including the various preferable modes described above, it is possible to employ a mode in which the first electrode extends in an opening portion provided in the insulating layer and is connected to the inorganic oxide semiconductor material layer. Alternatively, it is possible to employ a mode in which the inorganic oxide semiconductor material layer or the like extends in an opening portion provided in the insulating layer and the inorganic oxide semiconductor material layer is connected to the first electrode, and in this case, it is possible to employ a mode in which an edge portion of a top surface of the first electrode is covered with the insulating layer, the first electrode is exposed from a bottom surface of the opening portion, and when a surface of the insulating layer that is in contact with the top surface of the first electrode is defined as a first surface, and a surface of the insulating layer that is in contact with a portion of the inorganic oxide semiconductor material layer facing the charge accumulation electrode is defined as a second surface, a side surface of the opening portion has an inclination spreading from the first surface toward the second surface, and further, it is possible to employ a mode in which the side surface of the opening portion that has the inclination spreading from the first surface toward the second surface is located on the side of the charge accumulation electrode.

Moreover, in the imaging element or the like including the charge accumulation electrode according to the present disclosure including the various preferable modes described above, it is possible to employ a configuration in which a control unit that is provided in the semiconductor substrate and has a drive circuit is further included, the first electrode and the charge accumulation electrode are connected to the drive circuit, a potential V₁₁ is applied to a first electrode and a potential V₃₁ is applied to the charge accumulation electrode from the drive circuit, and charge is accumulated in the inorganic oxide semiconductor material layer or the like in a charge accumulation period, and a potential V₁₂ is applied to the first electrode and potential V₃₂ is applied to the charge accumulation electrode from the drive circuit, and charge accumulated in the inorganic oxide semiconductor material layer or the like is read by the control unit via the first electrode in the charge transfer period. Here, the potential of the first electrode is higher than the potential of the second electrode, and V₃₁>V₁₁ and V₃₂<V₁₂ are satisfied.

Moreover, in the imaging element or the like including the charge accumulation electrode according to the present disclosure including the various preferable modes described above, it is possible to employ a mode in which a charge movement control electrode is formed in a region that faces a region of the photoelectric conversion layer located between adjacent imaging elements via the insulating layer. Note that such a mode may be referred to as an “imaging element or the like including a lower charge movement control electrode according to the present disclosure” for convenience. Alternatively, it is possible to employ a mode in which the charge movement control electrode is formed above the region of the photoelectric conversion layer located between the adjacent imaging elements instead of the formation of the second electrode. Note that such a mode may be referred to as an “imaging element or the like including an upper charge movement control electrode according to the present disclosure” for convenience.

In the following description, the “region of the photoelectric conversion layer located between the adjacent imaging elements” will be referred to as a “region −A of the photoelectric conversion layer” for convenience, and the “region of the insulating layer located between the adjacent imaging elements” will be referred to as a “region −A of the insulating layer” for convenience. The region −A of the photoelectric conversion layer corresponds to the region −A of the insulating layer. Moreover, the “region between the adjacent imaging elements” will be referred to as a “region −a” for convenience.

In the imaging element or the like including the lower charge movement control electrode (a lower side charge movement control electrode and a charge movement control electrode located on the side opposite to the light incident side with reference to the photoelectric conversion layer) according to the present disclosure, the lower charge movement control electrode is formed in a region facing the region −A of the photoelectric conversion layer via the insulating layer. In other words, the lower charge movement control electrode is formed below a portion of the insulating layer (the region −A of the insulating layer) in the region (region −a) sandwiched between a charge accumulation electrode and a charge accumulation electrode, each of which configures each of the adjacent imaging elements. The lower charge movement control electrode is provided to be separated from the charge accumulation electrode. In other words, the lower charge movement control electrode is provided such that it surrounds the charge accumulation electrode and is separated from the charge accumulation electrode, and the lower charge movement control electrode is disposed to face the region −A of the photoelectric conversion layer via the insulating layer.

In the imaging element or the like including the lower charge movement control electrode according to the present disclosure, it is possible to employ a mode in which a control unit that is provided in the semiconductor substrate and has a drive circuit is further included, the first electrode, the second electrode, the charge accumulation electrode, and the lower charge movement control electrode are connected to the drive circuit, a potential V₁₁ is applied to the first electrode and a potential V₃₁ is applied to the charge accumulation electrode and a potential V₄₁ is applied to the lower charge movement control electrode from the drive circuit, and charge is accumulated in the inorganic oxide semiconductor material layer or the like in the charge accumulation period, and a potential V₁₂ is applied to the first electrode and a potential V₃₂ is applied to the charge accumulation electrode and a potential V₄₂ is applied to the lower charge movement control electrode from the drive circuit, and the charge accumulated in the inorganic oxide semiconductor material layer or the like is read by the control unit via the first electrode in the charge transfer period.

Here, V₃₁>V₁₁, V₃₁>V₄₁, and V₁₂>V₃₂>V₄₂ are satisfied.

The lower charge movement control electrode may be formed in the same level as the first electrode or the charge accumulation electrode or may be formed in a different level.

In the imaging element or the like including the upper charge movement control electrode (the upper-side charge movement control electrode and the charge movement control electrode located on the light incident side with reference to the photoelectric conversion layer) according to the present disclosure, the upper charge movement control electrode is formed above the region of the photoelectric conversion layer located between the adjacent imaging elements instead of formation of the second electrode, and the upper charge movement control electrode is provided to be separated from the second electrode. In other words,

[A] it is possible to employ a mode in which the second electrode is provided in each imaging element and the upper charge movement control electrode is provided above the region −A of the photoelectric conversion layer such that it surrounds at least a part of the second electrode and is separated from the second electrode, or
[B] it is possible to exemplify a mode in which the second electrode is provided in each imaging element, the upper charge movement control electrode is provided such that it surrounds at least a part of the second electrode and is separated from the second electrode, and a part of the charge accumulation electrode is present below the upper charge movement control electrode, or
[C] it is possible to exemplify a mode in which the second electrode is provided in each imaging element, the upper charge movement control electrode is provided such that it surrounds at least a part of the second electrode and is separated from the second electrode, a part of the charge accumulation electrode is present below the upper charge movement control electrode, and further, the lower charge movement control electrode is formed below the upper charge movement control electrode.

A potential generated through coupling between the upper charge movement control electrode and the second electrode may be applied to the region of the photoelectric conversion layer located below the region between the upper charge movement control electrode and the second electrode.

Also, in the imaging element or the like including the upper charge movement control electrode according to the present disclosure, it is possible to employ a mode in which a control unit that is provided in the semiconductor substrate and has a drive circuit is further included, the first electrode, the second electrode, the charge accumulation electrode, and the upper charge movement control electrode are connected to the drive circuit, a potential V₂₁ is applied to the second electrode and a potential V₄₁ is applied to the upper charge movement control electrode from the drive circuit, charge is accumulated in the inorganic oxide semiconductor material layer or the like in the charge accumulation period, and a potential V₂₂ is applied to the second electrode and a potential V₄₂ is applied to the upper charge movement control electrode from the drive circuit, and the charge accumulated in the inorganic oxide semiconductor material layer or the like is read by the control unit via the first electrode in the charge transfer period.

Here, V₂₁>V₄₁ and V₂₂>V₄₂ are satisfied.

The upper charge movement control electrode is formed in the same level as the second electrode.

Moreover, in the imaging element or the like including the charge accumulation electrode according to the present disclosure including various preferable modes described above, it is possible to employ a mode in which a transfer control electrode (charge transfer electrode) that is disposed to be separated from the first electrode and the charge accumulation electrode and is disposed to face the inorganic oxide semiconductor material layer via the insulating layer is further included between the first electrode and the charge accumulation electrode. The imaging element or the like including the charge accumulation electrode according to the present disclosure in such a mode will be referred to as an “imaging element or the like including the transfer control electrode according to the present disclosure” for convenience.

Also, in the imaging element or the like including the transfer control electrode according to the present disclosure, it is possible to employ a configuration in which a control unit that is provided in a semiconductor substrate and has a drive circuit is further included, the first electrode, the charge accumulation electrode, and the transfer control electrode are connected to the drive circuit, a potential V₁₁ is applied to the first electrode and a potential V₃₁ is applied to the charge accumulation electrode and a potential V₅₁ is applied to the transfer control electrode from the drive circuit, and charge is accumulated in the inorganic oxide semiconductor material layer or the like in the charge accumulation period, and a potential V₁₂ is applied to the first electrode and a potential V₃₂ is applied to the charge accumulation electrode and a potential V₅₂ is applied to the transfer control electrode from the drive circuit, and the charge accumulated in the inorganic oxide semiconductor material layer or the like is read by the control unit via the first electrode in the charge transfer period. Here, the potential of the first electrode is higher than the potential of the second electrode, and V₃₁>V₅₁ and V₃₂≤V₅₂≤V₁₂ are satisfied.

Moreover, in the imaging element or the like including the charge accumulation electrode according to the present disclosure including the various preferable modes described above, it is possible to employ a mode in which a charge discharging electrode that is disposed to be connected to the inorganic oxide semiconductor material layer and to be separated from the first electrode and the charge accumulation electrode is further included. The imaging element or the like including the charge accumulation electrode according to the present disclosure in such a mode will be referred to as an “imaging element or the like including the charge discharging electrode according to the present disclosure” for convenience. Also, in the imaging element or the like including the charge discharging electrode according to the present disclosure, it is possible to employ a mode in which the charge discharging electrode is disposed such that it surrounds the first electrode and the charge accumulation electrode (that is, in a frame shape). The charge discharging electrode can be shared by (commonly provided for) a plurality of imaging elements. In this case, it is possible to employ a mode in which the inorganic oxide semiconductor material layer or the like extends in a second opening portion provided in the insulating layer and is connected to the charge discharging electrode, an edge portion of a top surface of the charge discharging electrode is covered with the insulating layer, the charge discharging electrode is exposed from a bottom surface of the second opening portion, and when a surface of the insulating layer that is in contact with the top surface of the charge discharging electrode is defined as a third surface, and a surface of the insulating layer that is in contact with a portion of the inorganic oxide semiconductor material layer facing the charge accumulation electrode is defined as a second surface, a side surface of the second opening portion has an inclination spreading from the third surface toward the second surface.

Moreover, in the imaging element or the like including the charge discharging electrode according to the present disclosure, it is possible to employ a configuration in which a control unit that is provided in a semiconductor substrate and has a drive circuit is further included, the first electrode, the charge accumulation electrode, and the charge discharging electrode are connected to the drive circuit, a potential V₁₁ is applied to the first electrode and a potential V₃₁ is applied to the charge accumulation electrode and a potential V₆₁ is applied to the charge discharging electrode from the drive circuit, and charge is accumulated in the inorganic oxide semiconductor material layer or the like in the charge accumulation period, and a potential V₁₂ is applied to the first electrode and a potential V₃₂ is applied to the charge accumulation electrode and a potential V₆₂ is applied to the charge discharging electrode from the drive circuit, and the charge accumulated in the inorganic oxide semiconductor material layer or the like is read by the control unit via the first electrode in the charge transfer period.

Here, the potential of the first electrode is higher than the potential of the second electrode, and V₆₁>V₁₁ and V₆₂<V₁₂ are satisfied.

Moreover, in the various preferable embodiment described above of the imaging element or the like including the charge accumulation electrode according to the present disclosure, it is possible to employ a mode in which the charge accumulation electrode is configured of a plurality of charge accumulation electrode segments. The imaging element or the like including the charge accumulation electrode according to the present disclosure in such a mode will be referred to as an “imaging element or the like including a plurality of charge accumulation electrode segments according to the present disclosure” for convenience. It is only necessary for the number of charge accumulation electrode segments may be two or more. Also, in the imaging element or the like including the plurality of charge accumulation electrode segments according to the present disclosure, it is possible to employ a mode in which when a different potential is applied to each of N charge accumulation electrode segments, a potential applied to a charge accumulation electrode segment located at the closest position to the first electrode (the first photoelectric conversion unit segment) is higher than a potential applied to a charge accumulation electrode segment located at the furthest position from the first electrode (N-th photoelectric conversion unit segment) in the charge transfer period when the potential of the first electrode is higher than the potential of the second electrode, and the potential applied to the charge accumulation electrode segment located at the closest position to the first electrode (the first photoelectric conversion unit segment) is lower than the potential applied to the charge accumulation electrode segment located at the furthest position from the first electrode (the N-th photoelectric conversion unit segment) in the charge transfer period when the potential of the first electrode is lower than the potential of the second electrode.

In the imaging element or the like including the charge accumulation electrode according to the present disclosure including the various preferable modes described above, it is possible to employ a configuration in which the semiconductor substrate is provided with at least a floating diffusion layer and an amplification transistor configuring the control unit and the first electrode is connected to the floating diffusion layer and a gate portion of the amplification transistor.

Moreover, in this case, it is possible to employ a configuration in which the semiconductor substrate is further provided with a reset transistor and a selection transistor configuring the control unit, the floating diffusion layer is connected to one source/drain region of the reset transistor, one source/drain region of the amplification transistor is connected to one source/drain region of the selection transistor, and the other source/drain region of the selection transistor is connected to a signal line.

Moreover, in the imaging element including the charge accumulation electrode according to the present disclosure including the various preferable modes described above, it is possible to employ a mode in which the size of the charge accumulation electrode is larger than that of the first electrode. When the area of the charge accumulation electrode is defined as a s₁′, and the area of the first electrode is defined as s₁, 4≤s₁′/s₁ is preferably satisfied although not limited thereto.

Alternatively, modification examples of the imaging element or the like including the charge accumulation electrode according to the present disclosure including the various preferable modes described above can include imaging elements with first to sixth configurations described below. In other words, in the imaging elements with the first to sixth configurations in the imaging element and the like including the charge accumulation electrode according to the present disclosure including the various preferable modes described above, the photoelectric conversion unit is configured of N (where N>2) photoelectric conversion unit segments, the inorganic oxide semiconductor material layer or the like is configured of N photoelectric conversion layer segments, the insulating layer is configured of N insulating layer segments, where in the imaging elements with the first to third configurations, the charge accumulation electrode is configured of N charge accumulation electrode segments, and in the imaging elements with the fourth and fifth configurations, the charge accumulation electrode is configured of N charge accumulation electrode segments disposed to be separated from each other, an n-th (where n=1, 2, 3, . . . N) photoelectric conversion unit segment is configured of an n-th charge accumulation electrode segment, an n-th insulating layer segment, and an n-th photoelectric conversion layer segment, and the photoelectric conversion unit segments with larger n values are located further from the first electrode.

Here, the “photoelectric conversion layer segment” indicates a segment configured of a photoelectric conversion layer and an inorganic oxide semiconductor material layer (and a protective layer) being laminated.

Also, in the imaging element with the first configuration, the thicknesses of the insulating layer segments gradually change from the first photoelectric conversion unit segment to the N-th photoelectric conversion unit segment. Also, in the imaging element with the second configuration, the thicknesses of the photoelectric conversion layer segments gradually change from the first photoelectric conversion unit segment to the N-th photoelectric conversion unit segment. Note that in the photoelectric conversion layer segments, the thicknesses of the photoelectric conversion layer segments may be changed with the thicknesses of portions of the photoelectric conversion layer changed and with the thicknesses of portions of the inorganic oxide semiconductor material layer constantly maintained, or the thicknesses of the photoelectric conversion layer segments may be changed with the thicknesses of portions of the photoelectric conversion layer constantly maintained and with the thicknesses of the portions of the inorganic oxide semiconductor material layer changed, or the thicknesses of the photoelectric conversion layer segments may be changed with the thicknesses of portions of the photoelectric conversion layer changed and with the thicknesses of portions of the inorganic oxide semiconductor material layer changed. Moreover, in the imaging element with the third configuration, materials configuring insulating layer segments are different in adjacent photoelectric conversion unit segments. Also, in the imaging element with the fourth configuration, materials configuring charge accumulation electrode segments are different in adjacent photoelectric conversion unit segments. Moreover, in the imaging element with the fifth configuration, the areas of the charge accumulation electrode segments gradually decreases from the first photoelectric conversion unit segment to the N-th photoelectric conversion unit segment. The areas may continuously decrease or may decrease in a stepwise manner.

Alternatively, in the imaging element with the sixth configuration in the imaging element or the like including the charge accumulation electrode according to the present disclosure including the various preferable modes described above, when the lamination direction of the charge accumulation electrode, the insulating layer, the inorganic oxide semiconductor material layer, and the photoelectric conversion layer is defined as a Z direction, and a direction away from the first electrode is defined as an X direction, the sectional area of the laminated portion in which the charge accumulation electrode, the insulating layer, the inorganic oxide semiconductor material layer, and the photoelectric conversion layer, (and the protective layer) are laminated when the laminated portion is cut along a virtual YZ plane changes depending on the distance from the first electrode. A change in the sectional area may be continuous change or a stepwise change.

In the imaging elements with the first and second configurations, N photoelectric conversion layer segments are continuously provided, N insulating layer segments are also continuously provided, and N charge accumulation electrode segments are also continuously provided. In the imaging elements with the third to fifth configurations, N photoelectric conversion layer segments are continuously provided. Also, in the imaging elements with the fourth and fifth configurations, N insulating layer segments are continuously provided while in the imaging element with the third configuration, N insulating layer segments are provided to correspond to each of the photoelectric conversion unit segments. Moreover, in the imaging elements with the fourth and fifth configurations, and in the imaging element with the third configuration in some cases, N charge accumulation electrode segments are provided to correspond to each of the photoelectric conversion unit segments. In the imaging elements with the first to sixth configurations, the same potential is applied to all the charge accumulation electrode segments. Alternatively, in the imaging elements with the fourth and fifth configurations, and in the imaging element with the third configuration in some cases, different potentials may be applied to each of N charge accumulation electrode segments.

In the imaging element or the like including the charge accumulation electrode according to the present disclosure configured of the imaging elements with the first to sixth configurations, the thickness of the insulating layer segment is defined, or the thickness of the photoelectric conversion layer segment is defined, or materials configuring the insulating layer segments are different, or materials configuring the charge accumulation electrode segments are different, or the area of the charge accumulation electrode segment is defined, or a sectional area of the laminated portion is defined, a kind of charge transfer gradient is thus formed, and it is possible to further easily and reliably transfer charge generated through photoelectric conversion to the first electrode. As a result, it is possible to prevent occurrence of afterimages and occurrence of leaving of charge transfer.

In the imaging elements with the first to fifth configurations, the photoelectric conversion unit segments with larger n values are located further from the first electrode, and whether or not they are located further from the first electrode is determined with reference to an X direction. Also, the direction away from the first electrode in the imaging element with the sixth configuration is defined as the X direction, the “X direction” is defined as follows. In other words, a pixel region in which a plurality of imaging elements or stacked imaging elements are aligned is configured of a plurality of pixels that are regularly aligned in a two-dimensional array shape, that is, in the X direction and a Y direction. In a case in which the planar shape of the pixels is assumed to be rectangular, the direction in which the side that is the closest to the first electrode extends is defined as the Y direction, and the direction that perpendicularly intersects the Y direction is defined as the X direction. Alternatively, in a case in which the planar shape of the pixels is defined as an arbitrary shape, the entire direction including the line segment or the curve that is closest to the first electrode is defined as the Y direction, and the direction that perpendicularly intersects the Y direction is defined as the X direction.

Hereinafter, a case in which the potential of the first electrode is higher than the potential of the second electrode in regard to the imaging elements with the first to sixth configurations will be described.

In the imaging element with the first configuration, the thicknesses of the insulating layer segments gradually change from the first photoelectric conversion unit segment to the N-th photoelectric conversion unit segment, it is preferable that the thicknesses of the insulating layer segments gradually become thicker, and a kind of charge transfer gradient is thus formed. Also, if a state in which V₃₁>V₁₁ is achieved in the charge accumulation period, it is possible to accumulate more charge in the n-th photoelectric conversion unit segment than in the (n+1)-th photoelectric conversion unit segment, a strong field is applied, and it is thus possible to reliably prevent a flow of charge from the first photoelectric conversion unit segment to the first electrode. If a state in which V₃₂<V₁₂ is achieved in the charge transfer period, it is possible to reliably secure a flow of charge from the first photoelectric conversion unit segment to the first electrode and a flow of charge from the (n+1)-th photoelectric conversion unit segment to the n-th photoelectric conversion unit segment.

In the imaging element with the second configuration, the thicknesses of the photoelectric conversion layer segments gradually change from the first photoelectric conversion unit segment to the N-th photoelectric conversion unit segment, it is preferable that the thicknesses of the photoelectric conversion layer segments gradually become thicker, and a kind of charge transfer gradient is thus formed. Also, if a state in which V₃₁>V₁₁ is achieved in the charge accumulation period, a stronger field is applied to the n-th photoelectric conversion unit segment than to the (n+1)-th photoelectric conversion unit segment, and it is possible to reliably prevent a flow of charge from the first photoelectric conversion unit segment to the first electrode. Also, if a state in which V₃₂<V₁₂ is achieved in the charge transfer period, it is possible to reliably secure a flow of charge from the first photoelectric conversion unit segment to the first electrode and a flow of charge from the (n+1)-th photoelectric conversion unit segment to the n-th photoelectric conversion unit segment.

In the imaging element with the third configuration, materials configuring insulating layer segments are different in adjacent photoelectric conversion unit segments, a kind of charge transfer gradient is thus formed, and it is preferable that relative permittivity values of the materials configuring the insulating layer segments gradually become smaller from the first photoelectric conversion unit segment to the N-th photoelectric conversion unit segment. By employing such a configuration, it is possible to accumulate more charge in the n-th photoelectric conversion unit segment than in the (n+1)-th photoelectric conversion unit segment if a state in which V₃₁>V₁₁ is achieved in the charge accumulation period. If a state in which V₃₂<V₁₂ is achieved in the charge transfer period, it is possible to reliably secure a flow of charge from the first photoelectric conversion unit segment to the first electrode and a flow of charge from the (n+1)-th photoelectric conversion unit segment to the n-th photoelectric conversion unit segment

In the imaging element with the fourth configuration, materials configuring charge accumulation electrode segments are different in adjacent photoelectric conversion unit segments, a kind of charge transfer gradient is thus formed, and it is preferable that values of work functions of materials configuring the insulating layer segments gradually become larger from the first photoelectric conversion unit segment to the N-th photoelectric conversion unit segment. By employing such a configuration, it is possible to form a potential gradient that is advantageous for transferring signal charge without depending on positive/negative voltages (potentials).

In the imaging element with the fifth configuration, the areas of the charge accumulation electrode segments gradually become smaller from the first photoelectric conversion unit segment to the N-th photoelectric conversion unit segment, and a kind of charge transfer gradient is thus formed, and it is possible to accumulate more charge in the n-th photoelectric conversion unit segment than in the (n+1)-th photoelectric conversion unit segment if a state in which V₃₁≥V₁₁ is achieved in the charge accumulation period. Also, if a state in which V₃₂<V₁₂ is achieved in the charge transfer period, it is possible to reliably secure a flow of charge from the first photoelectric conversion unit segment to the first electrode and a flow of charge from the (n+1)-th photoelectric conversion unit segment to the n-th photoelectric conversion unit segment.

In the imaging element with a sixth configuration, the sectional area of the laminated portion changes depending on the distance from the first electrode, and a kind of charge transfer gradient is thus formed. Specifically, it is possible to accumulate more charge in a region closer to the first electrode than in a further region if a state in which V₃₁>V₁₁ is achieved in the charge accumulation period similarly to the description of the imaging element with the fifth configuration if a configuration in which the thickness of the section of the laminated portion is set to be constant and the width of the section of the laminated portion is further narrowed away from the first electrode is employed. Therefore, if a state in which V₃₂<V₁₂ is achieved in the charge transfer period, it is possible to reliably secure a flow of charge from the region that is closer to the first electrode to the first electrode and a flow of charge from the further region to the closer region. On the other hand, it is possible to accumulate more charge in the region that is closer to the first electrode than in the further region, a strong field is applied, and it is thus possible to reliably prevent a flow of charge from the region that is closer to the first electrode to the first electrode if V₃₁>V₁₁ is achieved in the charge accumulation period similarly to the description of the imaging element with the first configuration if a configuration in which the width of the section of the laminated portion is set to be constant and the thickness of the section of the laminated portion, specifically, the thickness of the insulating layer segment is set to become gradually thicker is employed. Also, if a state in which V₃₂<V₁₂ is achieved in the charge transfer period, it is possible to reliably secure a flow of charge from the region that is closer to the first electrode to the first electrode and a flow of charge from the further region to the closer region. Also, if a configuration in which the thickness of the photoelectric conversion layer segment is set to gradually become thicker is employed, a stronger field is applied to the region that is closer to the first electrode than to the further region, and it is possible to reliably prevent a flow of charge from the region that is closer to the first electrode to the first electrode if a state in which V₃₁>V₁₁ is achieved in the charge accumulation period similarly to the description of the imaging element with the second configuration. Also, if a state in which V₃₂<V₁₂ is achieved in the charge transfer period, it is possible to reliably secure a flow of charge from the region that is closer to the first electrode to the first electrode and a flow of charge from the further region to the closer region.

Two or more kinds of the imaging elements with the first to sixth configurations including the aforementioned preferable modes can be appropriately combined in accordance with a requirement.

A modification example of the solid-state imaging devices according to the first and second aspects of the present disclosure can be a solid-state imaging device, which includes a plurality of imaging elements with the first to sixth configurations, in which a plurality of imaging elements configure imaging element blocks, and a first electrode is shared by a plurality of imaging elements configuring each imaging element block.

The solid-state imaging device with such a configuration will be referred to as a “solid-state imaging device with a first configuration” for convenience. Alternatively, a modification example of the solid-state imaging devices according to the first and second aspects of the present disclosure can be a solid-state imaging device, which includes a plurality of imaging elements with the first to sixth configurations or stacked imaging elements including at least one imaging element with the first to sixth configurations, in which a plurality of imaging elements or stacked imaging elements configure imaging element blocks, and a first electrode is shared by a plurality of imaging elements or stacked imaging elements configuring each imaging element block.

The solid-state imaging device with such a configuration will be referred to as a “solid-state imaging device with a second configuration” for convenience. If the first electrode is shared by the plurality of imaging elements configuring the imaging element block in this manner, it is possible to simplify and miniaturize a configuration and a structure in a pixel region in which the plurality of imaging elements are aligned.

In the solid-state imaging devices with the first and second configurations, one floating diffusion layer is provided for a plurality of imaging elements (one imaging element block). Here, the plurality of imaging elements provided for one floating diffusion layer may be configured of a plurality of imaging elements of a first type, which will be described later, or may be configured of at least one imaging element of the first type and one or two or more imaging elements of a second type, which will be described later. It is possible for the plurality of imaging elements to share the one floating diffusion layer by appropriately controlling a timing of a charge transfer period. The plurality of imaging elements are caused to operate in conjunction with each other and are connected as an imaging element block to a drive circuit, which will be described later. In other words, the plurality of imaging elements configuring the imaging element block is connected to one drive circuit. However, control of the charge accumulation electrode is performed for each imaging element. Also, it is possible for the plurality of imaging elements to share one contact hole portion. As a disposition relationship between the first electrode shared by the plurality of imaging elements and the charge accumulation electrode of each imaging element, the first electrode may be disposed to be adjacent to the charge accumulation electrode of each imaging element. Alternatively, the first electrode may be disposed to be adjacent to charge accumulation electrodes of a part of the plurality of imaging elements and may be disposed not to be adjacent to the charge accumulation electrodes of remaining ones of the plurality of imaging elements, and in this case, movement of charge from the remaining ones of the plurality of imaging elements to the first electrode becomes movement via the part of the plurality of imaging elements. The distance between a charge accumulation electrode configuring an imaging element and a charge accumulation electrode configuring an imaging element (referred to as a “distance A” for convenience) is preferably longer than the distance between a first electrode and a charge accumulation electrode in an imaging element adjacent to the first electrode (referred to as a “distance B” for convenience) in order to reliably achieve movement of charge from each imaging element to the first electrode. Also, the value of the distance A is preferably set to be larger for the imaging element located further from the first electrode. Note that the above description can be applied not only to the solid-state imaging devices with the first and second configurations but also to the solid-state imaging devices according to the first and second aspects of the present disclosure.

Moreover, in the imaging element or the like including the charge accumulation electrode according to the present disclosure including the various preferable modes described above, it is possible to employ a mode in which light is incident from the side of the second electrode and a light shielding layer is formed to be closer to the light incident side than the second electrode. Alternatively, it is possible to employ a mode in which light is incident from the side of the second electrode and no light is incident on the first electrode (the first electrode and the transfer control electrode in some cases). In this case, it is possible to employ a configuration in which a light shielding layer is formed on the light incident side closer to the second electrode and above the first electrode (the first electrode and the transfer control electrode in some cases), or it is possible to employ a configuration in which an on-chip micro lens is provided above the charge accumulation electrode and the second electrode and light that is incident on the on-chip micro lens is collected by the charge accumulation electrode. Here, the light shielding layer may be disposed above the surface of the second electrode on the light incident side or may be disposed on the surface of the second electrode on the light incident side. In some cases, the light shielding layer may be formed at the second electrode. Examples of a material configuring the light shielding layer includes chromium (Cr), copper (Cu), aluminum (Al), tungsten (W), a resin that does not allow light to pass therethrough (a polyimide resin, for example).

Specific examples of the imaging element or the like including the charge accumulation electrode according to the present disclosure include an imaging element that includes a photoelectric conversion layer or a photoelectric conversion unit absorbing blue light (light of 425 nm to 495 nm) (referred to as a “blue light photoelectric conversion layer of the first type” or a “blue light photoelectric conversion unit of the first type” for convenience) and has sensitivity to blue light (referred to as a “blue light imaging element of the first type” for convenience), an imaging element that includes a photoelectric conversion layer or a photoelectric conversion unit absorbing green light (light of 395 nm to 570 nm) (referred to as a “green light photoelectric conversion layer of the first type” or a “green light photoelectric conversion unit of the first type” for convenience) and has sensitivity to green light (referred to as a “green light imaging element of the first type” for convenience), and an imaging element that includes a photoelectric conversion layer or a photoelectric conversion unit absorbing red light (light of 620 nm to 750 nm) (referred to as a “red light photoelectric conversion layer of the first type” or a “red light photoelectric conversion unit of the first type” for convenience) and has sensitivity to red light (referred to as a “red light imaging element of the first type” for convenience). Also, an imaging element in the related art that does not include any charge accumulation electrode and has sensitivity to blue light will be referred to as a “blue light imaging element of a second type” for convenience, such an imaging element that has sensitivity to green light will be referred to as a “green light imaging element of the second type” for convenience, such an imaging element that has sensitivity to red light will be referred to as a “red light imaging element of the second type” for convenience, a photoelectric conversion layer or a photoelectric conversion unit configuring the blue light imaging element of the second type will be referred to as a “blue light photoelectric conversion layer of the second type” or a “blue light photoelectric conversion unit of the second type” for convenience, a photoelectric conversion layer or a photoelectric conversion unit configuring the green light imaging element of the second type will be referred to as a “green light photoelectric conversion layer of the second type” or a “green light photoelectric conversion unit of the second type” for convenience, and a photoelectric conversion layer or a photoelectric conversion unit configuring the red light imaging element of the second type will be referred to as a “red light photoelectric conversion layer of the second type” or a “red light photoelectric conversion unit of the second type” for convenience.

The stacked imaging element according to the present disclosure includes at least one imaging element or the like (photoelectric conversion element) according to the present disclosure, and specific examples include, for example,

[A] a configuration and a structure in which the blue light photoelectric conversion unit of the first type, the green light photoelectric conversion unit of the first type, and the red light photoelectric conversion unit of the first type are laminated in the vertical direction, and each control unit for the blue light imaging element of the first type, the green light imaging element of the first type, and the red light imaging element of the first type is provided in a semiconductor substrate,
[B] a configuration and a structure in which the blue light photoelectric conversion unit of the first type and the green light photoelectric conversion unit of the first type are laminated in the vertical direction, the red light photoelectric conversion unit of the second type is disposed below the photoelectric conversion units of the first type in these two layers, and each control unit for the blue light imaging element of the first type, the green light imaging element for the first type, and the red light imaging element of the second type is provided in a semiconductor substrate,
[C] a configuration and a structure in which the blue light photoelectric conversion unit of the second type and the red light photoelectric conversion unit of the second type are disposed below the green light photoelectric conversion unit of the first type, and each control unit for the green light imaging element of the first type, the blue light imaging element of the second type, and the red light imaging element of the second type is provided in a semiconductor substrate, and
[D] a configuration and a structure in which the green light photoelectric conversion unit of the second type and the red light photoelectric conversion unit of the second type are disposed below the blue light photoelectric conversion unit of the first type, and each control unit of the blue light imaging element of the first type, the green light imaging element of the second type, and the red light imaging element of the second type is provided in a semiconductor substrate.

The disposition order of the photoelectric conversion units in these imaging elements in the vertical direction is preferably an order of the blue light photoelectric conversion unit, the green light photoelectric conversion unit, and the red light photoelectric conversion unit in the light incident direction or an order of the green light photoelectric conversion unit, the blue light photoelectric conversion unit, and the red light photoelectric conversion unit in the light incident direction. This is because light with a shorter wavelength is more efficiently absorbed on the incident surface side. Since the red color has the longest wavelength among the three colors, the red light photoelectric conversion unit is preferably located in the lowermost layer when seen from the light incident surface. The laminated structure of these imaging elements configures one pixel. Also, a near infrared light photoelectric conversion unit (or an infrared light photoelectric conversion unit) of the first type may be included. Here, a photoelectric conversion layer of the infrared light photoelectric conversion unit of the first type is configured of, for example, an organic material and is preferably disposed in the lowermost layer of the laminated structure of the imaging elements of the first type above the imaging elements of the second type. Alternatively, a near infrared light photoelectric conversion unit (or an infrared light photoelectric conversion unit) of the second type may be included below the photoelectric conversion unit of the first type.

In the imaging element of the first type, a first electrode is formed in an interlayer insulating layer provided on the semiconductor substrate, for example. The imaging element formed on the semiconductor substrate may be of a rear surface irradiation type or a front surface irradiation type.

In a case in which the photoelectric conversion layer is configured of an organic material, the photoelectric conversion layer can be any of the following four aspects.

(1) The photoelectric conversion layer is configured of a p-type organic semiconductor.
(2) The photoelectric conversion layer is configured of an n-type organic semiconductor.
(3) The photoelectric conversion layer is configured of a laminated structure of a p-type organic semiconductor layer/an n-type organic semiconductor layer. The photoelectric conversion layer is configured of a laminated structure of a p-type organic semiconductor layer/a mixture layer of a p-type organic semiconductor and an n-type organic semiconductor (bulk hetero-structure)/an n-type organic semiconductor layer. The photoelectric conversion layer is configured of a laminated structure of a p-type organic semiconductor layer/a mixture layer of a p-type organic semiconductor and an n-type organic semiconductor (bulk hetero-structure). The photoelectric conversion layer is configured of a laminated structure of an n-type organic semiconductor layer/a mixture layer of a p-type organic semiconductor and an n-type organic semiconductor (bulk hetero-structure).
(4) The photoelectric conversion layer is configured of mixture of a p-type organic semiconductor and an n-type organic semiconductor (bulk hetero-structure).

However, it is also possible to employ a configuration in which the order of lamination is arbitrarily changed.

Examples of the p-type organic semiconductor include naphthalene derivatives, anthracene derivatives, phenanthrene derivatives, pyrene derivatives, perylene derivatives, tetracene derivatives, pentacene derivatives, quinacridone derivatives, thiophene derivatives, thienothiophene derivatives, benzothiophene derivatives, benzothienobenzothiophene derivatives, triallylamine derivatives, carbazole derivatives, perylene derivatives, picene derivatives, chrysene derivatives, fluoranthene derivatives, phthalocyanine derivatives, sub-phthalocyanine derivatives, sub-porphyrazine derivatives, metal complexes with heterocyclic compounds as ligands, polythiophene derivatives, polybenzothiadiazole derivatives, polyfluorene derivatives, and the like. Examples of the n-type organic semiconductor include fullerene and fullerene derivatives <for example, fullerenes such as C60, C70, and C74 (higher-order fullerenes), encapsulated fullerenes, and the like) or fullerene derivatives (for example, fullerene fluoride, PCBM fullerene compounds, fullerene multimers, and the like)>, an organic semiconductor having larger (deeper) HOMO and LUMO than those of a p-type organic semiconductor, and a transparent inorganic metal oxide. Specifically, examples of the n-type organic semiconductor include heterocyclic compounds containing nitrogen, oxygen and sulfur atoms, such as organic molecules including pyridine derivatives, pyrazine derivatives, pyrimidine derivatives, triazine derivatives, quinoline derivatives, quinoxaline derivatives, isoquinoline derivatives, acridine derivatives, phenazine derivatives, phenanthroline derivatives, tetrazole derivatives, pyrazole derivatives, imidazole derivatives, thiazole derivatives, oxazole derivatives, imidazole derivatives, benzimidazole derivatives, benzotriazole derivatives, benzoxazole derivatives, benzoxazole derivatives, carbazole derivatives, benzofuran derivatives, dibenzofuran derivatives, subporphyrazine derivatives, polyphenylene vinylene derivatives, polybenzothiadiazole derivatives, polyfluorene derivatives, and the like as a portion of their molecular skeletons, organic metal complexes, and subphthalocyanine derivatives. Examples of a group included in fullerene derivatives include a halogen atom; a straight chain, a branched or cyclic alkyl group or a phenyl group; a group having a straight chain or condensed aromatic compound; a group having a halide; a partial fluoroalkyl group; a perfluoroalkyl group; a silylalkyl group; a silylalkoxy group; an arylsilyl group; an arylsulfanyl group; an alkylsulfanyl group; an arylsulfonyl group; an alkylsulfonyl group; an arylsulfide group; an alkylsulfide group; an amino group; an alkylamino group; an arylamino group; a hydroxy group; an alkoxy group; an acylamino group; an acyloxy group; a carbonyl group; a carboxy group; a carboxoamide group; a carboalkoxy group; an acyl group; a sulfonyl group; a cyano group; a nitro group; a group having a chalcogenide; a phosphine group; a phosphonate group; and derivatives thereof. Although the thickness of the photoelectric conversion layer configured of the organic material (which may be referred to as an “organic photoelectric conversion layer”) is not limited, it is possible to exemplify 1×10⁻⁸ m to 5×10⁻⁷ m, preferably 2.5×10⁻⁸ m to 3×10⁻⁷ m, more preferably 2.5×10⁻⁸ to 2×10⁻⁷ m, and yet more preferably 1×10⁻⁷ m to 1.8×10⁻⁷ m. Note that although an organic semiconductor is often classified into a p-type and an n-type, the p-type means that holes are easily transported, the n-type means that electrons are easily transported, and this is not limited to interpretation that holes or electrons are included as a large number of thermally excited carriers like an inorganic semiconductor.

Additionally, examples of a material configuring an organic photoelectric conversion layer that performs photoelectric conversion of green light include a rhodamine dye, a merocyanine dye, or a quinacridone derivative, a sub-phthalocyanine dye (sub-phthalocyanine derivatives), and the like, examples of a material configuring an organic photoelectric conversion layer that performs photoelectric conversion of blue light include a coumalic acid dye, a tris-8-hydroxyquinoline aluminum (Alq3), a merocyanine dye, and the like, and examples of a material configuring an organic photoelectric conversion layer that performs photoelectric conversion of red light include a phthalocyanine dye and a sub-phthalocyanine dye (sub-phthalocyanine derivatives).

Additionally, examples of an inorganic material configuring the photoelectric conversion layer include crystalline silicon, amorphous silicon, microcrystalline silicon, crystalline selenium, and amorphous selenium, and CIGS (CuInGaSe), CIS (CuInSe₂), CuInS₂, CuAlS₂, CuAlSe₂, CuGaS₂, CuGaSe₂, AgAIS₂, AgAlSe₂, AgInS₂, and AgInSe₂ that are chalcopyrite compounds, and GaAs, InP, AlGaAs, InGaP, AlGaInP, and InGaAsP that are group III-V compounds, and further compound semiconductors such as CdSe, CdS, In₂Se₃, In₂S₃, Bi₂Se₃, Bi₂S₃, ZnSe, ZnS, PbSe, PbS, and the like. In addition, it is also possible to use quantum dots formed of such a material for the photoelectric conversion layer.

The solid-state imaging devices according to the first and second aspects of the present disclosure and the solid-state imaging devices with the first and second configurations can configure a single-plate-type solid-state imaging device.

According to the solid-state imaging device according to the second aspect of the present disclosure including the stacked imaging elements, imaging elements that have sensitivity to light with a plurality of wavelengths are laminated in the light incident direction in the same pixel to configure one pixel unlike a solid-state imaging device including imaging elements in a Bayer layout (that is, without splitting light with a blue color, a green color, and a red color using a color filter layer), and it is thus possible to improve sensitivity and to improve pixel density per unit volume. Also, since the organic material has a higher absorption coefficient, it is possible to reduce the film thickness of the organic photoelectric conversion layer to be thinner than that of an Si-based photoelectric conversion layer in the related art, and leakage of light from adjacent pixels and a limitation of a light incident angle are alleviated. Moreover, although a false color occurs to create color signals by performing complementary processing among pixels of the three colors in the Si-based imaging element in the related art, occurrence of a false color is curbed in the solid-state imaging device according to the second aspect of the present disclosure including the stacked imaging elements. Since the organic photoelectric conversion layer itself also functions as a color filter layer, it is possible to separate colors without disposing the color filter layer.

On the other hand, according to the solid-state imaging device according to the first aspect of the present disclosure, it is possible to alleviate a requirement in regard to properties of splitting light with a blue color, a green color, and a red color and to achieve high mass productivity using a color filter layer. Examples of alignment of the imaging elements in the solid-state imaging device according to the first aspect of the present disclosure include, in addition to the Bayer layout, an interline layout, a G stripe RB checkered layout, G stripe RB complete checkered layout, a checkered complementary color layout, a stripe layout, an oblique stripe layout, a primary color difference layout, a field color difference sequential layout, a frame color difference sequential layout, an MOS-type layout, an improved MOS-type layout, a frame interleaved layout, and a field interleaved layout. Here, one pixel (or a sub-pixel) is configured by one imaging element.

Examples of the color filter layer (wavelength selection means) include a filter layer that allows not only specific wavelengths of a red color, a green color, and a blue color but also specific wavelengths of a cyan color, a magenta color, a yellow color, or the like in some cases to pass therethrough. The color filter layer can be configured not only of an organic material-based color filter layer using an organic compound such as a pigment or a dye but also of a photonic crystal, a thin film formed of an inorganic material of a wavelength selection element to which plasmon is applied (a color filter layer with a conductor grid structure in which a grid-shaped hole structure is provided in a conductor thin film; see JP 2008-177191A, for example), or amorphous silicon.

A pixel region in which a plurality of imaging elements or the like according to the present disclosure or a plurality of stacked imaging elements according to the present disclosure are aligned is configured of a plurality of pixels that are regularly aligned in a two-dimensional array shape. The pixel region is typically configured of an effective pixel region in which light is actually received and signal charge generated through photoelectric conversion is amplified and read in the drive circuit and a black reference pixel region for outputting optical black as a reference of a black level (also referred to as an optical black pixel region (OPB)). The black reference pixel region is typically disposed at an outer peripheral portion of the effective pixel region.

In the imaging element or the like according to the present disclosure including the various preferable modes described above, irradiation with light is performed, photoelectric conversion occurs in the photoelectric conversion layer, and carrier separation of holes and electrons is achieved. An electrode from which the holes are extracted is defined to be a positive pole, and an electrode from which the electrons are extracted is defined to be a negative pole. The first electrode configures the negative pole, and the second electrode configures the positive pole.

The first electrode, the charge accumulation electrode, the transfer control electrode, the charge movement control electrode, the charge discharging electrode, and the second electrode can be configured of a transparent conductive material. The first electrode, the charge accumulation electrode, the transfer control electrode, the charge movement control electrode, and the charge discharging electrode may be collectively referred to as a “first electrode and the like”. Alternatively, in a case in which the imaging elements and the like according to the present disclosure are aligned in a plane as in a Bayer layout, for example, it is possible to employ a configuration in which the second electrode is configured of a transparent conductive material and the first electrode and the like is configured of a metal material, and specifically, it is possible to employ a configuration in which the second electrode located on the light incident side is configured of a transparent conductive material and the first electrode and the like is configured of Al—Nd (an alloy of aluminum and neodymium) or ASC (an alloy of aluminum, samarium, and copper) in this case. The electrode configured of a transparent conductive material may be referred to as a “transparent electrode”. Here, it is desirable that a band gap energy of the transparent conductive material be equal to or greater than 2.5 eV and preferably equal to or greater than 3.1 eV. Examples of a transparent conductive material configuring the transparent electrode include conductive metal oxides, and specific examples thereof include an indium oxide, an indium-tin oxide (including indium tin oxide (ITO), Sn-doped In₂O₃, crystalline ITO, and amorphous ITO), an indium-zinc oxide (IZO) obtained by adding indium as a dopant to a zinc oxide, an indium-gallium oxide (IGO) obtained by adding indium as a dopant to a gallium oxide, an indium-gallium-zinc oxide (IGZO, In—GaZnO₄) obtained by adding indium and gallium as dopants to a zinc oxide, an indium-tin-zinc oxide (ITZO) obtained by adding indium and tin as dopants to a zinc oxide, IFO (F-doped In₂O₃), tin oxide (SnO₂), ATO (Sb-doped SnO₂), FTO (F-doped SnO₂), a zinc oxide (including ZnO doped with another element), an aluminum-zinc oxide (AZO) obtained by adding aluminum as a dopant to a zinc oxide, a gallium-zinc oxide (GZO) obtained by adding gallium as a dopant to zinc oxide, a titanium oxide (TiO₂), a niobium-titanium oxide (TNO) obtained by adding niobium as a dopant to a titanium oxide, antimony oxide, CuI, InSbO₄, ZnMgO, CuInO₂, MgIn₂O₄, CdO, ZnSnO₃, a spinel-type oxide, and an oxide with a YbFe₂O₄ structure. Alternative examples include a transparent electrode containing, as a base layer, a gallium oxide, a titanium oxide, a niobium oxide, a nickel oxide, or the like. Examples of the thickness of the transparent electrode include 2×10⁻⁸ m to 2×10⁻⁷ m or preferably 3×10⁻⁸ m to 1×10⁻⁷ m. In a case in which the first electrode is required to have transparency, the charge discharging electrode is also preferably configured of the transparent conductive material in terms of simplification of the manufacturing process.

Alternatively, in a case in which transparency is not required, the cathode with a function as an electrode from which electrons are extracted is preferably configured of a conductive material with a low work function (t=3.5 eV to 4.5 eV, for example) as a conductive material configuring the same, and specific examples thereof include alkali metal (Li, Na, or K, for example) and fluorides or oxides thereof, alkali earth metal (Mg or Ca, for example) and fluorides or oxides thereof, rare earth metal such as aluminum (Al), zinc (Zn), tin (Sn), thallium (Tl), a sodium-potassium alloy, an aluminum-lithium alloy, a magnesium-silver alloy, indium, and ytterbium, and alloys thereof. Additionally, examples of the material configuring the cathode include metal such as platinum (Pt), gold (Au), palladium (Pd), chromium (Cr), nickel (Ni), aluminum (Al), silver (Ag), tantalum (Ta), tungsten (W), copper (Cu), titanium (Ti), indium (In), tin (Sn), iron (Fe), cobalt (Co), and molybdenum (Mo), alloys containing these metal elements, conductive particles made of such metal, conductive particles of alloys containing such metal, polysilicon containing impurities, carbon materials, oxide semiconductor materials, and conductive materials such as carbon nanotube and graphene, and it is also possible to employ a laminated structure of layers containing these elements. Moreover, examples of the material configuring the cathode include organic materials (conductive polymers) such as poly(3,4-ethylenedioxythiophene)/polystyrene sulfonic acid [PEDOT/PSS]. Also, a paste or an ink obtained by mixing such a conductive material into a binder (polymer) may be hardened and used as an electrode.

As a method for forming the first electrode and the like and the second electrode (a cathode and an anode), it is possible to use a dry method or a wet method. Examples of the dry method include a physical vapor deposition method (PVD method) and a chemical vapor deposition method (CVD method). Examples of a film forming method using the principle of the PVD method include a vacuum vapor deposition method using resistance heating or high frequency heating, an electron beam (EB) deposition method, various sputtering methods (a magnetron sputtering method, an RF-DC coupled bias sputtering method, an ECR sputtering method, an opposed target sputtering method, a high frequency sputtering method, and the like), an ion plating method, a laser ablation method, a molecular beam epitaxy method, and a laser transfer method. Further, examples of the CVD method include a plasma CVD method, a thermal CVD method, an organometallic (MO) CVD method, and an optical CVD method. On the other hand, examples of the wet method include methods such as an electrolytic plating method, an electroless plating method, a spin coating method, an inkjet method, a spray coating method, a stamp method, a micro contact printing method, a flexographic printing method, an offset printing method, a gravure printing method, and a dip method. Examples of a patterning method include chemical etching such as a shadow mask, laser transfer, or photolithography, physical etching using ultraviolet rays, laser, or the like, and the like. As a technology for flattening the first electrode and the like and the second electrode, it is possible to use a laser flattening method, a reflow method, a chemical mechanical polishing (CMP) method, or the like.

Examples of a material configuring the insulating layer include not only inorganic insulating materials including, as examples, metal oxide highly-dielectric insulating material such as silicon oxide materials; silicon nitride (SiN_{Y}); aluminum oxide (Al₂O₃), but also organic insulating materials (organic polymers) including, as examples, polymethyl methacrylate (PMMA); polyvinyl phenol (PVP); polyvinyl alcohol (PVA); polyimide; polycarbonate (PC); polyethylene terephthalate (PET); polystyrene; silanol derivatives (silane coupling agents) such as N-2 (amino ethyl)3-aminopropyl trimethoxysilane (AEAPTMS), 3-mercaptpropyl trimethoxysilane (MPTMS), and octadecyltrichlorosilane (OTS); a novolac-type phenol resin; a fluorine resin; and linear hydrocarbons having, at a terminal, a functional group that can be coupled to a control electrode such as octadecanethiol and dodecyl isocyanate. Examples of the silicon oxide material include silicon oxide (SiOₓ), BPSG, PSG, BSG, AsSG, PbSG, silicon oxynitride (SiON), SOG (spin-on-glass), and low-permittivity insulating materials (polyaryl ether, a cycloperfluorocarbon polymer, benzocyclobutene, a cyclic fluorine resin, polytetrafluoroethylene, aryl ether fluoride, polyimide fluoride, amorphous carbon, and organic SOG, for example). The insulating layer can have a single-layer configuration or can have a configuration in which a plurality of layers (two layers, for example) are laminated. In the latter case, it is only necessary to form an insulating layer as a lower layer at least in a region above the charge accumulation electrode and between the charge accumulation electrode and the first electrode, perform flattening processing on the insulating layer as a lower layer to leave the insulating layer as a lower layer at least in the region between the charge accumulation electrode and the first electrode, and to form an insulating layer as an upper layer above the remaining insulating layer as a lower layer and the charge accumulation electrode, and it is thus possible to reliably achieve flattening of the insulating layer. Materials configuring the protective material layer, various interlayer insulating layers, and the insulating material films may also appropriately selected from these materials.

Configurations and structures of the floating diffusion layer, the amplification transistor, the reset transistor, and the selection transistor configuring the control unit can be similar to the configurations and the structures of a floating diffusion layer, an amplification transistor, a reset transistor, and a selection transistor in the related art. The drive circuit can also have a known configuration and a known structure.

The first electrode is connected to the floating diffusion layer and the gate portion of the amplification transistor, and it is only necessary to form a contact hole portion for connection of the first electrode to the floating diffusion layer and the gate portion of the amplification transistor. Examples of a material configuring the contact hole portion include impurities-doped polysilicon, high-melting-point metal or metal silicide such as tungsten, Ti, Pt, Pd, Cu, TiW, TiN, TiNW, WSi₂, and MoSi₂, and laminated structures (Ti/TiN/W, for example) of layers formed of these materials.

A first carrier blocking layer may be provided between the inorganic oxide semiconductor material layer and the first electrode, and a second carrier blocking layer may be provided between the organic photoelectric conversion layer and the second electrode. Also, a first charge injection layer may be provided between the first carrier blocking layer and the first electrode, and a second charge injection layer may be provided between the second carrier blocking layer and the second electrode. Examples of a material configuring an electron injection layer include alkali metal such as lithium (Li), sodium (Na), and potassium (K) and fluorides and oxides thereof, and alkali earth metal such as magnesium (Mg) and calcium (Ca) and fluorides and oxides thereof.

Examples of a method of forming various organic layers include a dry film formation method and a wet film formation method. Examples of the dry film formation method include a vacuum deposition method using resistance heating, high-frequency heating, or electron beam heating, a flash deposition method, a plasma deposition method, an EB deposition method, various sputtering methods (a two-pole sputtering method, a DC sputtering method, a DC magnetron sputtering method, a high-frequency sputtering method, a magnetron sputtering method, an RF-DC coupled bias sputtering method, an ECR sputtering method, a facing target sputtering method, a high-frequency sputtering method, an ion-beam sputtering method), a direct current (DC) method, an RF method, a multicathode method, an activation reaction method, a field deposition method, various ion plating methods such as a high-frequency ion plating method and a reactive ion plating method, a laser abrasion method, a molecular beam epitaxy method, a laser transfer method, and a molecular beam epitaxy method (MBE method). Also, examples of the CVD method include a plasma CVD method, a thermal CVD method, an MOCVD method, and an optical CVD method. On the other hand, specific examples of the wet method include a spin coating method, a dipping method, a casting method, a micro contact printing method, a drop casting method, various printing methods such as a screen printing method, an inkjet printing method, an offset printing method, a gravure printing method, and a flexographic printing method, a stamp method, a spray method, and various coating methods such as an air doctor coater method, a blade coater method, a rod coater method, a knife coater method, a squeeze coater method, a reverse roll coater method, a transfer roll coater method, a gravure coater method, a kiss coater method, a cast coater method, a spray coater method, a slit orifice coater method, and a calendar coater method. Examples of a solvent in the coating methods include organic solvents with no polarity or a low polarity, such as toluene, chloroform, hexane, and ethanol. Examples of a patterning method include chemical etching such as a shadow mask, a laser transfer, and a photolithography and physical etching using ultraviolet rays, lasers, or the like. As a technology for flattening various organic layers, it is possible to use a laser flattening method, a reflow method, or the like.

The imaging element or the solid-state imaging device may be provided with the on-chip micro lens and the light shielding layer as needed, and the drive circuit and the wiring for driving the imaging element are provided, as described above. A shutter for controlling light that is incident on the imaging element may be disposed as needed, and an optical cut filter may be included in accordance with an application of the solid-state imaging device.

Also, in the solid-state imaging devices with the first and second configurations, it is possible to employ a mode in which one on-chip micro lens is disposed above one imaging element or the like according to the present disclosure, or it is possible to employ a mode in which two imaging elements or the like according to the present disclosure configure an imaging element block and one on-chip micro lens is disposed above the imaging element block.

In a case in which the solid-state imaging device is laminated on a reading integrated circuit (ROIC), for example, it is possible to achieve the lamination by overlapping a drive substrate in which a connecting portion including the reading integrated circuit and copper (Cu) is formed with the imaging element with a connecting portion formed therein such that the connecting portions come into contact with each other and bonding the connecting portions or to bond the connecting portions using a solder bump or the like.

Also, a driving method for driving the solid-state imaging devices according to the first and second aspects of the present disclosure can be a method for driving a solid-state imaging device including: repeating each of steps of discharging charge in the first electrodes to the outside of the system while charge is accumulated in the inorganic oxide semiconductor material layers (or the inorganic oxide semiconductor material layers and the photoelectric conversion layers) at once in all the imaging elements, then transferring the charge accumulated in the inorganic oxide semiconductor material layers (or the inorganic oxide semiconductor material layers and the photoelectric conversion layer) to the first electrodes at once in all the imaging elements, and successively reading the charge transferred to the first electrode in each imaging element after the transfer is completed.

According to such a method for driving a solid-state imaging device, each imaging element has a structure in which light that is incident from the side of the second electrode is not incident on the first electrode, and charge in the first electrode is discharged to the outside of the system while charge is accumulated in the inorganic oxide semiconductor material layer and the like at once in all the imaging elements, and it is thus possible to reliably reset the first electrodes at the same time in all the imaging elements. Thereafter, the charge accumulated in the inorganic oxide semiconductor material layers and the like is transferred to the first electrodes at once in all the imaging elements, and after the transfer is completed, the charge transferred to the first electrode in each imaging element is successively read. It is thus possible to easily realize a so-called global shutter function.

Hereinafter, detailed description of the imaging element and the solid-state imaging device in Example 1 will be described.

The imaging element 10 in Example 1 further includes a semiconductor substrate (more specifically, a silicon semiconductor layer) 70, and the photoelectric conversion layer is disposed above the semiconductor substrate 70. Also, a control unit that is provided in the semiconductor substrate 70 and includes a drive circuit to which the first electrode 21 and the second electrode 22 are connected is further included. Here, a light incident surface of the semiconductor substrate 70 is assumed to be an upper side, and an opposite side of the semiconductor substrate 70 is assumed to be a lower side. On the lower side of the semiconductor substrate 70, a wiring layer 62 configured of a plurality of wirings is provided.

The semiconductor substrate 70 is provided with at least a floating diffusion layer FD₁ and an amplification transistor TR1ₐₘₚ configuring the control unit, and the first electrode 21 is connected to the floating diffusion layer FD₁ and the gate portion of the amplification transistor TR1ₐₘₚ. The semiconductor substrate 70 is further provided with a reset transistor TR1ᵣₛₜ and a selection transistor TR1ₛₑₗ configuring the control unit. The floating diffusion layer FD₁ is connected to one source/drain region of the reset transistor TR1ᵣₛₜ, the other source/drain region of the amplification transistor TR1ₐₘₚ is connected to one source/drain region of the selection transistor TR1ₛₑₗ, and the other source/drain region of the selection transistor TR1ₛₑₗ is connected to the signal line VSL₁. The amplification transistor TR1ₐₘₚ, the reset transistor TR1ᵣₛₜ, and the selection transistor TR1ₛₑₗ configure a drive circuit.

Specifically, the imaging element and the stacked imaging element in Example 1 are an imaging element and a stacked imaging element of a rear surface irradiation type and have a structure in which three imaging elements, namely a green light imaging element in Example 1 of a first type that includes a green light photoelectric conversion layer of the first type absorbing green light and has sensitivity to green light (hereinafter, referred to as a “first imaging element”), a blue light imaging element in the related art of a second type that includes a blue light photoelectric conversion layer of the second type absorbing blue light and has sensitivity to blue light (hereinafter, referred to as a “second imaging element”), and a red light imaging element in the related art of the second type that includes a red light photoelectric conversion layer of the second type absorbing red light and has sensitivity to red light (hereinafter, referred to as a “third imaging element”) are laminated. Here, the red light imaging element (third imaging element) 12 and the blue light imaging element (second imaging element) 11 are provided inside the semiconductor substrate 70, and the second imaging element 11 is located to be closer to the light incident side than the third imaging element 12. Also, the green light imaging element (first imaging element 10) is provided above the blue light imaging element (second imaging element 11). The laminated structure of the first imaging element 10, the second imaging element 11, and the third imaging element 12 configures one pixel. No color filter layer is provided.

In the first imaging element 10, the first electrode 21 and the charge accumulation electrode 24 are formed to be separated from each other above the interlayer insulating layer 81. The interlayer insulating layer 81 and the charge accumulation electrode 24 are covered with the insulating layer 82. The inorganic oxide semiconductor material layer 23B and the photoelectric conversion layer 23A are formed above the insulating layer 82, and the second electrode 22 is formed above the photoelectric conversion layer 23A. The protective material layer 83 is formed over the entire surface including the second electrode 22, and the on-chip micro lens 14 is provided above the protective material layer 83. No color filter layer is provided. The first electrode 21, the charge accumulation electrode 24, and the second electrode 22 are configured of transparent electrodes made of ITO (work function: about 4.4 eV), for example. The inorganic oxide semiconductor material layer 23B contains GaₐSn_{b}Zn_{c}O_{d}. The photoelectric conversion layer 23A is configured of a layer containing a known organic photoelectric conversion material (an organic material such as a rhodamine dye, a merocyanine dye, or quinacridone, for example) that has sensitivity at least to green light. The interlayer insulating layer 81, the insulating layer 82, and the protective material layer 83 are configured of a known insulating material (SiO₂ or SiN, for example). The inorganic oxide semiconductor material layer 23B and the first electrode 21 are connected by the connecting portion 67 provided in the insulating layer 82. The inorganic oxide semiconductor material layer 23B extends inside the connecting portion 67. In other words, the inorganic oxide semiconductor material layer 23B extends inside an opening portion 84 provided in the insulating layer 82 and is connected to the first electrode 21.

The charge accumulation electrode 24 is connected to the drive circuit. Specifically, the charge accumulation electrode 24 is connected to the vertical drive circuit 112 configuring the drive circuit via a connecting hole 66, a pad portion 64, and a wiring V_{OA} provided inside the interlayer insulating layer 81.

The size of the charge accumulation electrode 24 is larger than that of the first electrode 21. When the area of the charge accumulation electrode 24 is defined as s₁′, and the area of the first electrode 21 is defined as s₁, 4≤s₁′/s₁ is preferably satisfied although not limited thereto, and in Example 1, s₁′/s₁=8 is set, for example, although not limited thereto.

An element separation region 71 is formed on the side of the first surface (front surface) 70A of the semiconductor substrate 70, and an oxide film 72 is formed on the first surface 70A of the semiconductor substrate 70. Moreover, the reset transistor TR1ᵣₛₜ, the amplification transistor TR1ₐₘₚ, and the selection transistor TR1ₛₑₗ configuring the control unit of the first imaging element 10 are provided on the side of the first surface of the semiconductor substrate 70, and further, the first floating diffusion layer FD₁ is provided.

The reset transistor TR1ᵣₛₜ is configured of a gate portion 51, a channel formation region 51A, and source/drain regions 51B and 51C. The gate portion 51 of the reset transistor TR1ᵣₛₜ is connected to a reset line RST₁, one source/drain region 51C of the reset transistor TR1ᵣₛₜ also serves as the first floating diffusion layer FD₁, and the other source/drain region 51B is connected to the power source V_{DD}.

The first electrode 21 is connected to the one source/drain region 51C (first floating diffusion layer FD₁) of the reset transistor TR1ᵣₛₜ via the connecting hole 65 and the pad portion 63 provided in the interlayer insulating layer 81, the contact hole portion 61 formed in the semiconductor substrate 70, and the interlayer insulating layer 76, and the wiring layer 62 formed in the interlayer insulating layer 76.

The amplification transistor TR1ₐₘₚ is configured of a gate portion 52, a channel formation region 52A, and source/drain regions 52B and 52C. The gate portion 52 is connected to the first electrode 21 and one source/drain region 51C (first floating diffusion layer FD₁) of the reset transistor TR1ᵣₛₜ via the wiring layer 62. Also, the one source/drain region 52B is connected to the power source V_{DD}.

The selection transistor TR1ₛₑₗ is configured of a gate portion 53, a channel formation region 53A, and source/drain regions 53B and 53C. The gate portion 53 is connected to the selection line SELL Also, one source/drain region 53B shares a region with the other source/drain region 52C configuring the amplification transistor TR1ₐₘₚ, and the other source/drain region 53C is connected to a signal line (data output line) VSL₁ (117).

The second imaging element 11 includes an n-type semiconductor region 41 provided in the semiconductor substrate 70 as a photoelectric conversion layer. A gate portion 45 of a transfer transistor TR2ₜᵣₛ configured of a vertical transistor extends up to the n-type semiconductor region 41 and is connected to a transfer gate line TG₂. Also, the second floating diffusion layer FD₂ is provided in a region 45C of the semiconductor substrate 70 in the vicinity of the gate portion 45 of the transfer transistor TR2ₜᵣₛ. Charge accumulated in the n-type semiconductor region 41 is read in the second floating diffusion layer FD₂ via a transfer channel formed along the gate portion 45.

In the second imaging element 11, a reset transistor TR2ᵣₛₜ, an amplification transistor TR2ₐₘₚ, and a selection transistor TR2ₛₑₗ configuring a control unit of the second imaging element 11 are further provided on the side of the first surface of the semiconductor substrate 70.

The reset transistor TR2ᵣₛₜ is configured of a gate portion, a channel formation region, and source/drain regions. The gate portion of the reset transistor TR2ᵣₛₜ is connected to a reset line RST₂, one source/drain region of the reset transistor TR2ᵣₛₜ is connected to the power source V_{DD}, and the other source/drain region also serves as the second floating diffusion layer FD₂.

The amplification transistor TR2ₐₘₚ is configured of a gate portion, a channel formation region, and source/drain regions. The gate portion is connected to the other source/drain region (second floating diffusion layer FD₂) of the reset transistor TR2ᵣₛₜ. Also, one source/drain region is connected to the power source V_{DD}.

The selection transistor TR2ₛₑₗ is configured of a gate portion, a channel formation region, and source/drain regions. The gate portion is connected to the selection line SEL₂. Also, one source/drain region shares a region with the other source/drain region configuring the amplification transistor TR2ₐₘₚ, and the other source/drain region is connected to a signal line (data output line) VSL₂.

The third imaging element 12 includes an n-type semiconductor region 43 provided in the semiconductor substrate 70 as a photoelectric conversion layer. A gate portion 46 of a transfer transistor TR3ₜᵣₛ is connected to a transfer gate line TGs. Also, a third floating diffusion layer FD₃ is provided in a region 46C of the semiconductor substrate 70 in the vicinity of the gate portion 46 of the transfer transistor TR3ₜᵣₛ. Charge accumulated in the n-type semiconductor region 43 is read in the third floating diffusion layer FD₃ via a transfer channel 46A formed along the gate portion 46.

In the third imaging element 12, a reset transistor TR3ᵣₛₜ, an amplification transistor TR3ₐₘₚ, and a selection transistor TR3ₛₑₗ configuring a control unit of the third imaging element 12 are further provided on the side of the first surface of the semiconductor substrate 70.

The reset transistor TR3ᵣₛₜ is configured of a gate portion, a channel formation region, and source/drain regions. The gate portion of the reset transistor TR3ᵣₛₜ is connected to a reset line RST₃, one source/drain region of the reset transistor TR3ᵣₛₜ is connected to the power source V_{DD}, and the other source/drain region also serves as the third floating diffusion layer FD₃.

The amplification transistor TR3ₐₘₚ is configured of a gate portion, a channel formation region, and source/drain regions. The gate portion is connected to the other source/drain region (third floating diffusion layer FD₃) of the reset transistor TR3ᵣₛₜ. Also, one source/drain region is connected to the power source V_{DD}.

The selection transistor TR3ₛₑₗ is configured of a gate portion, a channel formation region, and source/drain regions. The gate portion is connected to the selection line SEL₃. Also, one source/drain region shares a region with the other source/drain region configuring the amplification transistor TR3ₐₘₚ, and the other source/drain region is connected to a signal line (data output line) VSL₃.

The reset lines RST₁, RST₂, and RST₃, the selection lines SEL₁, SEL₂, and SEL₃, and the transfer gate lines TG₂ and TG₃ are connected to the vertical drive circuit 112 configuring the drive circuit, and the signal lines (data output lines) VSL₁, VSL₂, and VSL₃ are connected to the column signal processing circuit 113 configuring the drive circuit.

A p⁺ layer 44 is provided between the n-type semiconductor region 43 and the front surface 70A of the semiconductor substrate 70 to curb occurrence of a dark current. A p⁺ layer 42 is formed between the n-type semiconductor region 41 and the n-type semiconductor region 43, and further, a part of a side surface of the n-type semiconductor region 43 is surrounded by the p⁺ layer 42. A p⁺ layer 73 is formed on the side of the rear surface 70B of the semiconductor substrate 70, and an HfO₂ film 74 and an insulating material film 75 are formed at a portion where the contact hole portion 61 is to be formed inside the semiconductor substrate 70 from the p⁺ layer 73. Although the interlayer insulating layer 76 includes a wiring formed across a plurality of layers, illustration thereof is omitted.

The HfO₂ film 74 is a film with negative fixed charge, and it is possible to curb occurrence of a dark current by providing such a film. It is also possible to use, instead of the HfO₂ film, an aluminum oxide (Al₂O₃) film, a zirconium oxide (ZrO₂) film, a tantalum oxide (Ta₂O₅) film, a titanium oxide (TiO₂) film, a lanthanum oxide (La₂O₃) film, a praseodymium oxide (Pr₂O₃) film, a cerium oxide (CeO₂) film, a neodyminum oxide (Nd₂O₃) film, a promethium oxide (Pm₂O₃) film, a samarium oxide (Sm₂O₃) film, an europium oxide (Eu₂O₃) film, a gadolinium oxide (Gd₂O₃) film, a terbium oxide (Tb₂O₃) film, a dysprosium oxide (Dy₂O₃) film, a holmium oxide (Ho₂O₃) film, a thulium oxide (Tm₂O₃) film, an ytterbium oxide (Yb₂O₃) film, a lutetium oxide (Lu₂O₃) film, an yttrium oxide (Y₂O₃) film, a hafnium nitride film, an aluminum nitride film, a hafnium oxynitride film, or an aluminum oxynitride film. As a method for forming such a film, it is possible to exemplify a CVD method, a PVD method, and an ALD method, for example.

Hereinafter, operations of the stacked imaging element (first imaging element 10) including the charge accumulation electrode in Example 1 will be described with reference to FIGS. 5 and 6A. The imaging element in Example 1 is provided in the semiconductor substrate 70 and further includes a control unit including a drive circuit, and the first electrode 21, the second electrode 22, and the charge accumulation electrode 24 are connected to the drive circuit. Here, the potential of the first electrode 21 is set to be higher than the potential of the second electrode 22. In other words, the first electrode 21 is caused to have a positive potential, the second electrode 22 is caused to have a negative potential, and electrons generated through photoelectric conversion in the photoelectric conversion layer 23A are read in the floating diffusion layer. The same applies to the other examples.

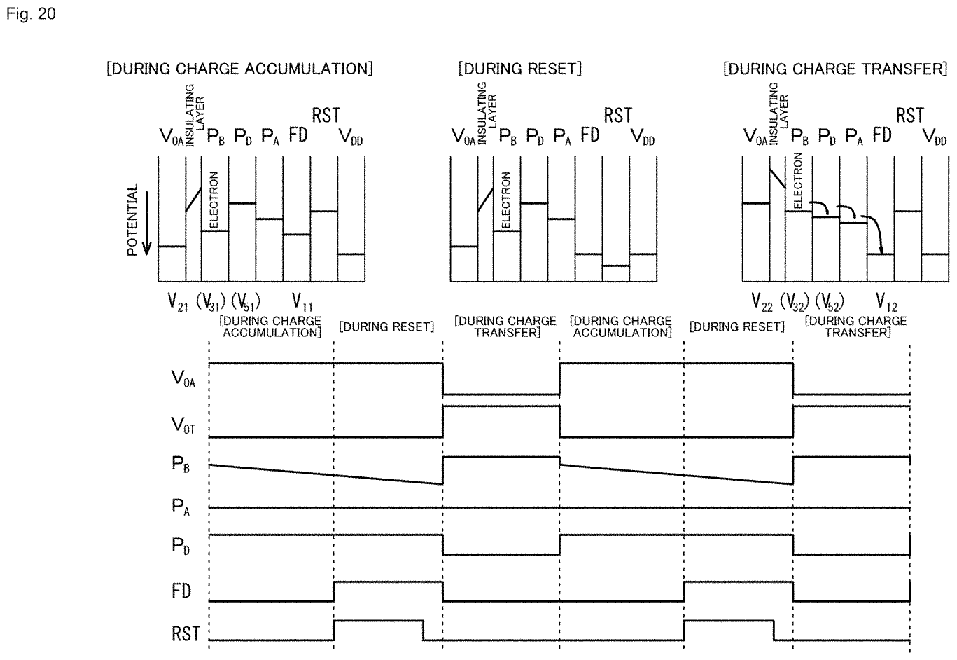

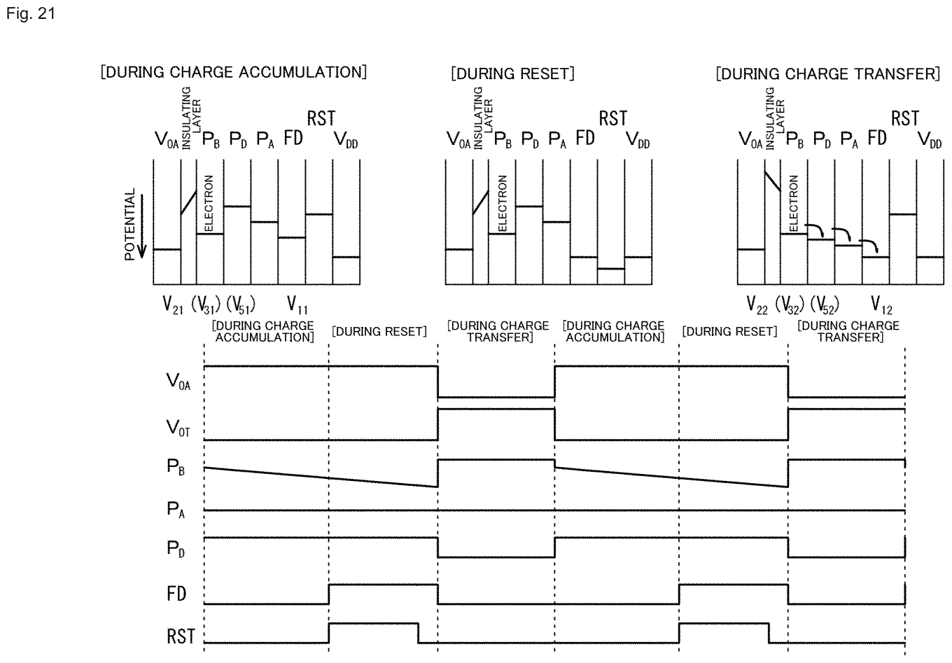

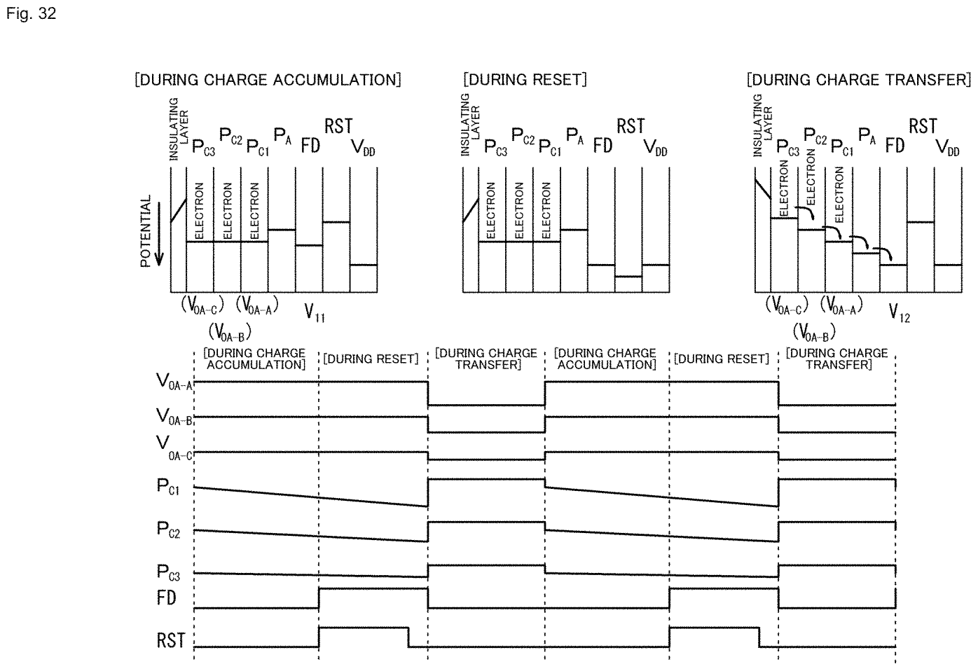

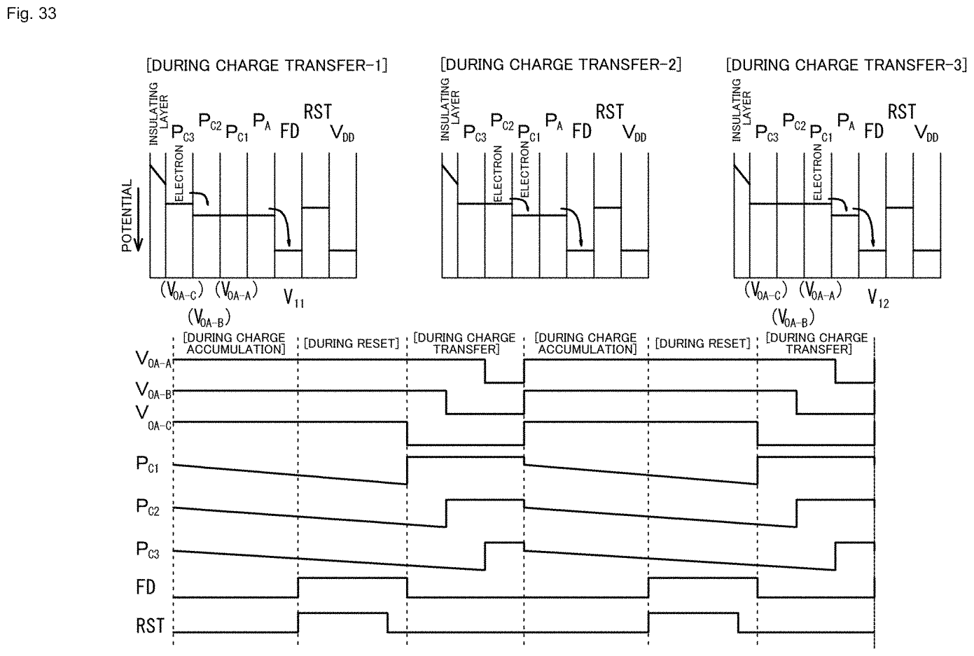

The signs used in FIG. 5, FIGS. 20 and 21 in Example 4, and FIGS. 32 and 33 in Example 6, which will be described later, are as follows.

P_{A} . . . A potential at the point P_{A} in a region of the inorganic oxide semiconductor material layer 23B facing a region located at a midpoint between the charge accumulation electrode 24 or the transfer control electrode (charge transfer electrode) 25 and the first electrode 21
P_{B} . . . A potential at the point P_{B} in a region of the inorganic oxide semiconductor material layer 23B facing the charge accumulation electrode 24
P_{C1} . . . A potential at the point P_{C1} in a region of the inorganic oxide semiconductor material layer 23B facing the charge accumulation electrode segment 24A
P_{C2} . . . A potential at the point P_{C2} in a region of the inorganic oxide semiconductor material layer 23B facing the charge accumulation electrode segment 24B
P_{C3} . . . A potential at the point P_{C3} in a region of the inorganic oxide semiconductor material layer 23B facing the charge accumulation electrode segment 24C
P_{D} . . . A potential at the point P_{D} in a region of the inorganic oxide semiconductor material layer 23B facing the transfer control electrode (charge transfer electrode) 25
FD . . . A potential in the first floating diffusion layer FD₁
V_{OA} . . . A potential in the charge accumulation electrode 24
V_{OA-A} . . . A potential in the charge accumulation electrode segment 24A
V_{OA-B} . . . A potential in the charge accumulation electrode segment 24B
V_{OA-C} . . . A potential in the charge accumulation electrode segment 24C
V_{OT} . . . A potential in the transfer control electrode (charge transfer electrode) 25
RST . . . A potential in the gate portion 51 of the reset transistor TR1ᵣₛₜ
V_{DD} . . . A potential of power source
VSL₁ . . . Signal line (data output line) VSL₁
TR1ᵣₛₜ . . . Reset transistor TR1ᵣₛₜ
TR1ₐₘₚ . . . Amplification transistor TR1ₐₘₚ
TR1ₛₑₗ . . . Selection transistor TR1ₛₑₗ

In the charge accumulation period, a potential V₁₁ is applied to the first electrode 21, and a potential V₃₁ is applied to the charge accumulation electrode 24, from the drive circuit. Photoelectric conversion occurs in the photoelectric conversion layer 23A due to light that is incident on the photoelectric conversion layer 23A. Holes generated through photoelectric conversion are fed from the second electrode 22 to the drive circuit via a wiring V_{OU}. On the other hand, since the potential of the first electrode 21 is set to be higher than the potential of the second electrode 22, that is, a positive potential is applied to the first electrode 21, and a negative potential is applied to the second electrode 22, for example, V₃₁>V₁₁ or preferably V₃₁>V₁₁ is set. In this manner, electrons generated through photoelectric conversion are attracted by the charge accumulation electrode 24 and stop at a region of the inorganic oxide semiconductor material layer 23B facing the charge accumulation electrode 24 or the inorganic oxide semiconductor material layer 23B and the photoelectric conversion layer 23A (hereinafter, these will be collectively referred to as an “inorganic oxide semiconductor material layer 23B and the like”). In other words, charge is accumulated in the inorganic oxide semiconductor material layer 23B and the like. Since V₃₁>V₁₁, electrons generated inside the photoelectric conversion layer 23A do not move toward the first electrode 21. With elapse of time of the photoelectric conversion, the potential in the region of the inorganic oxide semiconductor material layer 23B and the like facing the charge accumulation electrode 24 becomes a value that is yet closer to the negative side.

In the latter half of the charge accumulation period, a reset operation is performed. In this manner, the potential of the first floating diffusion layer FD₁ is reset, and the potential of the first floating diffusion layer FD₁ becomes a potential V_{DD} of a power source.

After the reset operation is completed, reading of charge is performed. In other words, a potential V₁₂ is applied to the first electrode 21, and a potential V₃₂ is applied to the charge accumulation electrode 24, from the drive circuit in the charge transfer period. Here, V₃₂<V₁₂ is set. In this manner, electrons stopping at the region of the inorganic oxide semiconductor material layer 23B and the like facing the charge accumulation electrode 24 are read in the first electrode 21 and further the first floating diffusion layer FD₁. In other words, the charge accumulated in the inorganic oxide semiconductor material layer 23B and the like is read by the control unit.

As described above, a series of operations including the charge accumulation, the reset operation, and the charge transfer are completed.

Operations of the amplification transistor TR1ₐₘₚ and the selection transistor TR1ₛₑₗ after the electrons are read in the first floating diffusion layer FD₁ are the same as the operations of these transistors in the related art. Also, a series of operations including charge accumulation, reset operations, and charge transfer of the second imaging element 11 and the third imaging element 12 is similar to a series of operations including charge accumulation, a reset operation, and charge transfer in the related art. Also, reset noise of the first floating diffusion layer FD₁ can be removed through correlated double sampling (CDS) processing similarly to the related art.

As described above, since the charge accumulation electrode that is disposed to be separated from the first electrode and is disposed to face the photoelectric conversion layer via an insulating layer is included in Example 1, a kind of capacitor is formed by the inorganic oxide semiconductor material layer and the like, the insulating layer, and the charge accumulation electrode, and it is possible to accumulate charge in the inorganic oxide semiconductor material layer and the like when the photoelectric conversion layer is irradiated with light and photoelectric conversion is performed in the photoelectric conversion layer. It is thus possible to achieve full depletion of the charge accumulation unit and cancel out charge when the exposure is started. As a result, it is possible to curb occurrence of the phenomenon that kTC noise increases, random noise deteriorates, and this leads to degradation of quality of captured images. Also, it is possible to reset all the pixels at once and thereby to realize a so-called global shutter function.

FIG. 68 illustrates a conceptual diagram of the solid-state imaging device in Example 1. A solid-state imaging device 100 in Example 1 is configured of an imaging region 111 in which stacked imaging elements 101 are aligned in a two-dimensional array, the vertical drive circuit 112, the column signal processing circuit 113, the horizontal drive circuit 114, the output circuit 115, the drive control circuit 116, and the like as drive circuits (peripheral circuits) thereof. It is needless to say that these circuits can be configured of known circuits or can be configured using other circuit configurations (for example, various circuits used in a CCD imaging device or a CMOS imaging device in the related art). In FIG. 68, the reference sign “101” of the stacked imaging element 101 is illustrated only for one row.

The drive control circuit 116 generates clock signals and control signals as references for operations of the vertical drive circuit 112, the column signal processing circuit 113, and the horizontal drive circuit 114 on the basis of vertical synchronization signals, horizontal synchronization signals, and a master clock. Then, the generated clock signals and control signals are input to the vertical drive circuit 112, the column signal processing circuit 113, and the horizontal drive circuit 114.

The vertical drive circuit 112 is configured of a shift register, for example, and sequentially and selectively scans each stacked imaging element 101 in the imaging region 111 in the vertical direction in units of rows. Then, a pixel signal (image signal) based on a current (signal) generated in accordance with the amount of light received in each stacked imaging element 101 is sent to the column signal processing circuit 113 via signal lines (data output lines) 117 and VSL.

The column signal processing circuit 113 is disposed for each column of the stacked imaging elements 101, for example, and signal processing such as noise removal and signal amplification is performed on the image signals output from the stacked imaging elements 101 corresponding to one row using signals from black reference pixels (formed in the surroundings of an effective pixel region although it is not illustrated) for each imaging element. In an output level of the column signal processing circuit 113, a horizontal selection switch (not illustrated) is provided to be connected to a horizontal signal line 118.

The horizontal drive circuit 114 is configured of, for example, a shift register, sequentially outputs horizontal scanning pulses and thus sequentially selects each of the column signal processing circuits 113, and outputs signals from each of the column signal processing circuits 113 to the horizontal signal line 118.

The output circuit 115 performs signal processing on signals that are sequentially supplied through the horizontal signal line 118 from each column signal processing circuit 113, and outputs the processed signals.

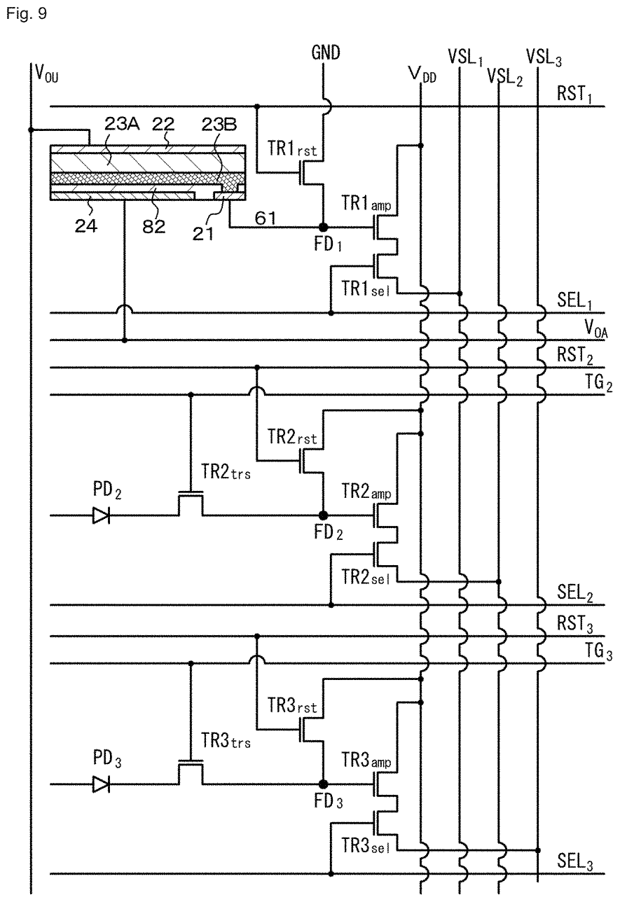

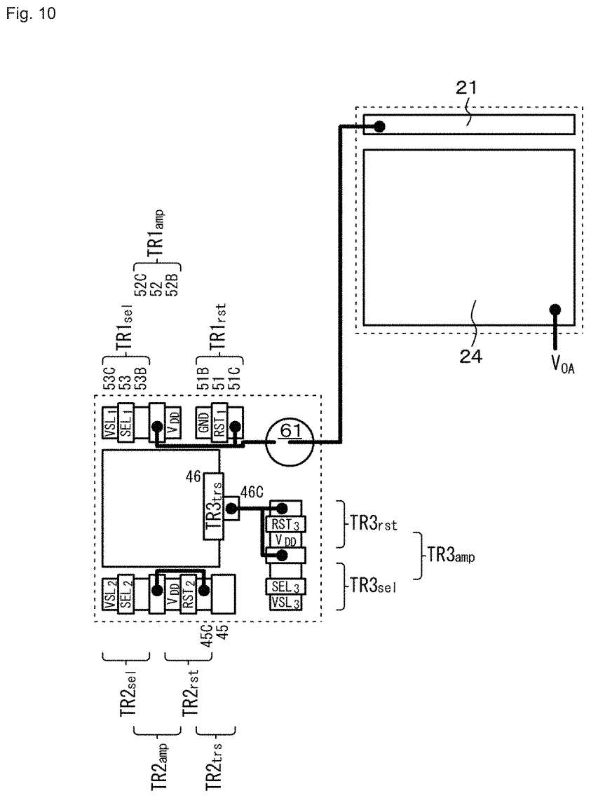

As illustrated in FIG. 9 as an equivalent circuit diagram of a modification example of the imaging element and a stacked imaging element in Example 1 and in FIG. 10 as a schematic disposition diagram of the first electrode and the charge accumulation electrode and the transistors configuring the control unit, the other source/drain region 51B of the reset transistor TR1ᵣₛₜ may be grounded instead of being connected to the power source V_{DD}.

The imaging element and the stacked imaging element in Example 1 can be produced by the following method, for example. In other words, an SOI substrate is prepared first. Then, a first silicon layer is formed on the surface of the SOI substrate by an epitaxial growth method, and the p⁺ layer 73 and the n-type semiconductor region 41 are formed on the first silicon layer. Next, a second silicon layer is formed on the first silicon layer by the epitaxial growth method, and the element separation region 71, the oxide film 72, the p⁺ layer 42, the n-type semiconductor region 43, and the p⁺ layer 44 are formed on the second silicon layer. Also, various transistors and the like configuring the control unit of the imaging element are formed on the second silicon layer, the wiring layer 62, the interlayer insulating layer 76, and various wirings are further formed thereon, and the interlayer insulating layer 76 and a support substrate (not illustrated) are attached. Thereafter, the SOI substrate is removed to cause the first silicon layer to be exposed. The front surface of the second silicon layer corresponds to the front surface 70A of the semiconductor substrate 70, and the front surface of the first silicon layer corresponds to the rear surface 70B of the semiconductor substrate 70. Also, the first silicon layer and the second silicon layer are collectively expressed as the semiconductor substrate 70. Then, an opening portion for forming the contact hole portion 61 is formed on the side of the rear surface 70B of the semiconductor substrate 70, the HfO₂ film 74, the insulating material film 75, and the contact hole portion 61 are formed, and the pad portions 63 and 64, the interlayer insulating layer 81, the connecting holes 65 and 66, the first electrode 21, the charge accumulation electrode 24, and the insulating layer 82 are further formed. Next, the connecting portion 67 is opened, and the inorganic oxide semiconductor material layer 23B, the photoelectric conversion layer 23A, the second electrode 22, the protective material layer 83, and the on-chip micro lens 14 are formed. As described above, it is possible to obtain the imaging element and the stacked imaging element in Example 1.

Although not illustrated in the drawing, the insulating layer 82 may have a two-layer configuration of an insulating layer as a lower layer and an insulating layer as an upper layer. In other words, it is only necessary to form the insulating layer as a lower layer in at least a region above the charge accumulation electrode 24 and between the charge accumulation electrode 24 and the first electrode 21 (more specifically, form the insulating layer as a lower layer above the interlayer insulating layer 81 including the charge accumulation electrode 24), to perform flattening on the insulating layer as a lower layer, and to form the insulating layer as an upper layer above the insulating layer as a lower layer and the charge accumulation electrode 24, and it is thus possible to reliably flatten the insulating layer 82. It is only necessary to open the connecting portion 67 in the thus obtained insulating layer 82.

### Example 2

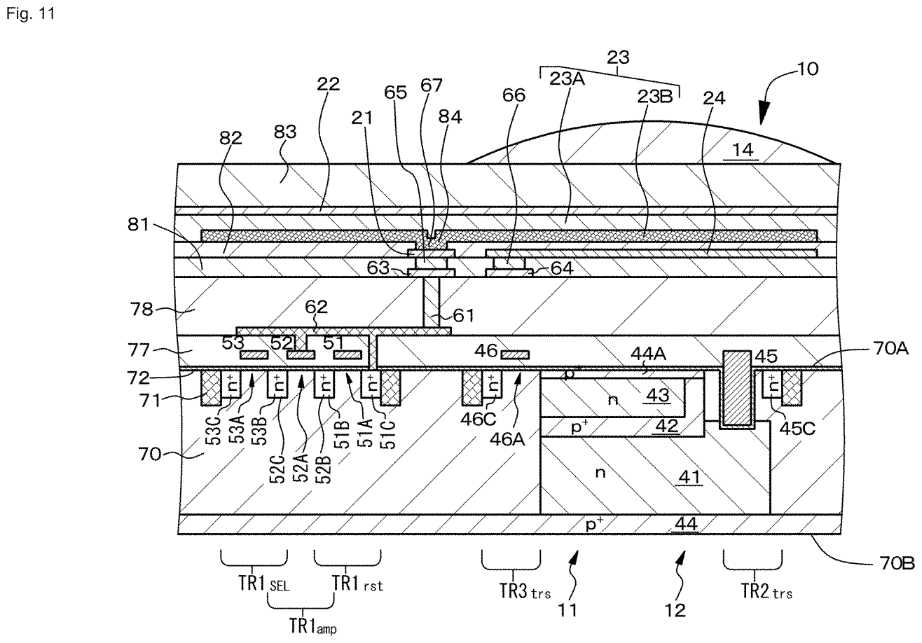

Example 2 is a modification of Example 1. An imaging element and a stacked imaging element in Example 2 illustrated in FIG. 11 as a schematic partial sectional view are an imaging element and a stacked imaging element of a front surface irradiation type and have a structure in which three imaging elements, namely a green light imaging element (first imaging element 10) in Example 1 of a first type that includes a green light photoelectric conversion layer of the first type absorbing green light and has sensitivity to green light, a blue light imaging element (second imaging element 11) in the related art of a second type that includes a blue light photoelectric conversion layer of the second type absorbing blue light and has sensitivity to blue light, and a red light imaging element (third imaging element 12) in the related art of the second type that includes a red light photoelectric conversion layer of the second type absorbing red light and has sensitivity to red light are laminated. Here, the red light imaging element (third imaging element 12) and the blue light imaging element (second imaging element 11) are provided inside a semiconductor substrate 70, and the second imaging element 11 is located on a side closer to a light incident side than the third imaging element 12. Also, the green light imaging element (first imaging element 10) is provided above the blue light imaging element (second imaging element 11).

Various transistors configuring a control unit are provided on a side of a front surface 70A of the semiconductor substrate 70 similarly to Example 1. These transistors can have configurations and structures that are similar to those of the transistors described in Example 1. The semiconductor substrate 70 is provided with the second imaging element 11 and the third imaging element 12, and these imaging elements can have configurations and structures that are substantially similar to those of the second imaging element 11 and the third imaging element 12 described in Example 1.

The interlayer insulating layer 81 is formed above the front surface 70A of the semiconductor substrate 70, and the first electrode 21, the inorganic oxide semiconductor material layer 23B, the photoelectric conversion layer 23A, the second electrode 22, the charge accumulation electrode 24, and the like are provided above the interlayer insulating layer 81 similarly to the imaging element in Example 1.

In this manner, the configurations and the structures of the imaging element and the stacked imaging element in Example 2 can be similar to the configurations and the structures of the imaging element and the stacked imaging element in Example 1 other than that the imaging element and the stacked imaging element in Example 2 are of the front surface irradiation type, and detailed description will thus be omitted.

### Example 3

Example 3 is a modification of Examples 1 and 2.

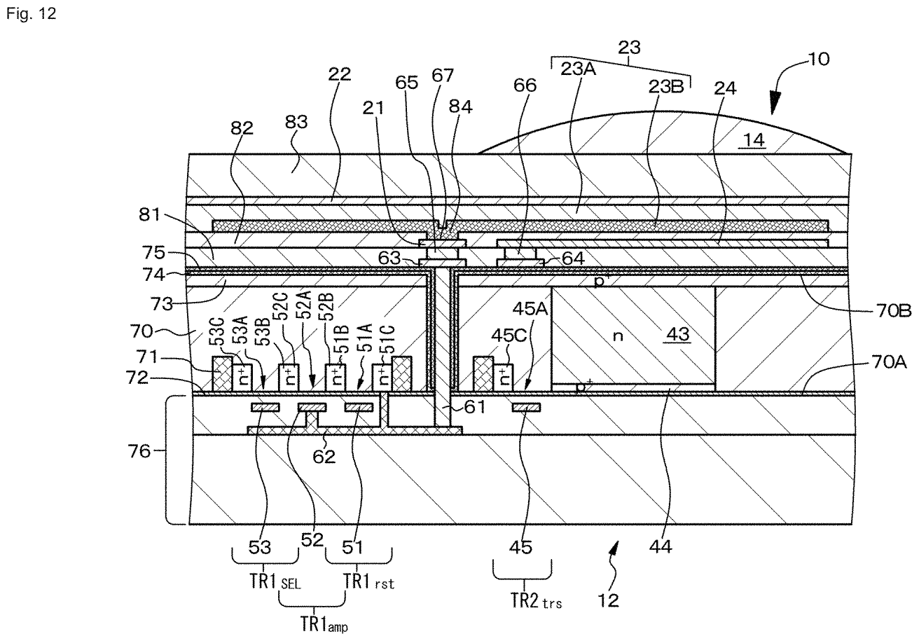

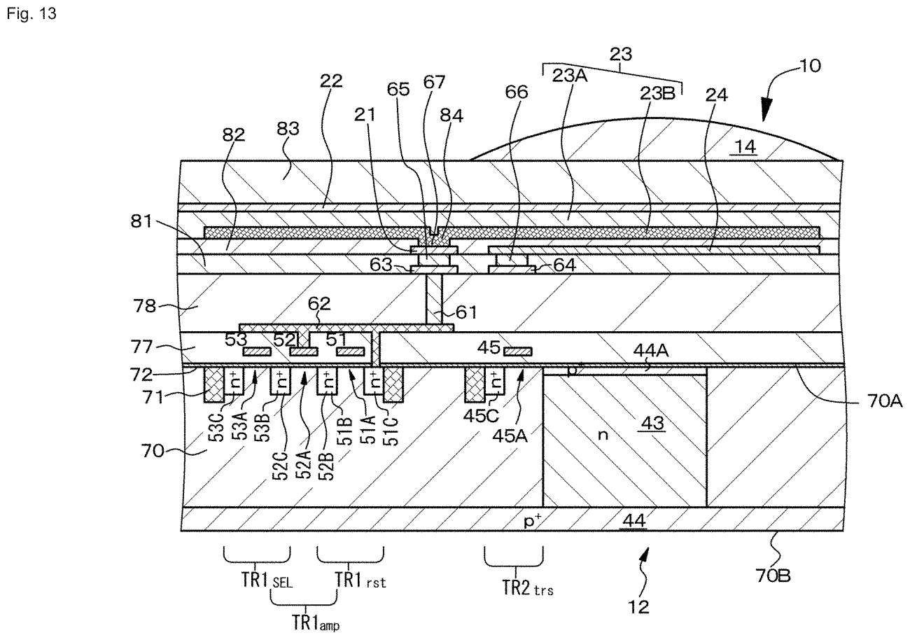

An imaging element and a stacked imaging element in Example 3 illustrated in FIG. 12 as a schematic partial sectional view are an imaging element and a stacked imaging element of a rear surface irradiation type and have a structure in which two imaging elements, namely the first imaging element 10 in Example 1 of a first type and the third imaging element 12 of a second type are laminated. A modification example of the imaging element and the stacked imaging element in Example 3 illustrated in FIG. 13 as a schematic partial sectional view relates to an imaging element and a stacked imaging element of a front surface irradiation type and have a structure in which two imaging elements, namely the first imaging element 10 in Example 1 of the first type and the third imaging element 12 of the second type are laminated. Here, the first imaging element 10 absorbs light of a primary color, and the third imaging element 12 absorbs light of a complementary color. Alternatively, the first imaging element 10 absorbs white light, and the third imaging element 12 absorbs infrared rays.

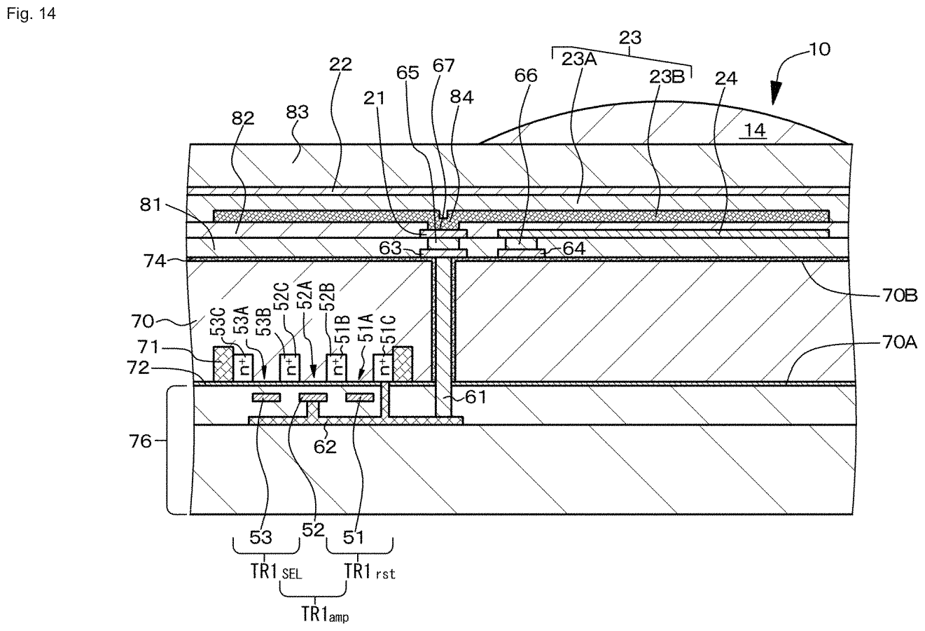

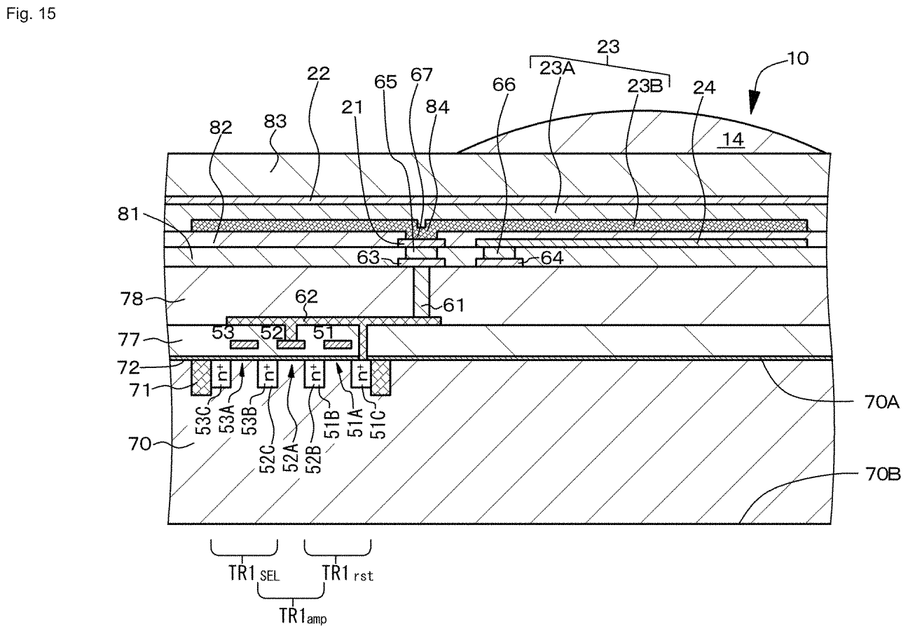

A modification example of the imaging element in Example 3 illustrated in FIG. 14 as a schematic partial sectional view relates to an imaging element of a rear surface irradiation type and is configured of the first imaging element 10 in Example 1 of the first type. Also, a modification example of the imaging element in Example 3 illustrated in FIG. 15 as a schematic partial sectional view relates to an imaging element of a front surface irradiation type and is configured of the first imaging element 10 in Example 1 of the first type. Here, the first imaging element 10 is configured of three kinds of imaging elements, namely an imaging element that absorbs red light, an imaging element that absorbs green light, and an imaging element that absorbs blue light. Moreover, a plurality of such imaging elements configure a solid-state imaging device according to a first aspect of the present disclosure. As layout of such a plurality of imaging elements, it is possible to exemplify Bayer layout. A color filter layer for separating blue, green, and red light is disposed as needed on a light incident side of each imaging element.

It is also possible to employ a mode in which two imaging elements in Example 1 of the first type are laminated (that is, a mode in which two photoelectric conversion units are laminated and a control unit for the two photoelectric conversion unit is provided in a semiconductor substrate) or a mode in which three imaging elements in Example 1 of the first type are laminated (that is, a mode in which three photoelectric conversion units are laminated and a control unit for the three photoelectric conversion units is provided in a semiconductor substrate), instead of providing one imaging element in Example 1 of the first type. Laminated structure examples of the imaging element of the first type and the imaging element of the second type are illustrated in the table below.

| | First type | Second type |
|---|---|---|
| Rear | 1 | 2 |
| surface | Green | Blue color + red color |
| irradiation | 1 | 1 |
| type | Primary color | Complementary color |
| and font | 1 | 1 |
| surface | White color | Infrared ray |
| irradiation | 1 | 0 |
| type | Blue color, green color, | |
| | or red coor | |
| | 2 | 2 |
| | Green color + infrared light | Blue color + red color |
| | 2 | 1 |
| | Green color + blue color | Red color |
| | 2 | 0 |
| | White color + infrared light | |
| | 3 | 2 |
| | Green color + blue color + | Blue green odor |
| | red color | (emerald color) + |
| | | infrared light |
| | 3 | 1 |
| | Green color + blue color + | Infrared light |
| | red color | |
| | 3 | 0 |
| | Blue color + green color + | |
| | red color | |

### Example 4

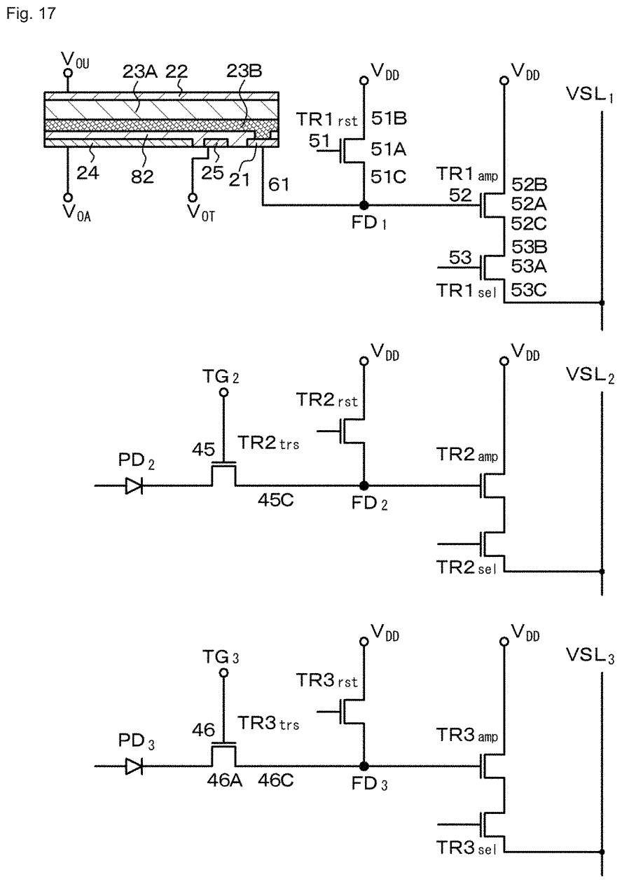

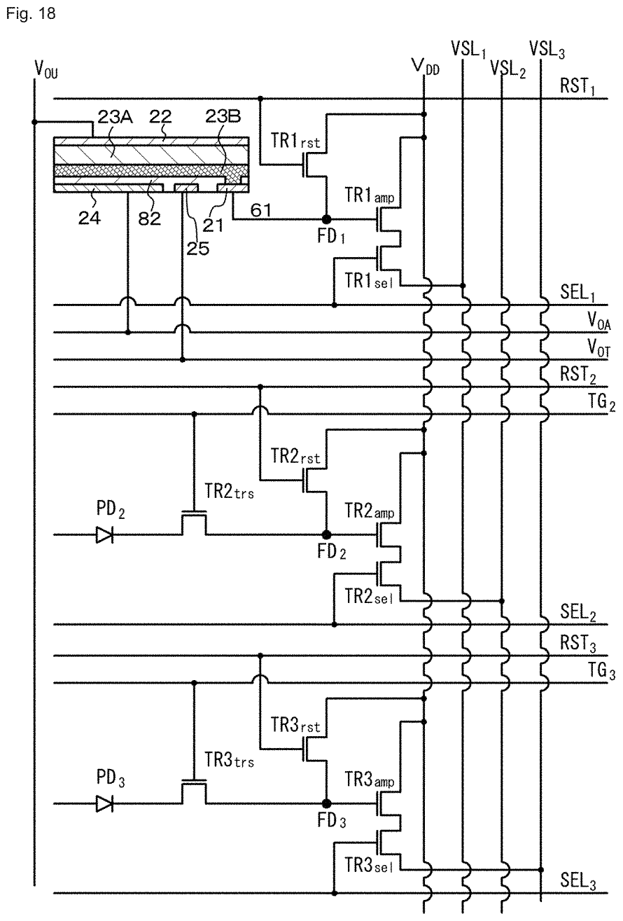

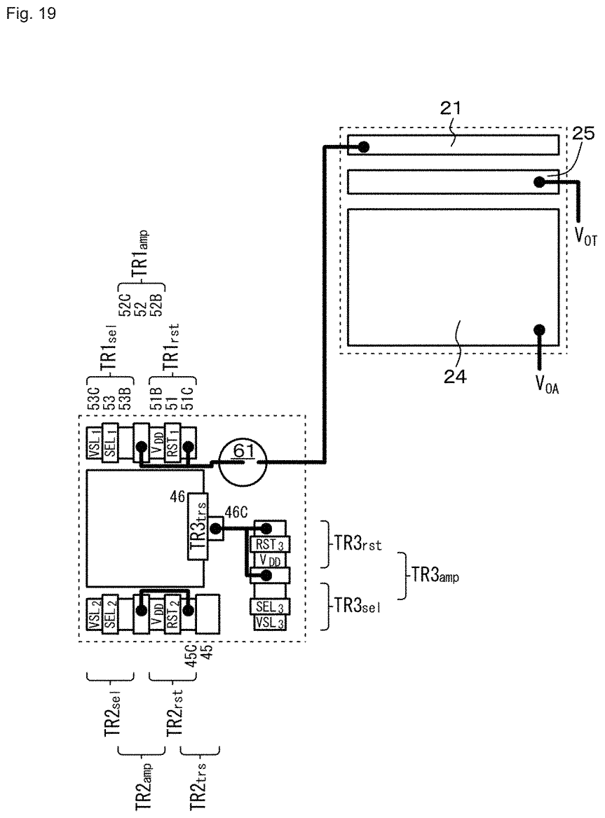

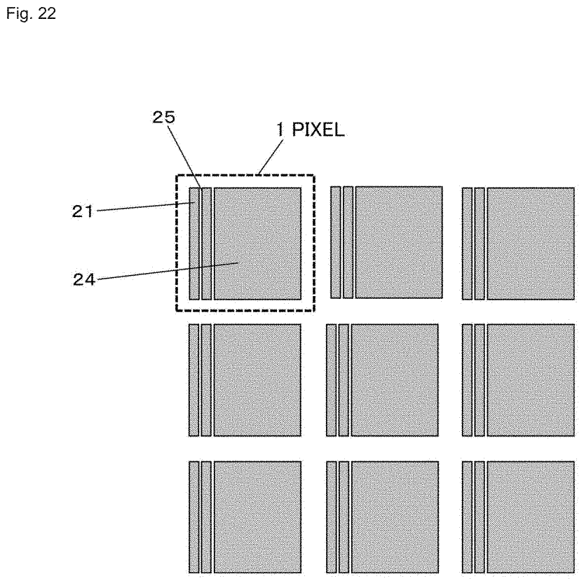

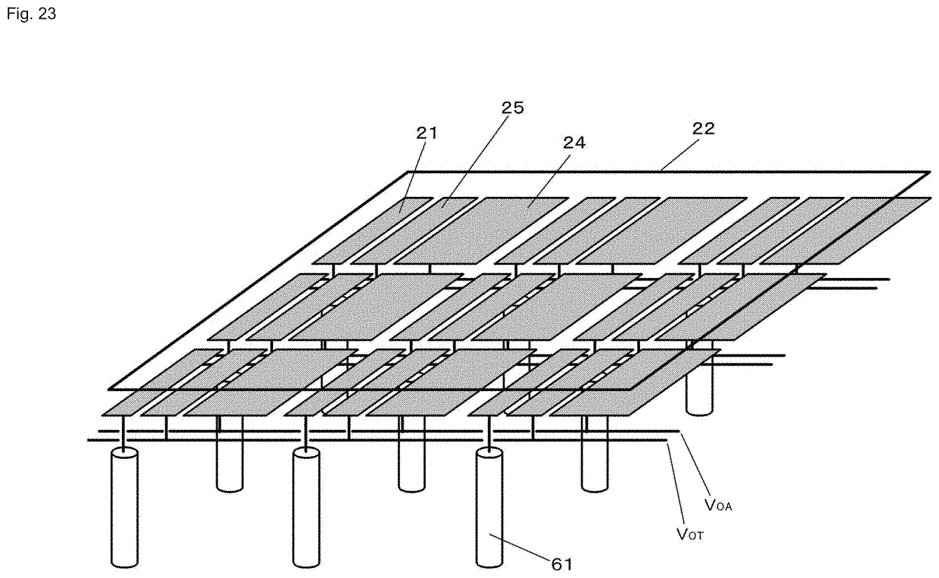

Example 4 is a modification of Examples 1 to 3 and relates to an imaging element or the like including the transfer control electrode (charge transfer electrode) according to the present disclosure. A schematic partial sectional view of a portion of an imaging element and a stacked imaging element in Example 4 is illustrated in FIG. 16, equivalent circuit diagrams of the imaging element and the stacked imaging element in Example 4 are illustrated in FIGS. 17 and 18, a schematic disposition diagram of a first electrode, a transfer control electrode, and a charge accumulation electrode configuring the imaging element in Example 4 and transistors configuring a control unit is illustrated in FIG. 19, states of a potential at each portion when the imaging element in Example 4 is operating are schematically illustrated in FIGS. 20 and 21, and an equivalent circuit diagram for explaining each part of the imaging element in Example 4 is illustrated in FIG. 6B. Also, a schematic disposition diagram of a first electrode, a transfer control electrode, and a charge accumulation electrode configuring a photoelectric conversion unit of the imaging element in Example 4 is illustrated in FIG. 22, and a schematic transparent perspective view of the first electrode, the transfer control electrode, the charge accumulation electrode, a second electrode, and a contact hole portion is illustrated in FIG. 23.

The imaging element and the stacked imaging element in Example 4 further include a transfer control electrode (charge transfer electrode) 25 that is disposed to be separated from a first electrode 21 and a charge accumulation electrode 24 and is disposed to face an inorganic oxide semiconductor material layer 23B via an insulating layer 82, between the first electrode 21 and the charge accumulation electrode 24. The transfer control electrode 25 is connected to a pixel drive circuit configuring a drive circuit via a connecting hole 68B, a pad portion 68A, and a wiring V_{OT} provided inside an interlayer insulating layer 81.

Hereinafter, operations of the imaging element (first imaging element 10) in Example 4 will be described with reference to FIGS. 20 and 21. Note that in FIGS. 20 and 21, values of a potential applied to the charge accumulation electrode 24 and a potential at the point P_{D} are different.

In a charge accumulation period, a potential V₁₁ is applied to the first electrode 21, a potential V₃₁ is applied to the charge accumulation electrode 24, and a potential V₅₁ is applied to the transfer control electrode 25, from the drive circuit. Photoelectric conversion occurs in the photoelectric conversion layer 23A due to light that is incident on the photoelectric conversion layer 23A. Holes generated through photoelectric conversion are fed from the second electrode 22 to the drive circuit via a wiring V_{OU}. On the other hand, since the potential of the first electrode 21 is set to be higher than the potential of the second electrode 22, that is, a positive potential is applied to the first electrode 21, and a negative potential is applied to the second electrode 22, for example, V₃₁>V₅₁ (for example, V₃₁>V₁₁>V₅₁ or V₁₁>V₃₁>V₅₁) is set. In this manner, electrons generated through photoelectric conversion are attracted by the charge accumulation electrode 24 and stop at the region of the inorganic oxide semiconductor material layer 23B and the like facing the charge accumulation electrode 24. In other words, charge is accumulated in the inorganic oxide semiconductor material layer 23B and the like. Since V₃₁>V₅₁, it is possible to reliably prevent electrons generated inside the photoelectric conversion layer 23A from moving toward the first electrode 21. With elapse of time of the photoelectric conversion, the potential in the region of the inorganic oxide semiconductor material layer 23B and the like facing the charge accumulation electrode 24 becomes a value that is yet closer to the negative side.

In a latter half of the charge accumulation period, a reset operation is performed. In this manner, a potential of a first floating diffusion layer FD₁ is reset, and a potential of the first floating diffusion layer FD₁ becomes a potential V_{DD} of a power source.

After the reset operation is completed, charge is read. In other words, a potential V₁₂ is applied to the first electrode 21, a potential V₃₂ is applied to the charge accumulation electrode 24, and a potential V₅₂ is applied to the transfer control electrode 25, from the drive circuit in the charge transfer period. Here, V₃₂≤V₅₂≤V₁₂ (preferably V₃₂<V₅₂<V₁₂) is set. In this manner, electrons stopping at the region of the inorganic oxide semiconductor material layer 23B and the like facing the charge accumulation electrode 24 are reliably read in the first electrode 21 and further the first floating diffusion layer FD₁. In other words, charge accumulated in the inorganic oxide semiconductor material layer 23B and the like is read by the control unit.

A series of operations including the charge accumulation, the reset operation, and the charge transfer are completed hitherto.

Operations of an amplification transistor TR1ₐₘₚ and a selection transistor TR1ₛₑₗ after the electrons are read in the first floating diffusion layer FD₁ are the same as operations of these transistors in the related art. Also, a series of operations including charge accumulation, reset operations, and charge transfer of the second imaging element 11 and the third imaging element 12 are similar to a series of operations including charge accumulation, a reset operation, and charge transfer in the related art.

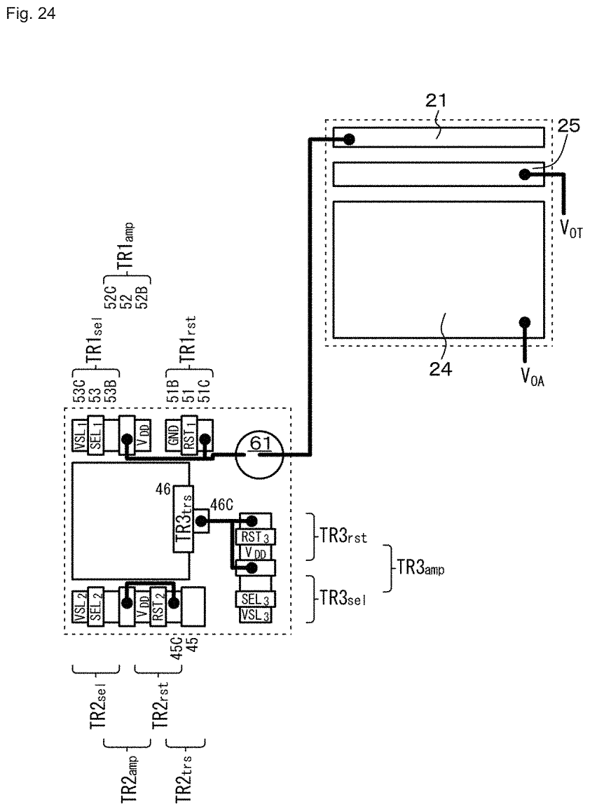

As illustrated in FIG. 24 as a schematic disposition diagram of the first electrode and the charge accumulation electrode configuring the modification example of the imaging element in Example 4 and the transistors configuring the control unit, the other source/drain region 51B of the reset transistor TR1ᵣₛₜ may be grounded instead of being connected to the power source V_{DD}.

### Example 5

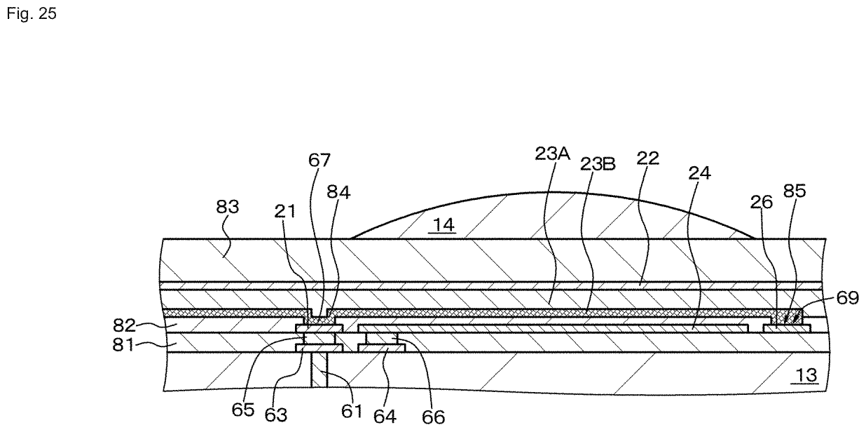

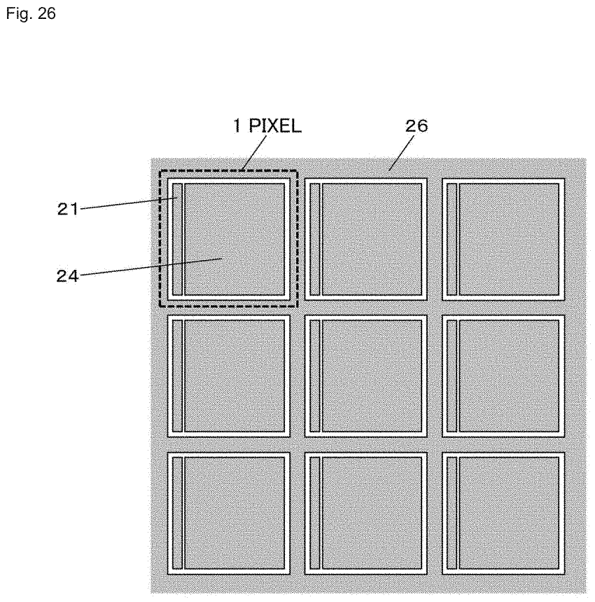

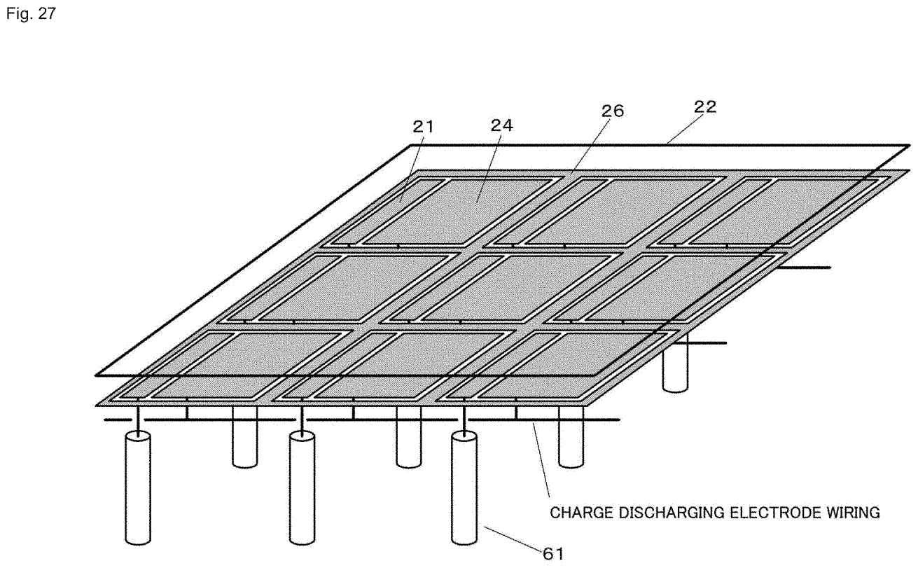

Example 5 is a modification of Examples 1 to 4 and relates to an imaging element or the like including the charge discharging electrode according to the present disclosure. A schematic partial sectional view of the imaging element in Example 5 is illustrated in FIG. 25, a schematic disposition diagram of a first electrode, a charge accumulation electrode, and a charge discharging electrode configuring a photoelectric conversion unit including the charge accumulation electrode of the imaging element in Example 5 is illustrated in FIG. 26, and a schematic transparent perspective view of the first electrode, the charge accumulation electrode, a charge discharging electrode, a second electrode, and a contact hole portion is illustrated in FIG. 27.

The imaging element in Example 5 further includes a charge discharging electrode 26 that is connected to an inorganic oxide semiconductor material layer 23B via a connecting portion 69 and is disposed to be separated from a first electrode 21 and a charge accumulation electrode 24. Here, the charge discharging electrode 26 is disposed such that it surrounds the first electrode 21 and the charge accumulation electrode 24 (that is, in a frame shape). The charge discharging electrode 26 is connected to a pixel drive circuit configuring a drive circuit. An inorganic oxide semiconductor material layer 23B extends inside the connecting portion 69. In other words, the inorganic oxide semiconductor material layer 23B extends inside a second opening portion 85 provided in the insulating layer 82, and the inorganic oxide semiconductor material layer 23B is connected to the charge discharging electrode 26. The charge discharging electrode 26 is shared by (commonly provided for) a plurality of imaging elements. An inclination spreading toward the upper side may be formed on a side surface of the second opening portion 85. The charge discharging electrode 26 can be used as floating diffusion or an overflow drain of the photoelectric conversion unit, for example.

In Example 5, a potential V₁₁ is applied to the first electrode 21 and a potential V₃₁ is applied to the charge accumulation electrode 24 and a potential V₆₁ is applied to a charge discharging electrode 26 from the drive circuit, and charge is accumulated in the inorganic oxide semiconductor material layer 23B or the like in the charge accumulation period. Photoelectric conversion occurs in the photoelectric conversion layer 23A due to light that is incident on the photoelectric conversion layer 23A. Holes generated through photoelectric conversion are fed from the second electrode 22 to the drive circuit via a wiring V_{OU}. On the other hand, since the potential of the first electrode 21 is set to be higher than the potential of the second electrode 22, that is, a positive potential is applied to the first electrode 21, and a negative potential is applied to the second electrode 22, V₆₁>V₁₁ (for example, V₃₁>V₆₁>V₁₁) is set. In this manner, electrons generated through photoelectric conversion are attracted by the charge accumulation electrode 24 and stop at the region of the inorganic oxide semiconductor material layer 23B and the like facing the charge accumulation electrode 24, and it is thus possible to reliably prevent the electrons from moving toward the first electrode 21. However, electrons that cannot be sufficiently attracted by the charge accumulation electrode 24 or cannot be accumulated in the inorganic oxide semiconductor material layer 23B and the like (so-called overflowed electrons) are fed to the drive circuit via the charge discharging electrode 26.

In the latter half of the charge accumulation period, a reset operation is performed. In this manner, the potential of the first floating diffusion layer FD₁ is reset, and the potential of the first floating diffusion layer FD₁ becomes a potential V_{DD} of a power source.

After the reset operation is completed, charge is read. In other words, a potential V₁₂ is applied to the first electrode 21 and a potential V₃₂ is applied to the charge accumulation electrode 24 and a potential V₆₂ is applied to the charge discharging electrode 26 from the drive circuit in the charge transfer period. Here, V₆₂<V₁₂ (for example, V₆₂<V₃₂<V₁₂) is set. In this manner, the electrons stopping at the region of the inorganic oxide semiconductor material layer 23B and the like facing the charge accumulation electrode 24 are reliably read in the first electrode 21 and further the first floating diffusion layer FD₁. In other words, the charge accumulated in the inorganic oxide semiconductor material layer 23B and the like is read by the control unit.

As described above, a series of operations including the charge accumulation, the reset operation, and the charge transfer are completed.

Operations of an amplification transistor TR1ₐₘₚ and a selection transistor TR1ₛₑₗ after electrons are read in the first floating diffusion layer FD₁ are the same as operations of these transistors in the related art. Also, a series of operations including the charge accumulation, the reset operation, and the charge transfer of the second imaging element and the third imaging element, for example, are similar to a series of operations including charge accumulation, a reset operation, and charge transfer in the related art.

In Example 5, since so-called overflowed electrons are fed to the drive circuit via the charge discharging electrode 26, it is possible to curb leaking to the charge accumulation units of adjacent pixels and to curb occurrence of blooming. It is thus possible to improve imaging performance of the imaging element.

### Example 6

Example 6 is a modification of Examples 1 to 5 and relates to an imaging element or the like including a plurality of charge accumulation electrode segments according to the present disclosure.

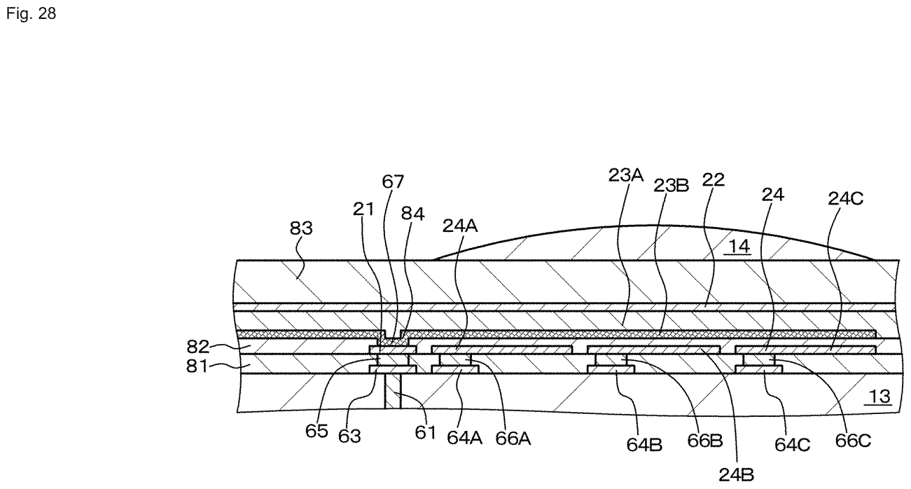

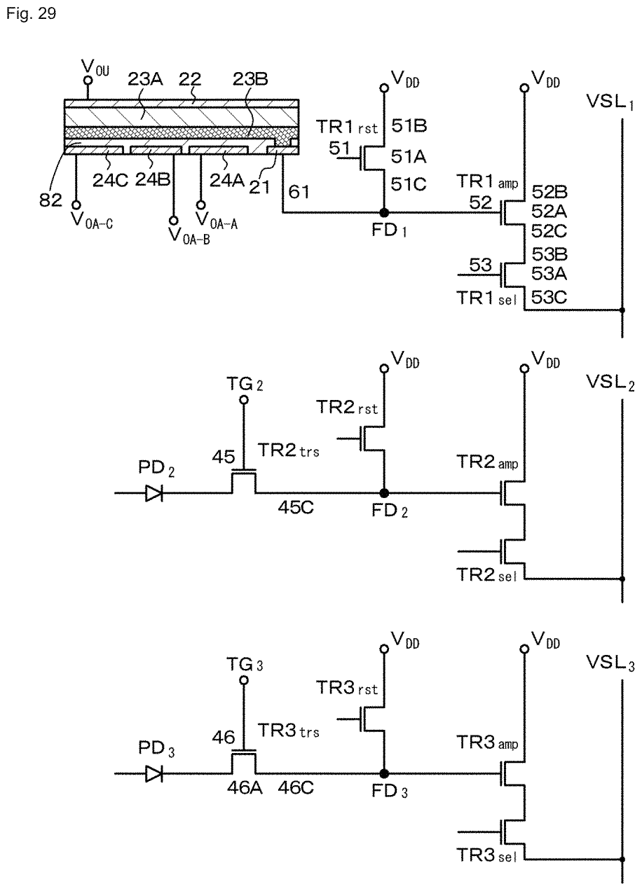

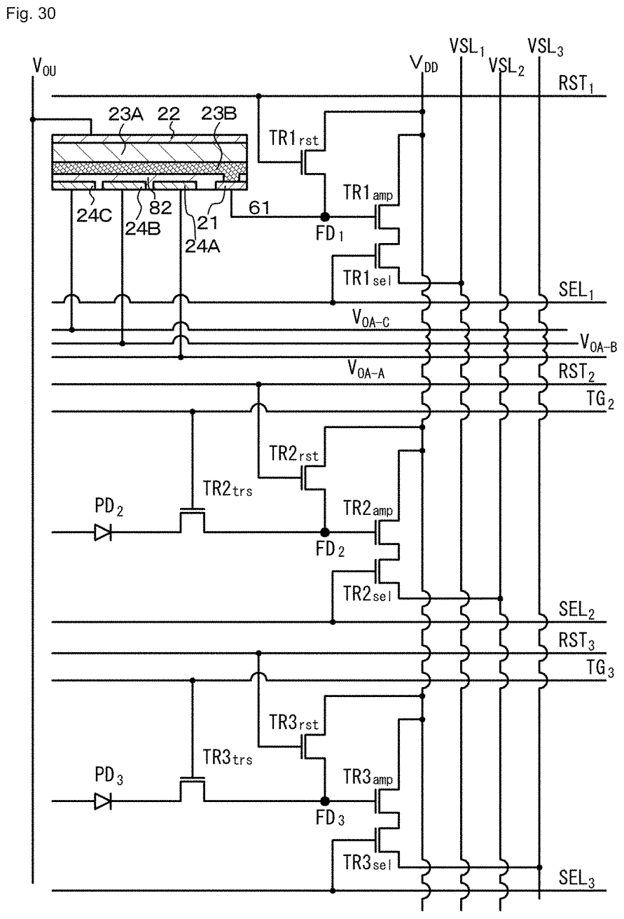

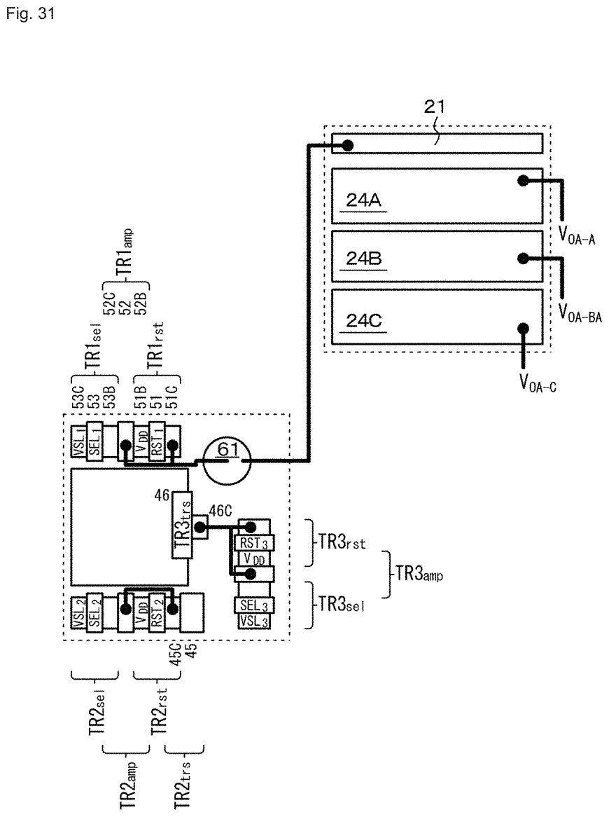

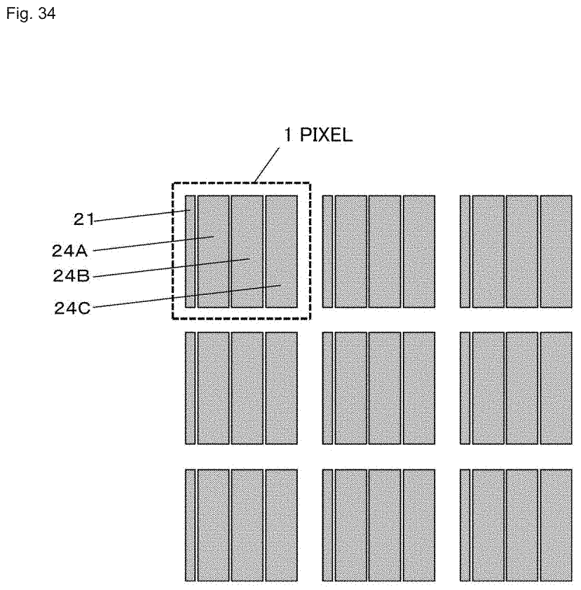

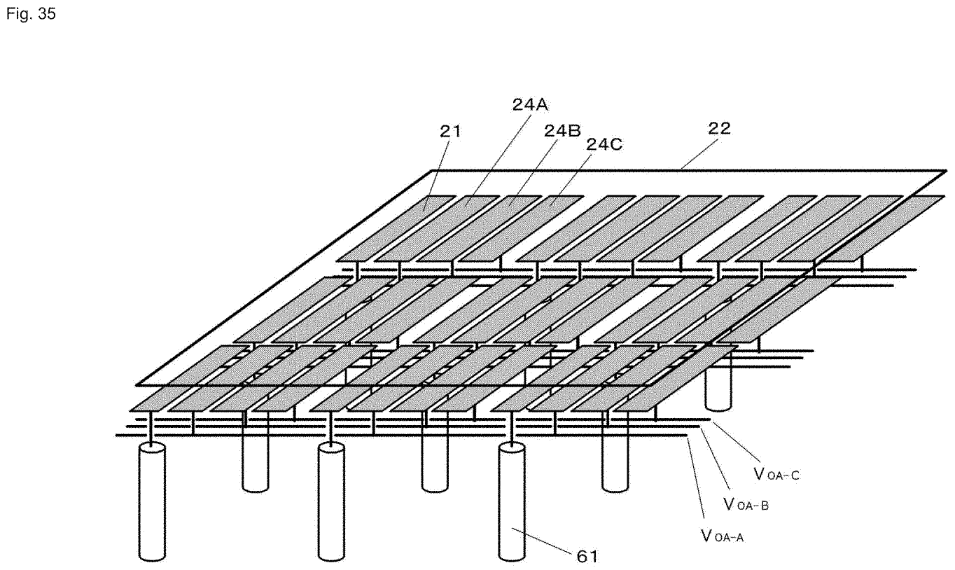

A schematic partial sectional view of a part of an imaging element in Example 6 is illustrated in FIG. 28, equivalent circuit diagrams of the imaging element in Example 6 are illustrated in FIGS. 29 and 30, a schematic disposition diagram of a first electrode and a charge accumulation electrode configuring a photoelectric conversion unit including the charge accumulation electrode of the imaging element in Example 6 and transistors configuring a control unit is illustrated in FIG. 31, states of a potential at each part when the imaging element in Example 6 is operating are schematically illustrated in FIGS. 32 and 33, and an equivalent circuit diagram for explaining each part of the imaging element in Example 6 is illustrated in FIG. 6C. Also, a schematic disposition diagram of the first electrode and the charge accumulation electrode configuring the photoelectric conversion unit including the charge accumulation electrode of the imaging element in Example 6 is illustrated in FIG. 34, and a schematic transparent perspective view of the first electrode, the charge accumulation electrode, a second electrode, and a contact hole portion is illustrated in FIG. 35.

In Example 6, the charge accumulation electrode 24 is configured by a plurality of charge accumulation electrode segments 24A, 24B, and 24C. The number of the charge accumulation electrode segments may be two or more and is three in Example 6. Also, the potential of the first electrode 21 is higher than the potential of the second electrode 22 in the imaging element in Example 6, that is, a positive potential is applied to the first electrode 21, and a negative potential is applied to the second electrode 22, for example. Additionally, a potential applied to the charge accumulation electrode segment 24A located at a position closest to the first electrode 21 is higher than a potential applied to the charge accumulation electrode segment 24C located at the position furthest from the first electrode 21 in the charge transfer period. By applying a potential gradient to the charge accumulation electrode 24 in this manner, electrons stopping at the region of the inorganic oxide semiconductor material layer 23B and the like facing the charge accumulation electrode 24 are further reliably read in the first electrode 21 and further a first floating diffusion layer FD₁. In other words, charge accumulated in the inorganic oxide semiconductor material layer 23B and the like is read by the control unit.

In the example illustrated in FIG. 32, electrons stopping at the region of the inorganic oxide semiconductor material layer 23B and the like are read at once in the first floating diffusion layer FD₁ by setting a potential of the charge accumulation electrode segment 24C<a potential of the charge accumulation electrode segment 24B<a potential of the charge accumulation electrode segment 24A in the charge transfer period. On the other hand, in the example illustrated in FIG. 33, electrons stopping at the region of the inorganic oxide semiconductor material layer 23B and the like facing the charge accumulation electrode segment 24C are moved to the region of the inorganic oxide semiconductor material layer 23B and the like facing the charge accumulation electrode segment 24B, electrons stopping at the region of the inorganic oxide semiconductor material layer 23B and the like facing the charge accumulation electrode segment 24B are then moved to the region of the inorganic oxide semiconductor material layer 23B and the like facing the charge accumulation electrode segment 24A, and electrons stopping at the region of the inorganic oxide semiconductor material layer 23B and the like facing the charge accumulation electrode segment 24A are then reliably read in the first floating diffusion layer FD₁, by gradually changing the potential of the charge accumulation electrode segment 24C, the potential of the charge accumulation electrode segment 24B, and the potential of the charge accumulation electrode segment 24A (or changing it in a stepwise manner or a slope manner) in the charge transfer period.

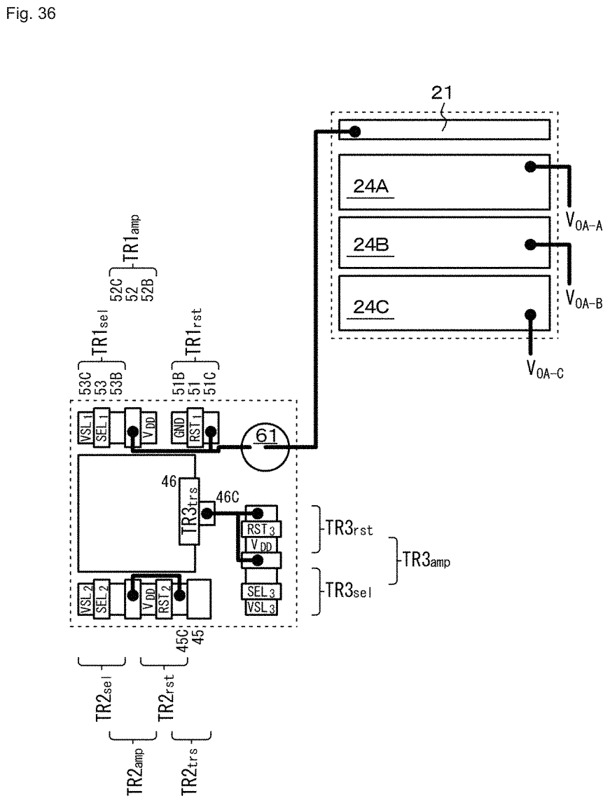

As illustrated in FIG. 36 as a schematic disposition diagram of the first electrode and the charge accumulation electrode configuring the modification example of the imaging element in Example 6 and the transistors configuring the control unit, the other source/drain region 51B of the reset transistor TR1ᵣₛₜ may be grounded instead of being connected to the power source V_{DD}.

### Example 7

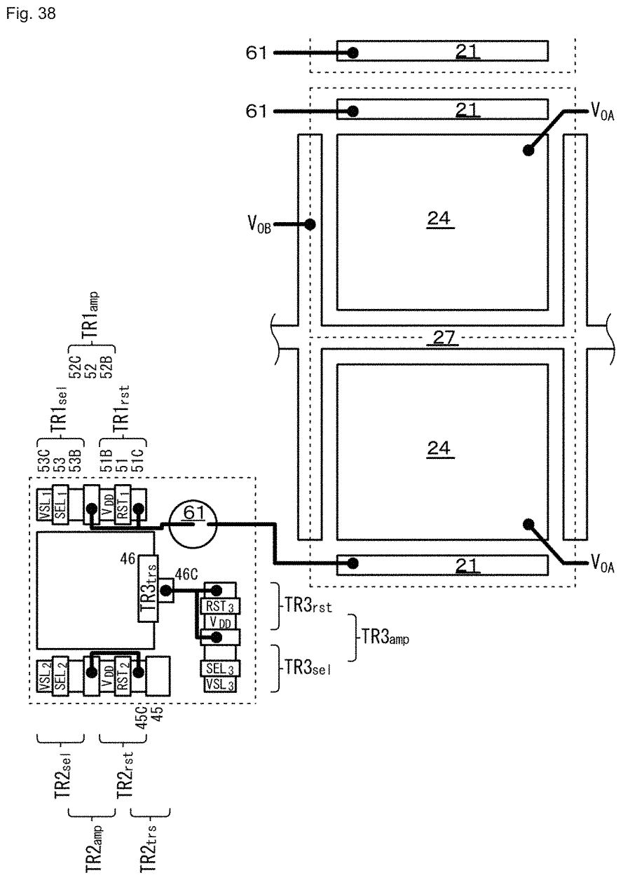

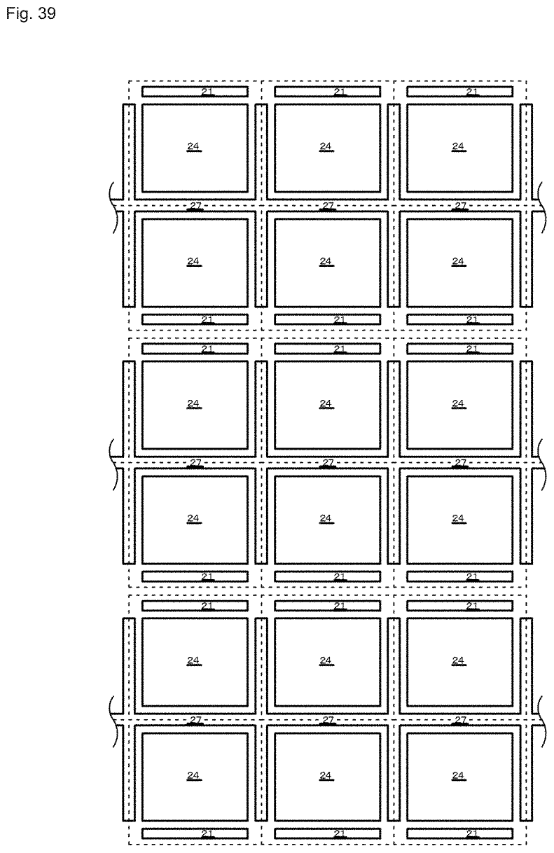

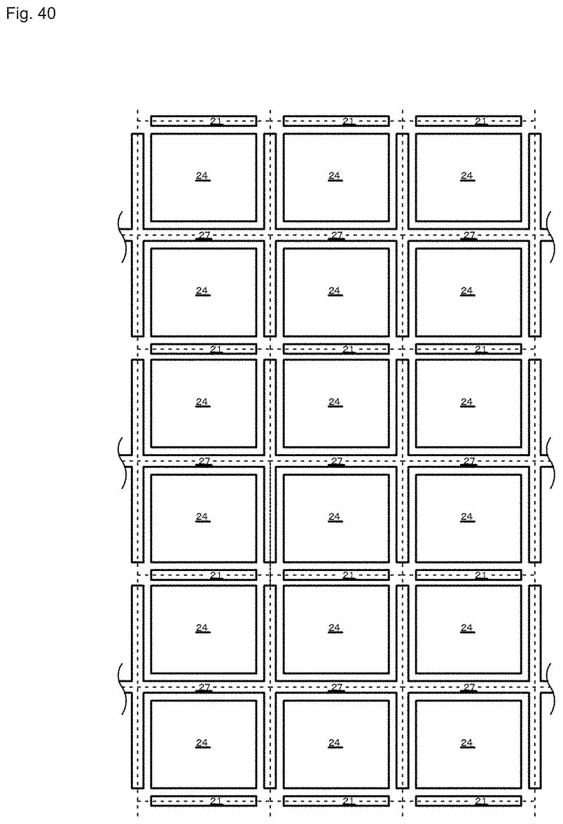

Example 7 is a modification of Examples 1 to 6 and relates to an imaging element or the like including the charge movement control electrode according to the present disclosure, specifically, an imaging element or the like including a lower charge movement control electrode (lower side charge movement control electrode) according to the present disclosure. A schematic partial sectional view of a part of the imaging element in Example 7 is illustrated in FIG. 37, a schematic disposition diagram of a first electrode, a charge accumulation electrode, and the like configuring the imaging element in Example 7 and transistors configuring a control unit is illustrated in FIG. 38, and schematic disposition diagrams of the first electrode, the charge accumulation electrode, and a lower charge movement control electrode configuring a photoelectric conversion unit including a charge accumulation electrode of the imaging element in Example 7 are illustrated in FIGS. 39 and 40.

In the imaging element in Example 7, a lower charge movement control electrode 27 is formed in a region that faces, via an insulating layer 82, a region (a region −A of the photoelectric conversion unit) 23A of a photoelectric conversion laminated body 23 located between adjacent imaging elements. In other words, a lower charge movement control electrode 27 is formed below a portion (a region −A of the insulating layer 82) 82_{A} of the insulating layer 82 in a region (region −a) sandwiched between a charge accumulation electrode 24 and a charge accumulation electrode 24 configuring each of adjacent imaging elements. The lower charge movement control electrode 27 is provided to be separated from the charge accumulation electrode 24. In other words, the lower charge movement control electrode 27 is provided such that it surrounds the charge accumulation electrode 24 and is separated from the charge accumulation electrode 24, and the lower charge movement control electrode 27 is disposed to face the region −A (23A) of the photoelectric conversion layer via the insulating layer 82. The lower charge movement control electrode 27 is shared by the imaging elements. Additionally, the lower charge movement control electrode 27 is also connected to the drive circuit. Specifically, the lower charge movement control electrode 27 is connected to a vertical drive circuit 112 configuring the drive circuit via a connection hole 27A, a pad portion 27B, and a wiring V_{OB} provided inside an interlayer insulating layer 81. The lower charge movement control electrode 27 may be formed in the same level as the first electrode 21 or the charge accumulation electrode 24 or may be formed in a different level (specifically, a level below the first electrode 21 or the charge accumulation electrode 24). In the former case, it is possible to shorten the distance between the charge movement control electrode 27 and the photoelectric conversion layer 23A, and it is thus easy to control a potential. On the other hand, in the latter case, it is possible to shorten the distance between the charge movement control electrode 27 and the charge accumulation electrode 24, which is advantageous for miniaturization.

In the imaging element in Example 7, since an absolute value of a potential applied to a portion of the photoelectric conversion layer 23A facing the charge accumulation electrode 24 is a value that is larger than an absolute value of a potential applied to the region −A of the photoelectric conversion layer 23A when light is incident on the photoelectric conversion layer 23A and photoelectric conversion occurs in the photoelectric conversion layer 23A, charge generated through photoelectric conversion is strongly attracted by a portion of the inorganic oxide semiconductor material layer 23B facing the charge accumulation electrode 24. As a result, it is possible to curb flowing of the charge generated through the photoelectric conversion into adjacent imaging elements, and degradation of quality does not occur in imaged videos (images). Further, since the lower charge movement control electrode 27 is formed in a region facing the region −A of the photoelectric conversion layer 23A via the insulating layer, it is possible to control an electric field and a potential of the region −A of the photoelectric conversion layer 23A located above the lower charge movement control electrode 27. As a result, since it is possible to curb flowing of the charge generated through photoelectric conversion into adjacent imaging elements by the lower charge movement control electrode 27, degradation of quality does not occur in imaged videos (images).

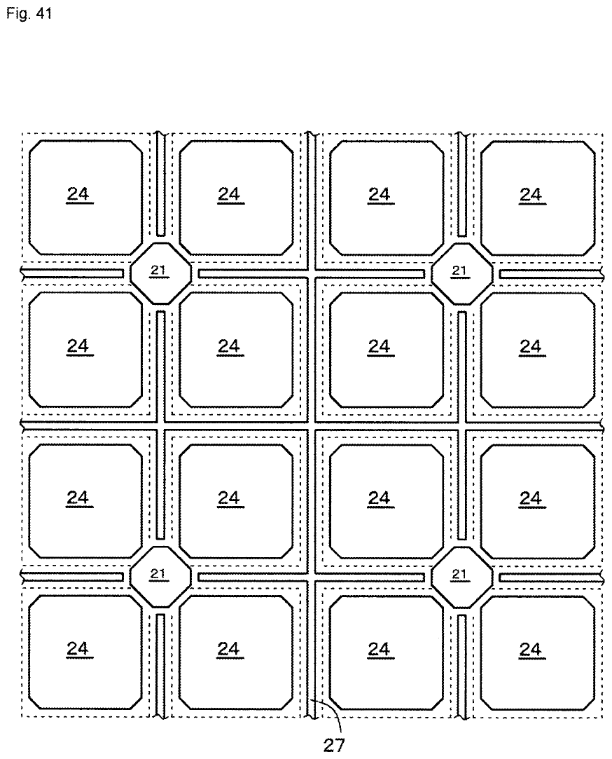

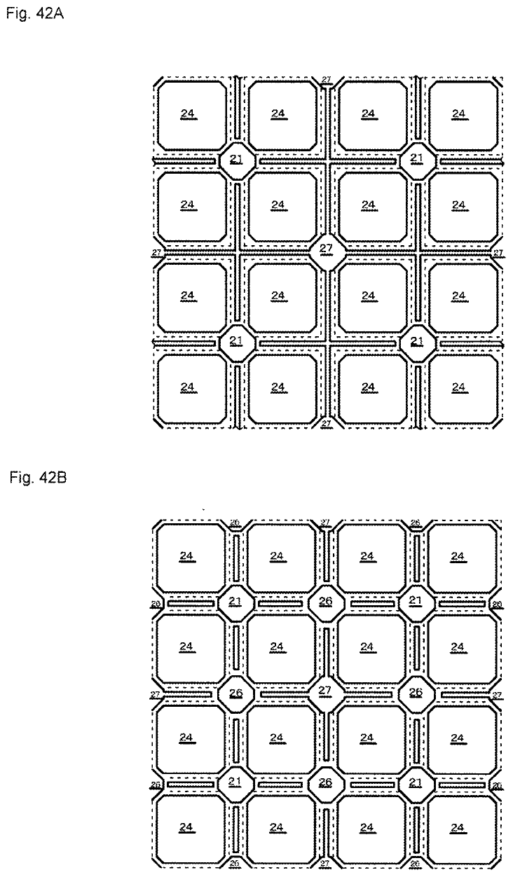

In the example illustrated in FIGS. 39 and 40, the lower charge movement control electrode 27 is formed below a portion 82_{A} of the insulating layer 82 in the region (region −a) sandwiched between the charge accumulation electrode 24 and the charge accumulation electrode 24. On the other hand, in the example illustrated in FIGS. 41, 42A, and 42B, the lower charge movement control electrode 27 is formed below a portion of the insulating layer 82 in a region surrounded by four charge accumulation electrodes 24. Note that the examples illustrated in FIGS. 41, 42A, and 42B are also solid-state imaging devices with a first configuration and a second configuration. Also, one common first electrode 21 is provided to correspond to four charge accumulation electrodes 24 in four imaging elements.

In the example illustrated in FIG. 42B, one common first electrode 21 is provided to correspond to four charge accumulation electrodes 24 in four imaging elements, the lower charge movement control electrode 27 is formed below a portion of the insulating layer 82 in the region surrounded by the four charge accumulation electrodes 24, and further, a charge discharging electrode 26 is formed below a portion of the insulating layer 82 in the region surrounded by the four charge accumulation electrodes 24. As described above, the charge discharging electrode 26 can be used, for example, as floating diffusion or an overflow drain of the photoelectric conversion unit.

### Example 8

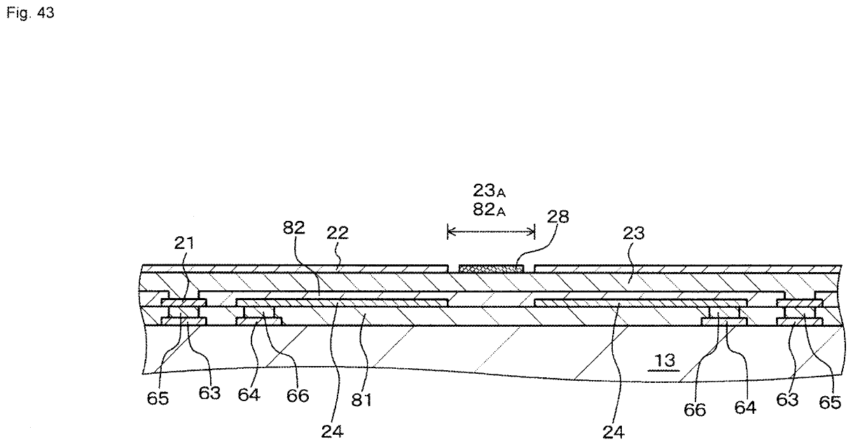

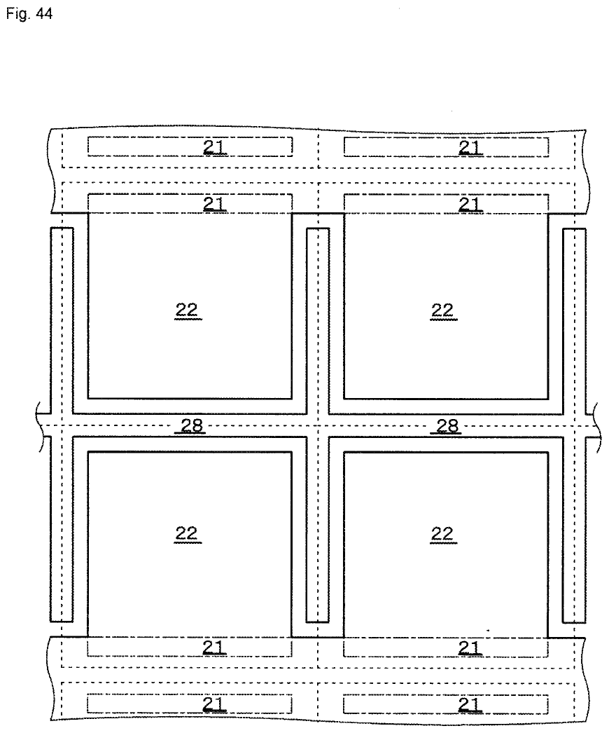

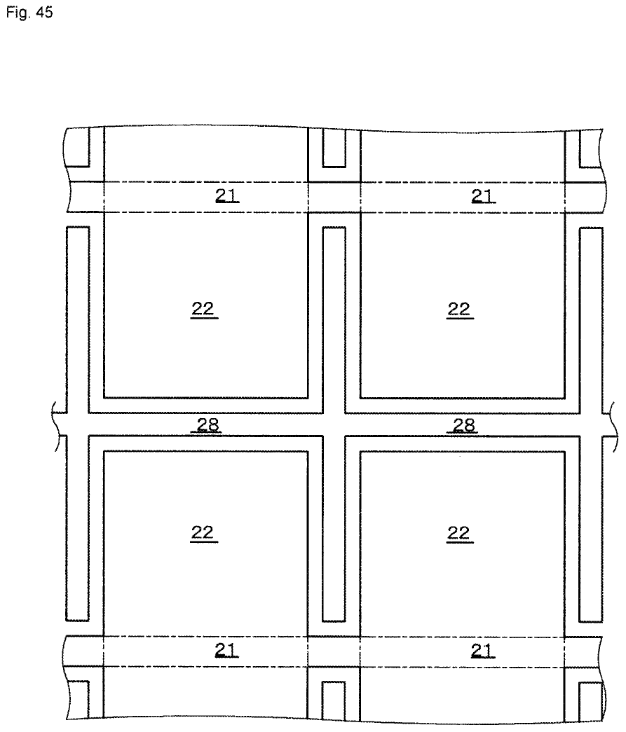

Example 8 is a modification of Example 7 and relates to an imaging element or the like including the upper charge movement control electrode (upper-side charge movement control electrode) according to the present disclosure. A schematic sectional view of a part of imaging elements (two imaging elements aligned side by side) in Example 8 is illustrated in FIG. 43, and schematic plan views of a part of the imaging elements (2×2 imaging elements aligned side by side) in Example 8 are illustrated in FIGS. 44 and 45. In the imaging elements in Example 8, an upper charge movement control electrode 28 is formed instead of the formation of a second electrode 22 above a region 23A of a photoelectric conversion laminated body 23 located between the adjacent imaging elements. The upper charge movement control electrode 28 is provided to be separated from the second electrode 22. In other words, the second electrode 22 is provided for each imaging element, and the upper charge movement control electrode 28 is provided above a region-A of the photoelectric conversion laminated body 23 such that it surrounds at least a part of the second electrode 22 and is separated from the second electrode 22. The upper charge movement control electrode 28 is formed in the same level as the second electrode 22.

Note that in the example illustrated in FIG. 44, one charge accumulation electrode 24 is provided to correspond to one first electrode 21 in one imaging element. On the other hand, in the modification example illustrated in FIG. 45, one common first electrode 21 is provided to correspond to two charge accumulation electrodes 24 in two imaging elements. A schematic sectional view of the imaging elements (the two imaging elements aligned side by side) in Example 8 illustrated in FIG. 43 corresponds to FIG. 45.

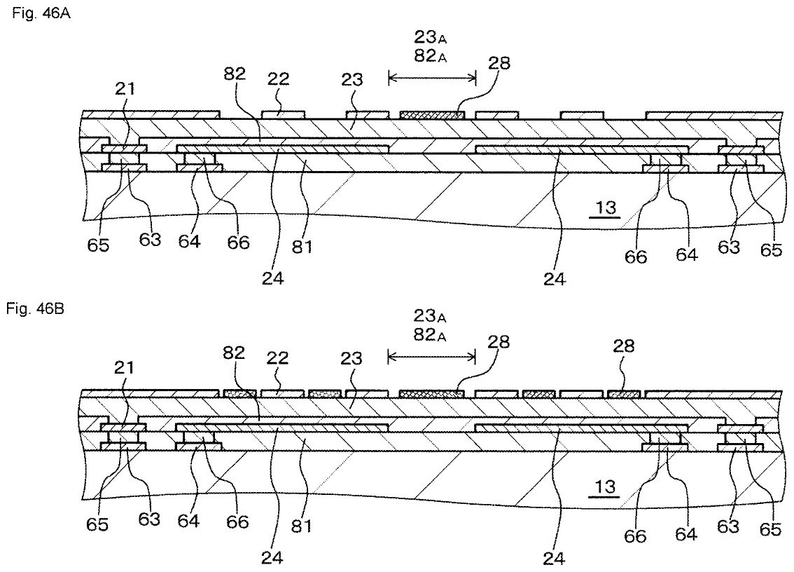

Also, as illustrated in FIG. 46A as a schematic sectional view of a part of the imaging elements (the two imaging elements aligned side by side) in Example 8, a plurality of second electrodes 22 may be provided in a split manner, and different potentials may be individually applied to each of the split second electrodes 22. Moreover, the upper charge movement control electrode 28 may be provided between the split second electrodes 22 as illustrated in FIG. 46B.

In Example 8, the second electrode 22 located on the light incident side is shared by the imaging elements aligned in the left-right direction in the plane of paper in FIG. 44 and is shared by a pair of imaging elements aligned in the up-down direction in the plane of paper in FIG. 44. Also, the upper charge movement control electrode 28 is also shared by the imaging elements aligned in the left-right direction in the plane of paper in FIG. 44 and is shared by a pair of imaging elements aligned in the up-down direction in the plane of paper in FIG. 44. The second electrode 22 and the upper charge movement control electrode 28 can be obtained by forming a material layer configuring the second electrode 22 and the upper charge movement control electrode 28 above the photoelectric conversion laminated body 23 and then patterning the material layer. Each of the second electrode 22 and the upper charge movement control electrode 28 is connected to separate wirings (not illustrated), and these wirings are connected to the drive circuit. The wiring connected to the second electrode 22 is shared by a plurality of imaging elements. The wiring connected to the upper charge movement control electrode 28 is also shared by a plurality of imaging elements.

In the imaging elements in Example 8, a potential V₂₁ is applied to the second electrode 22 and a potential V₄₁ is applied to the upper charge movement control electrode 28 from the drive circuit, and charge is accumulated in the photoelectric conversion laminated body 23 in a charge accumulation period, a potential V₂₂ is applied to the second electrode 22 and a potential V₄₂ is applied to the upper charge movement control electrode 28 from the drive circuit, and the charge accumulated in the photoelectric conversion laminated body 23 is read by the control unit via the first electrode 21 in a charge transfer period. Here, since the potential of the first electrode 21 is assumed to be higher than the potential of the second electrode 22, V₂₁>V₄₁ and V₂₂>V₄₂ are satisfied.

As described above, since the charge movement control electrode is formed instead of formation of the second electrode above the region of the photoelectric conversion layer located between adjacent imaging elements in the imaging elements in Example 8, it is possible to curb, by the charge movement control electrode, flowing of the charge generated through photoelectric conversion into adjacent imaging elements, and degradation of quality of captured videos (images) does not occur.

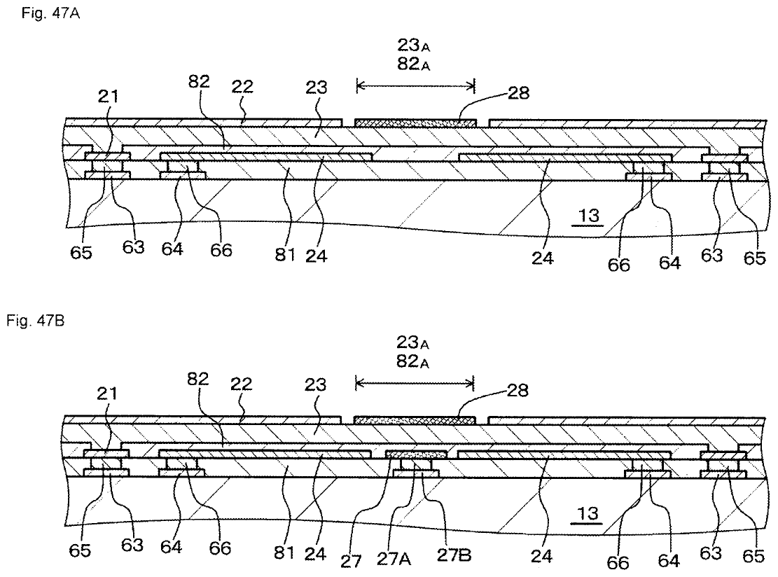

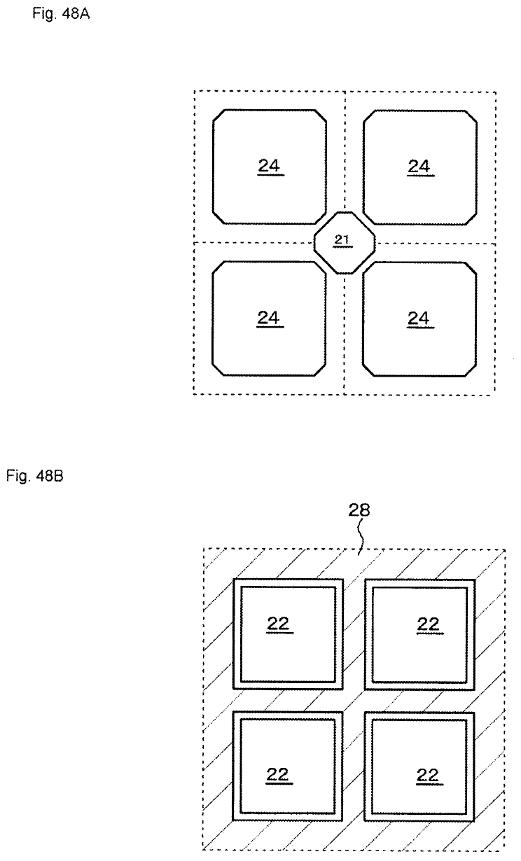

A schematic sectional view of a part of a modification example of the imaging elements (the two imaging elements aligned side by side) in Example 8 is illustrated in FIG. 47A, and schematic plan views of a part are illustrated in FIGS. 48A and 48B. In this modification example, the second electrode 22 is provided for each imaging element, the upper charge movement control electrode 28 is provided such that it surrounds at least a part of the second electrode 22 and is separated from the second electrode 22, and a part of the charge accumulation electrode 24 is present below the upper charge movement control electrode 28. The second electrode 22 is provided with a size that is smaller than that of the charge accumulation electrode 24 above the charge accumulation electrode 24.

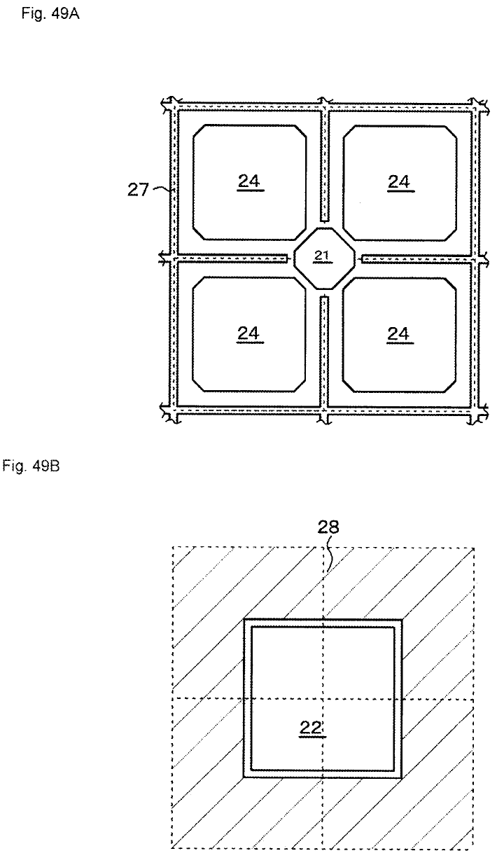

A schematic sectional view of a part of a modification example of the imaging elements (the two imaging elements aligned side by side) in Example 8 is illustrated in FIG. 47B, and schematic plan views of a part are illustrated in FIGS. 49A and 49B. In the modification example, the second electrode 22 is provided for each imaging element, the upper charge movement control electrode 28 is provided such that it surrounds at least a part of the second electrode 22 and is separated from the second electrode 22, a part of the charge accumulation electrode 24 is present below the upper charge movement control electrode 28, and further, a lower charge movement control electrode (lower side charge movement control electrode) 27 is provided below the upper charge movement control electrode (upper side charge movement control electrode) 28. The size of the second electrode 22 is smaller than that in the modification example illustrated in FIG. 47A. In other words, a region of the second electrode 22 facing the upper charge movement control electrode 28 is located on the side closer to the first electrode 21 than a region of the second electrode 22 facing the upper charge movement control electrode 28 in the modification example illustrated in FIG. 47A. The charge accumulation electrode 24 is surrounded by the lower charge movement control electrode 27.

### Example 9

Example 9 relates to a solid-state imaging device with first and second configurations.

The solid-state imaging device in Example 9 includes a plurality of imaging elements, each of which includes a photoelectric conversion unit that is configured of a first electrode 21, an inorganic oxide semiconductor material layer 23B, a photoelectric conversion layer 23A, and a second electrode 22 being laminated, the photoelectric conversion unit further including a charge accumulation electrode 24 that is disposed to be separated from the first electrode 21 and is disposed to face the inorganic oxide semiconductor material layer 23B via an insulating layer 82, the plurality of imaging elements configure an imaging element block, and the first electrode 21 is shared by the plurality of imaging elements configuring the imaging element block.

Alternatively, the solid-state imaging device in Example 9 includes a plurality of imaging elements described in Examples 1 to 8.

In Example 9, one floating diffusion layer is provided for the plurality of imaging elements. The plurality of imaging elements can share the one floating diffusion layer by appropriately controlling a timing of a charge transfer period. In this case, the plurality of imaging elements can share one contact hole portion.

Note that the solid-state imaging device in Example 9 has a configuration and a structure that are substantially similar to those of the solid-state imaging devices described in Examples 1 to 8 other than that the first electrode 21 is shared by the plurality of imaging elements configuring the imaging element block.

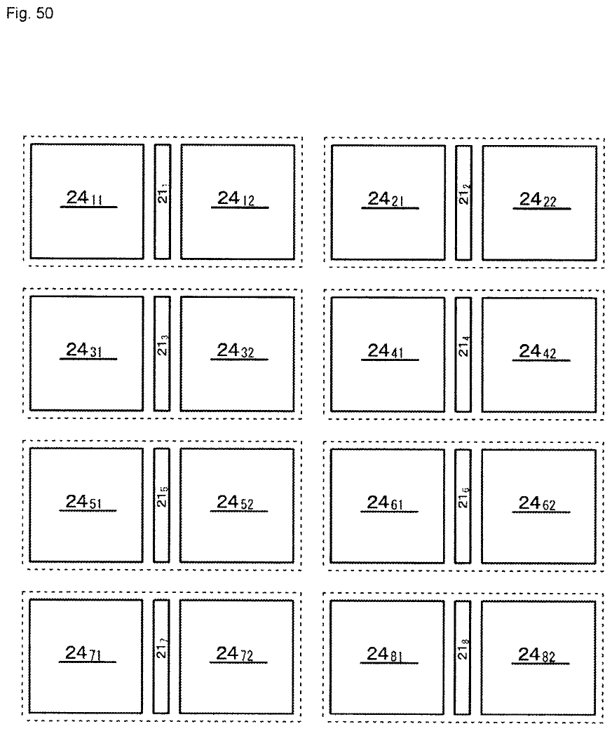

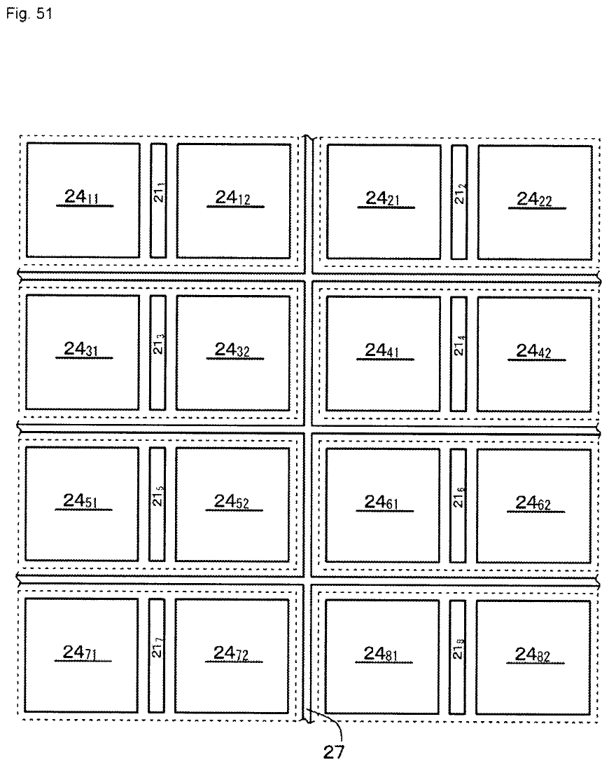

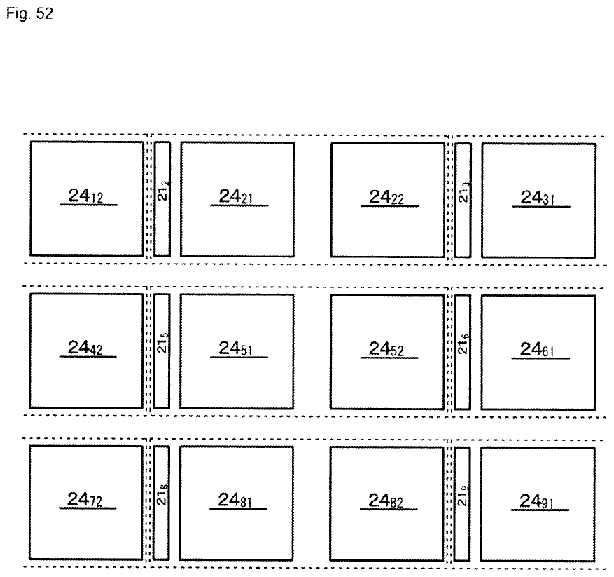

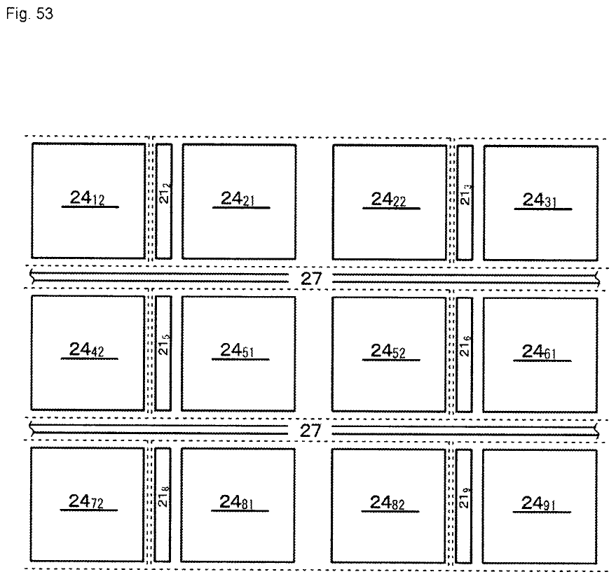

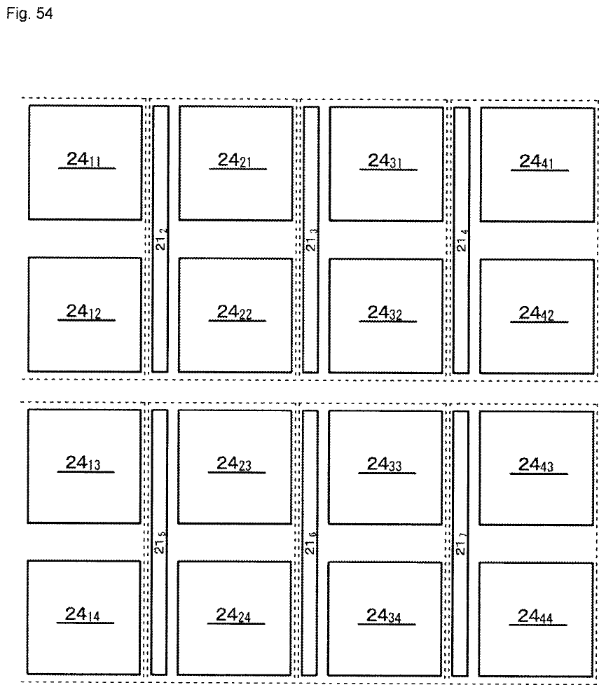

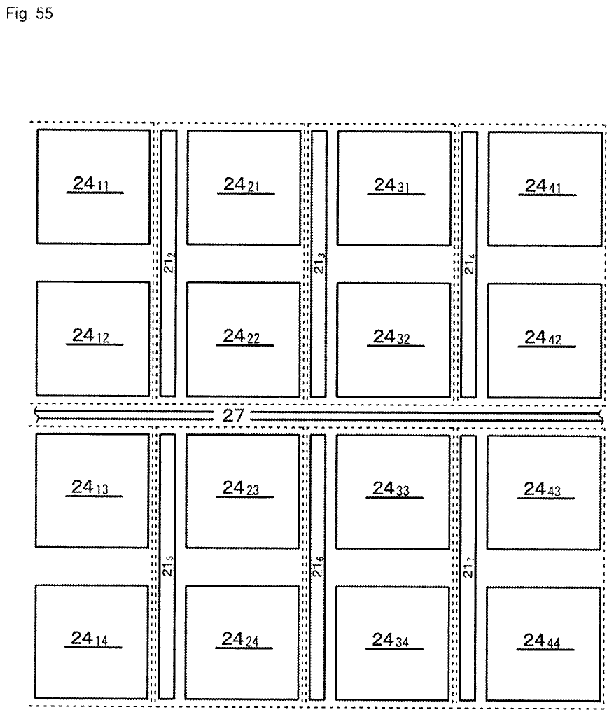

Disposition states of the first electrode 21 and the charge accumulation electrode 24 in the solid-state imaging device in Example 9 are schematically illustrated in FIG. 50 (Example 9), FIG. 51 (first modification example of Example 9), FIG. 52 (second modification example of Example 9), FIG. 53 (third modification example of Example 9), and FIG. 54 (fourth modification example of Example 9). FIGS. 50, 51, 54, and 55 illustrate sixteen imaging elements, and FIGS. 52 and 53 illustrate twelve imaging elements. Also, an imaging element block is configured of two imaging elements. The imaging element blocks are illustrated by being surrounded by dotted lines. The subscripts applied to the first electrodes 21 and the charge accumulation electrodes 24 are for distinguishing the first electrodes 21 and the charge accumulation electrodes 24. The same applies to the following description. Also, one on-chip micro lens (not illustrated in FIGS. 50 to 57) is disposed above one imaging element. In addition, two charge accumulation electrodes 24 are disposed in one imaging element block with the first electrode 21 sandwiched therebetween (see FIGS. 50 and 51). Alternatively, one first electrode 21 is disposed to face two charge accumulation electrodes 24 aligned side by side (see FIGS. 54 and 55). In other words, the first electrode is disposed to be adjacent to the charge accumulation electrode in each imaging element. Alternatively, the first electrodes are disposed to be adjacent to the charge accumulation electrodes of a part of the plurality of imaging elements and are disposed not to be adjacent to the charge accumulation electrodes of remaining ones of the plurality of imaging elements (see FIGS. 52 and 53), and in this case, movement of charge from the remaining ones in the plurality of imaging elements to the first electrodes is movement via the part of the plurality of imaging elements. A distance A between a charge accumulation electrode configuring an imaging element and a charge accumulation electrode configuring an imaging element is preferably longer than a distance B between a first electrode and a charge accumulation electrode in an imaging element that is adjacent to the first electrode in order to reliably move charge from each imaging element to the first electrode. Also, an imaging element located at a further position from a first electrode preferably has a larger value of the distance A. In the example illustrated in FIGS. 51, 53, and 55, a charge movement control electrode 27 is disposed between the plurality of imaging elements configuring imaging element blocks. It is possible to reliably curb movement of the charge in the imaging element blocks located with the charge movement control electrode 27 sandwiched therebetween by disposing the charge movement control electrode 27. Note that when a potential applied to the charge movement control electrode 27 is defined as V₁₇, V₃₁>V₁₇ may be satisfied.

The charge movement control electrode 27 may be formed in the same level as the first electrode 21 or the charge accumulation electrode 24 on the side of the first electrode or may be formed in a different level (specifically, a level below the first electrode 21 or the charge accumulation electrode 24). In the former case, it is possible to shorten the distance between the charge movement control electrode 27 and the photoelectric conversion layer, and it is thus easy to control the potential. On the other hand, in the latter case, it is possible to shorten the distance between the charge movement control electrode 27 and the charge accumulation electrode 24, which is advantageous for microfabrication.

Hereinafter, operations of an imaging element block configured of a first electrode 21₂ and two charge accumulation electrodes 24₂₁ and 24₂₂ will be described.

In a charge accumulation period, a potential V₁₁ is applied to the first electrode 21₂, and a potential V₃₁ is applied to the charge accumulation electrodes 24₂₁ and 24₂₂ from a drive circuit. Photoelectric conversion occurs in a photoelectric conversion layer 23A due to light that is incident on the photoelectric conversion layer 23A. A hole generated through the photoelectric conversion is fed from a second electrode 22 to the drive circuit via a wiring V_{OU}. On the other hand, since the potential V₁₁ of the first electrode 21₂ is set to be higher than the potential V₂₁ of the second electrode 22, that is, since a positive potential is applied to the first electrode 21₂ and a negative potential is applied to the second electrode 22, for example, V₃₁>V₁₁ or preferably V₃₁>V₁₁ is set. In this manner, electrons generated through the photoelectric conversion are attracted by the charge accumulation electrodes 24₂₁ and 24₂₂ and stops at a region of an inorganic oxide semiconductor material layer 23B and the like that face the charge accumulation electrodes 24₂₁ and 24₂₂. In other words, the charge is accumulated in the inorganic oxide semiconductor material layer 23B and the like. Since V₃₁>V₁₁, the electrons generated inside the photoelectric conversion layer 23A do not move toward the first electrode 21₂. With elapse of time of the photoelectric conversion, the potential in the region of the inorganic oxide semiconductor material layer 23B and the like facing the charge accumulation electrodes 24₂₁ and 24₂₂ becomes a value that is yet closer to the negative side.

In the latter half of the charge accumulation period, a reset operation is performed. In this manner, the potential of the first floating diffusion layer is reset, and the potential of the first floating diffusion layer becomes a potential V_{DD} of a power source.

After the reset operation is completed, charge is read. In other words, in a charge transfer period, a potential V₂₁ is applied to the first electrode 21₂, a potential V_{32-A} is applied to the charge accumulation electrode 24₂₁, and a potential V_{32-B} is applied to the charge accumulation electrode 24₂₂ from the drive circuit. Here, V_{32-A}<V₂₁<V_{32-B} is set. Electrons stopping at the region of the inorganic oxide semiconductor material layer 23B and the like facing the charge accumulation electrode 24₂₁ are thus read in the first electrode 21₂ and further in the first floating diffusion layer. In other words, the charge accumulated in the region of the inorganic oxide semiconductor material layer 23B and the like facing the charge accumulation electrode 24₂₁ is read by a control unit. If the reading is completed, V_{32-B}<V_{32-A}<V₂₁ is set. Note that in the example illustrated in FIGS. 54 and 55, V_{32-B}<V₂₁<V_{32-A} may be set. Electrons stopping at the region of the inorganic oxide semiconductor material layer 23B and the like facing the charge accumulation electrode 24₂₂ are thus read in the first electrode 21₂ and further in the first floating diffusion layer. Also, in the example illustrated in FIGS. 52 and 53, electrons stopping at the region of the inorganic oxide semiconductor material layer 23B and the like facing the charge accumulation electrode 24₂₂ may be read in the first floating diffusion layer via a first electrode 21₃ to which the charge accumulation electrode 24₂₂ is adjacent. In this manner, the charge accumulated in the region of the inorganic oxide semiconductor material layer 23B and the like facing the charge accumulation electrode 24₂₂ is read in the control unit. Note that the potential of the first floating diffusion layer may be reset if reading of the charge accumulated in the region of the inorganic oxide semiconductor material layer 23B and the like facing the charge accumulation electrode 24₂₁ in the control unit is completed.

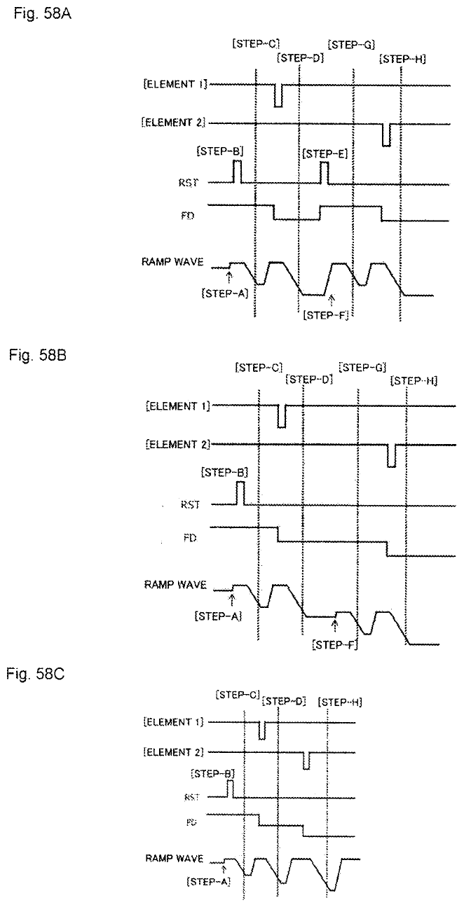

FIG. 58A illustrates a reading and driving example in the imaging element block in Example 9, and signals are read from two imaging elements corresponding to the charge accumulation electrode 24₂₁ and the charge accumulation electrode 24₂₂ in a flow including:

[Step-A] inputting an automatic zero signal to a comparator;
[Step-B] performing an operation of resetting one shared floating diffusion layer;
[Step-C] performing P-phase reading in the imaging element corresponding to the charge accumulation electrode 24₂₁ and moving charge to the first electrode 21₂;
[Step-D] performing D-phase reading in the imaging element corresponding to the charge accumulation electrode 24₂₁ and moving charge to the first electrode 21₂;
[Step-E] performing an operation of resetting the one shared floating diffusion layer;
[Step-F] inputting an automatic zero signal to the comparator;
[Step-G] performing P-phase reading in the imaging element corresponding to the charge accumulation electrode 24₂₂ and moving charge to the first electrode 21₂;
[Step-H] performing D-phase reading in the imaging element corresponding to the charge accumulation electrode 24₂₂ and moving charge to the first electrode 21₂.

On the basis of a correlated double sampling (CDS), a difference between the P-phase reading in [Step-C] and the D-phase reading in [Step-D] is a signal from the imaging element corresponding to the charge accumulation electrode 24₂₁, and a difference between the P-phase reading in [Step-G] and the D-phase reading in [Step-H] is a signal from the imaging element corresponding to the charge accumulation electrode 24₂₂.

Note that the operation in [Step-E] may be omitted (see FIG. 58B). Also, the operation in [Step-F] may be omitted, and in this case, [Step-G] may be further omitted (see FIG. 58C), the difference between the P-phase reading in [Step-C] and the D-phase reading in [Step-D] is a signal from the imaging element corresponding to the charge accumulation electrode 24₂₁, and the difference between the D-phase reading in [Step-D] and the D-phase reading in [Step-H] is a signal from the imaging element corresponding to the charge accumulation electrode 24₂₂.

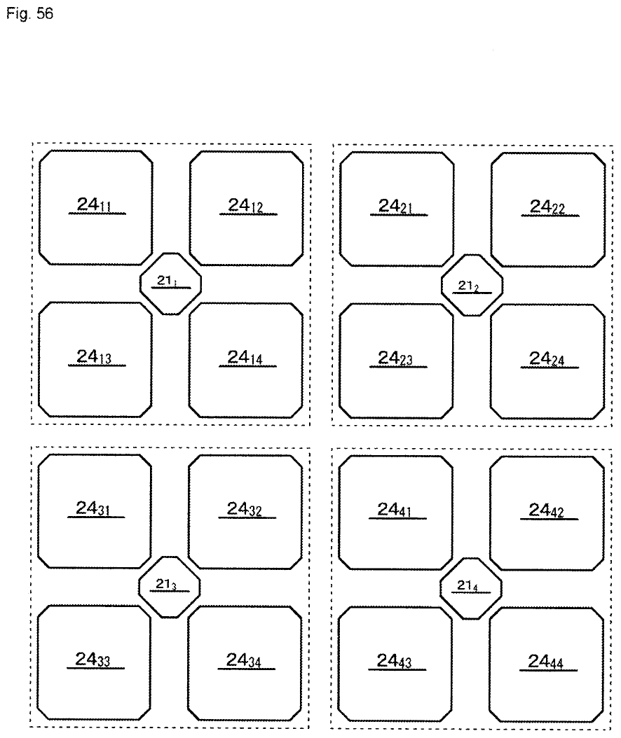

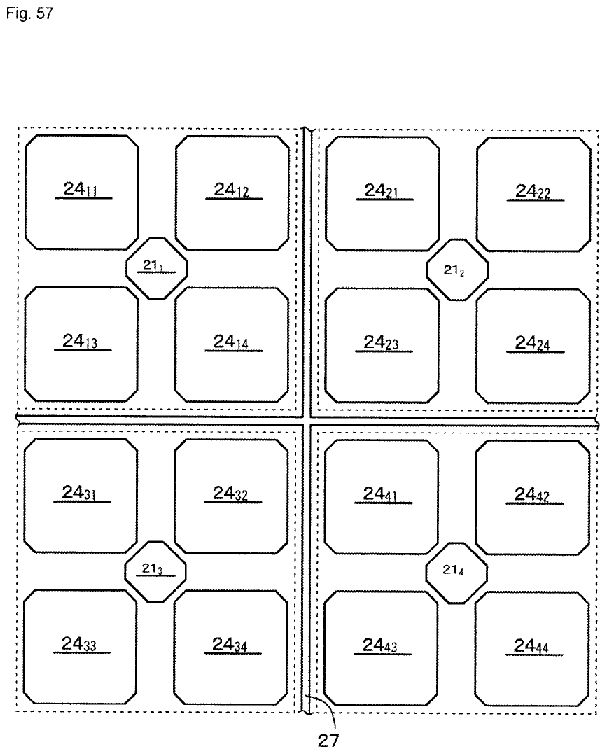

In modification examples with the first electrode 21 and the charge accumulation electrode 24 disposed in states schematically illustrated in FIG. 56 (sixth modification example of Example 9) and FIG. 57 (seventh modification example of Example 9), an imaging element block is configured of four imaging elements. Operations of these solid-state imaging devices can be similar to the operations of the solid-state imaging device illustrated in FIGS. 50 to 55.

Since the first electrode is shared by the plurality of imaging elements configuring the imaging element block in the solid-state imaging device in Example 9, it is possible to simplify and miniaturize the configuration and the structure in the pixel region in which a plurality of imaging elements are aligned. Note that the plurality of imaging elements provided for one floating diffusion layer may be configured of a plurality of imaging elements of a first type or may be configured of at least one imaging element of a first type and one or two or more imaging elements of a second type.

### Example 10

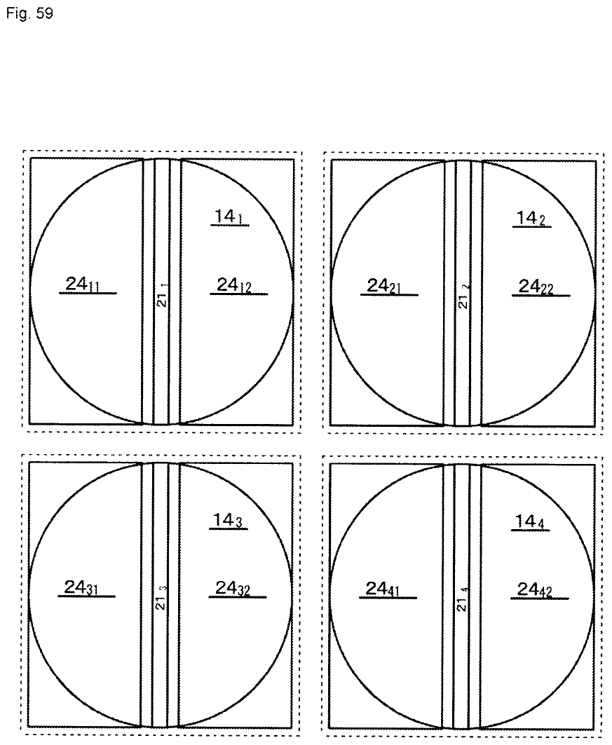

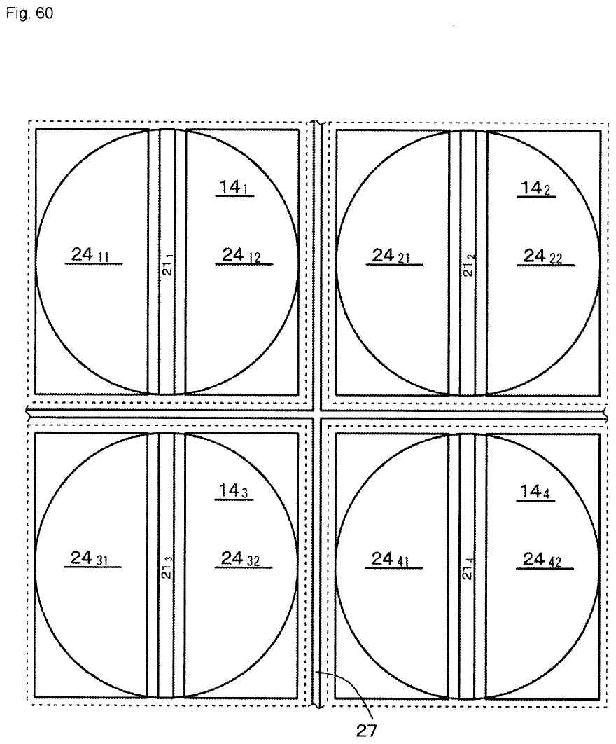

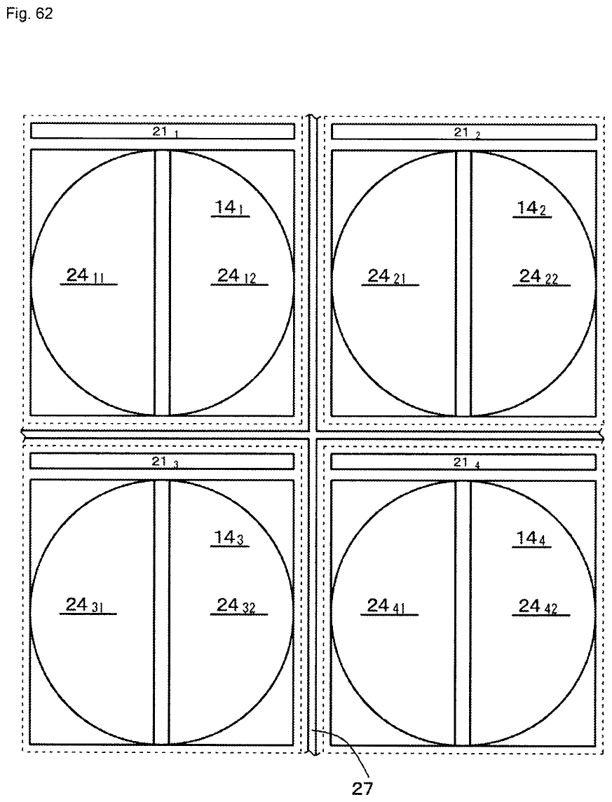

Example 10 is a modification of Example 9. In a solid-state imaging device in Example 10 with a first electrode 21 and a charge accumulation electrode 24 disposed in a state schematically illustrated in FIGS. 59, 60, 61, and 62, two imaging elements configure an imaging element block. Also, one on-chip micro lens 14 is disposed above the imaging element block. Note that in the example illustrated in FIGS. 60 and 62, a charge movement control electrode 27 is disposed between the plurality of imaging elements configuring the imaging element block.

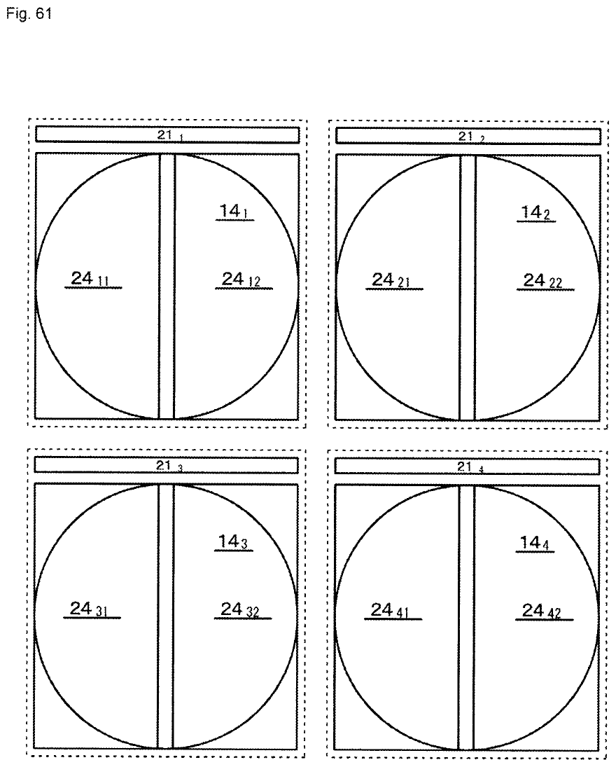

For example, photoelectric conversion layers corresponding to charge accumulation electrodes 24₁₁, 24₂₁, 24₃₁, and 24₄₁ configuring an imaging element block have high sensitivity to incident light from the obliquely right upper side in the drawing. Also, photoelectric conversion layers corresponding to charge accumulation electrodes 24₁₂, 24₂₂, 24₃₂, and 24₄₂ configuring an imaging element block have high sensitivity to incident light from the obliquely left upper side in the drawing. Therefore, it is possible to acquire image plane phase difference signals by combining the imaging element including the charge accumulation electrode 24₁₁ and the imaging element including the charge accumulation electrode 24₁₂. If a signal from the imaging element including the charge accumulation electrode 24₁₁ and a signal from the imaging element including the charge accumulation electrode 24₁₂ are added, then it is possible to configure one imaging element by a combination with these imaging elements. Although the first electrode 21₁ is disposed between the charge accumulation electrode 24₁₁ and the charge accumulation electrode 24₁₂ in the example illustrated in FIG. 59, it is possible to further improve sensitivity by disposing one first electrode 21₁ to face the two charge accumulation electrodes 24₁₁ and 24₁₂ aligned side by side as in the example illustrated in FIG. 61.

Although the present disclosure has been described on the basis of the preferable examples, the present disclosure is not limited to these examples. The structures, the configurations, the manufacturing conditions, the manufacturing methods, and the used materials of the imaging elements, the stacked imaging elements, and the solid-state imaging devices described in the examples are illustrative examples and can be appropriately changed. It is possible to appropriately combine the imaging elements in the examples. It is possible to apply the configurations and the structures of the imaging elements according to the present disclosure to a light emitting element, for example, an organic EL element, and also to apply them to a channel formation region of a thin film transistor.

It is also possible to share floating diffusion layers FD₁, FD₂, FD₃, 51C, 45C, and 46C as described above in some cases.

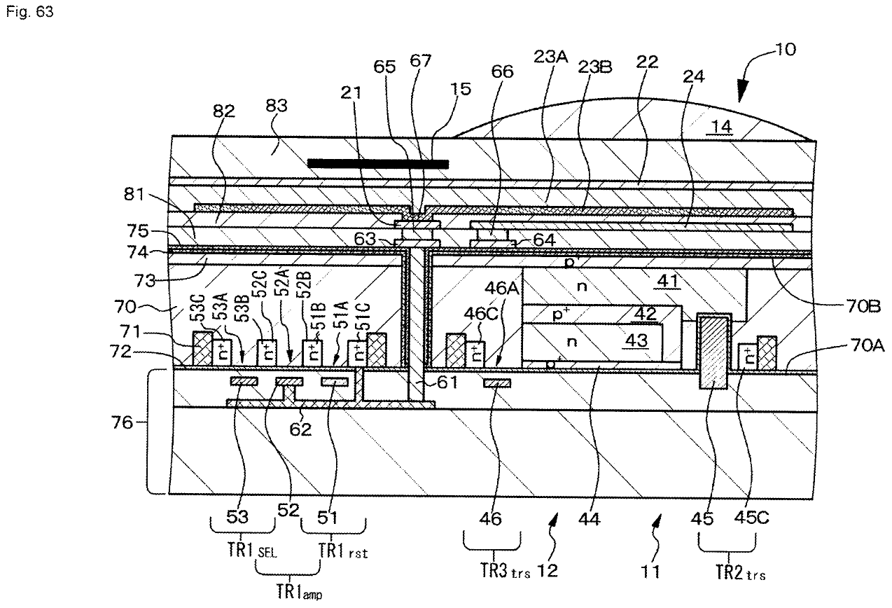

As illustrated as a modification example of the imaging element and the stacked imaging element described above in Example 1, for example, in FIG. 63, it is also possible to employ a configuration in which light is incident from the side of the second electrode 22 and a light shielding layer 15 is formed on the light incident side beyond the second electrode 22. Note that various wirings provided on the light incident side beyond the photoelectric conversion layer can also be caused to function as a light shielding layer.

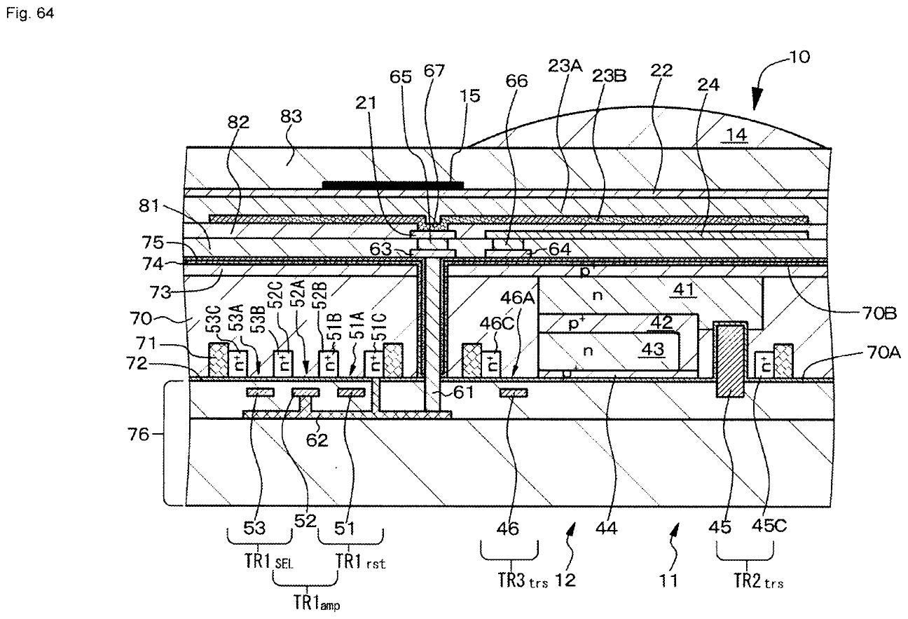

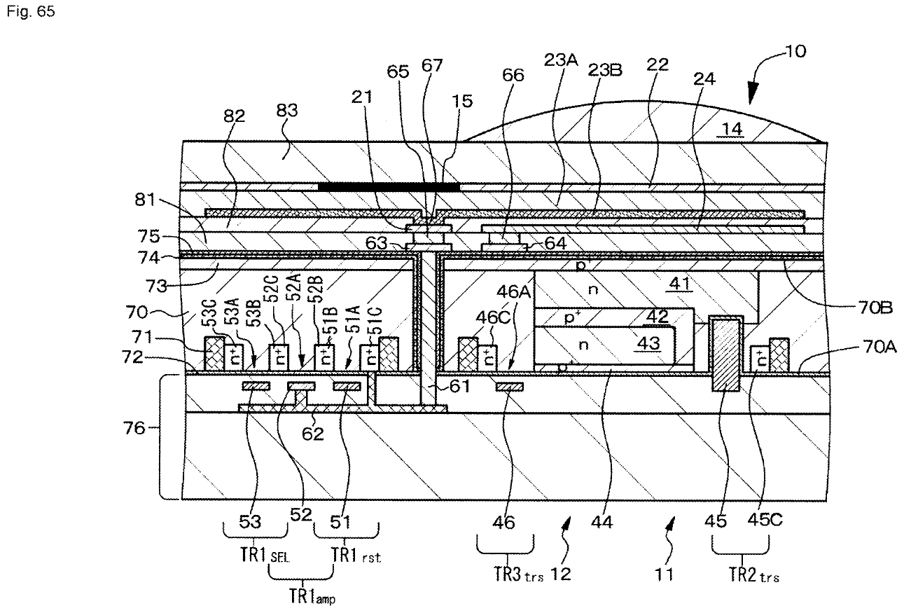

Note that although the light shielding layer 15 is formed above the second electrode 22 in the example illustrated in FIG. 63, that is, the light shielding layer 15 is formed on the light incident side close to the second electrode 22 and above the first electrode 21, the light shielding layer 15 may be disposed on a surface of the second electrode 22 on the light incident side as illustrated in FIG. 64. In some cases, the light shielding layer 15 may be formed at the second electrode 22 as illustrated in FIG. 65.

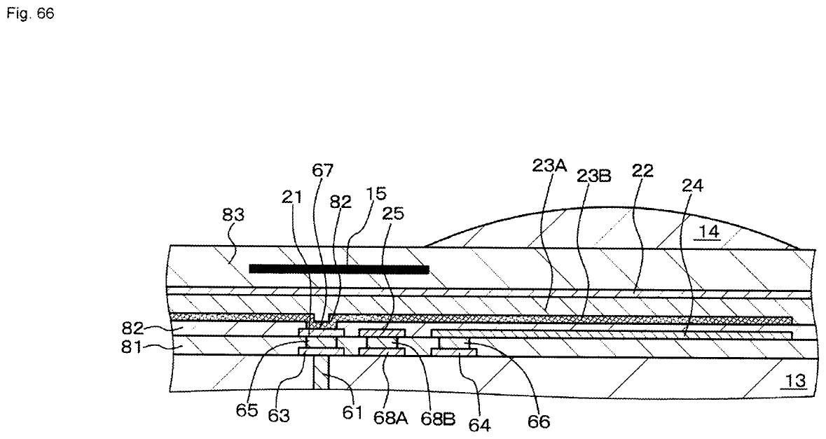

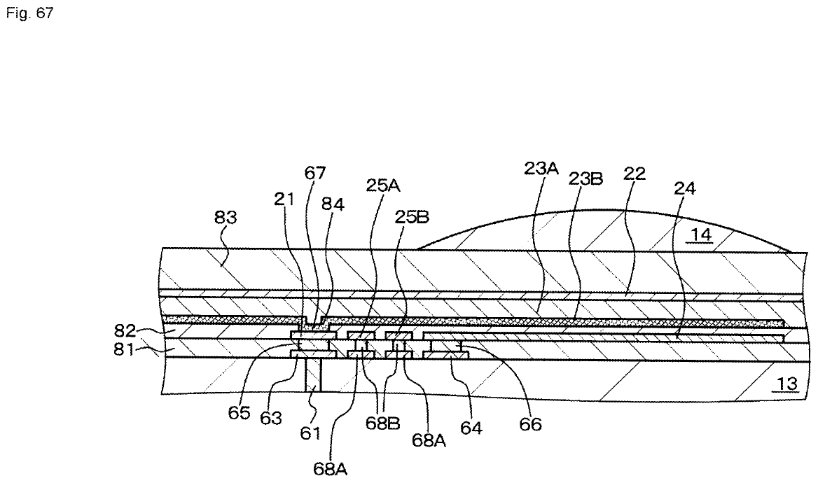

Alternatively, it is also possible to employ a structure in which light is incident from the side of the second electrode 22 and no light is incident on the first electrode 21. Specifically, the light shielding layer 15 is formed on the light incident side close to the second electrode 22 and above the first electrode 21, as illustrated in FIG. 63. Alternatively, it is also possible to employ a structure in which the on-chip micro lens 14 is provided above the charge accumulation electrode 24 and the second electrode 22 and light that is incident on the on-chip micro lens 14 is collected by the charge accumulation electrode 24 and does not reach the first electrode 21, as illustrated in FIG. 67. Note that in a case in which the transfer control electrode 25 is provided as described above in Example 4, it is possible to employ a mode in which light is not incident on the first electrode 21 and the transfer control electrode 25, and specifically, it is possible to employ a structure in which the light shielding layer 15 is formed above the first electrode 21 and the transfer control electrode 25 as illustrated in FIG. 66. Alternatively, it is possible to employ a structure in which the light that is incident on the on-chip micro lens 14 does not reach the first electrode 21 or the first electrode 21 and the transfer control electrode 25.

By employing these configurations and structures, or by providing the light shielding layer 15 or designing the on-chip micro lens 14 such that light is incident only on a portion of the photoelectric conversion unit located above the charge accumulation electrode 24, the portion of the photoelectric conversion unit located above the first electrode 21 (or above the first electrode 21 and the transfer control electrode 25) does not contribute to photoelectric conversion, and it is thus possible to more reliably reset all the pixels at once and to further easily realize the global shutter function. In other words, the method of driving a solid-state imaging device including a plurality of imaging elements including these configurations and structures includes: repeating each of steps of discharging charge in the first electrode 21 to outside of the system while accumulating charge in the inorganic oxide semiconductor material layer 23B and the like at once in all the imaging elements, then transferring the charge accumulated in the inorganic oxide semiconductor material layer 23B and the like to the first electrode 21 at once in all the imaging elements, and after the transfer is completed, successively reading the charge that has been transferred to the first electrode 21 in each imaging element.

In such a method for driving a solid-state imaging device, each imaging element has a structure in which light that is incident from the side of the second electrode is not incident on the first electrode, and the charge in the first electrode is discharged to the outside of the system while charge is accumulated in the inorganic oxide semiconductor material layer or the like at once in all the imaging elements, and it is thus possible to reliably reset the first electrodes at the same time in all the imaging elements. Thereafter, the charge accumulated in the inorganic oxide semiconductor material layer or the like is transferred to the first electrodes at once in all the imaging elements, and after the transfer is completed, the charge that has been transferred to the first electrode in each imaging element is successively read. It is thus possible to easily realize the so-called global shutter function.

In a case in which one inorganic oxide semiconductor material layer 23B shared by the plurality of imaging elements is formed, it is desirable that end portions of the inorganic oxide semiconductor material layer 23B be covered at least with the photoelectric conversion layer 23A in terms of protection of the end portions of the inorganic oxide semiconductor material layer 23B. It is only necessary for a structure of the imaging elements in such a case to be a structure illustrated at the right end of the inorganic oxide semiconductor material layer 23B with the schematic sectional view illustrated in FIG. 1.

As a modification example of Example 4, a plurality of transfer control electrodes may be provided from a position that is closest to the first electrode 21 toward the charge accumulation electrode 24 as illustrated in FIG. 67. Note that FIG. 67 illustrates an example in which two transfer control electrodes 25A and 25B are provided. An on-chip micro lens 14 is provided above the charge accumulation electrode 24 and the second electrode 22, and it is also possible to employ a structure in which light that is incident on the on-chip micro lens 14 is collected by the charge accumulation electrode 24 and does not reach the first electrode 21 and the transfer control electrodes 25A and 25B.

It is also possible to employ a configuration in which the first electrode 21 extends in an opening portion 84 provided in the insulating layer 82 and is connected to the inorganic oxide semiconductor material layer 23B.

Although in the examples, the case in which the present disclosure is applied to a CMOS type solid-state imaging device configured such that unit pixels detecting signal charge corresponding to the amount of incident light as a physical amount are disposed in a matrix has been described as an example, the present disclosure is not limited to the application to the CMOS type solid-state imaging device and can also be applied to a CCD type solid-state imaging device. In the latter case, the signal charge is transferred in a vertical direction by a vertical transfer register having a CCD type structure and is transferred in a horizontal direction by a horizontal transfer register, and a pixel signal (image signal) is output by the amplification of the signal charge. In addition, the present disclosure is not limited to all column type solid-state imaging devices in which pixels are formed in a two-dimensional matrix, and column signal processing circuits are disposed for each pixel column. Further, in some cases, a selection transistor can also be omitted.

Further, the imaging element and the stacked imaging element of the present disclosure are not limited to the application to a solid-state imaging device that detects a distribution of the amount of incident light of visible light and captures the distribution as an image, and can also be applied to a solid-state imaging device that captures a distribution of the amount of incident light of infrared light, X-rays, particles, or the like as an image. Further, in a broad sense, the present disclosure can be applied to all solid-state imaging devices (physical amount distribution detection devices) such as a fingerprint detection sensor that detects distributions of other physical amounts such as pressure and capacitance and captures the distributions as images.

Further, the present disclosure is not limited to a solid-state imaging device that scans unit pixels in an imaging region in order in units of rows and reads pixel signals from the unit pixels. The present disclosure can also be applied to an X-Y address type solid-state imaging device that selects any pixels in units of pixels and reads out pixel signals in units of pixels from the selected pixels. A solid-state imaging device may be formed as an one chip, or may be configured to have a module shape having an imaging function in which an imaging region and a drive circuit or an optical system are packaged together.

In addition, the present disclosure is not limited to the application to a solid-state imaging device and can also be applied to an imaging device. Here, the imaging device indicates a camera system such as a digital still camera or a video camera, and electronic equipment having an imaging function such as a mobile phone. A module type mounted in electronic equipment, that is, a camera module may also be used as an imaging device.

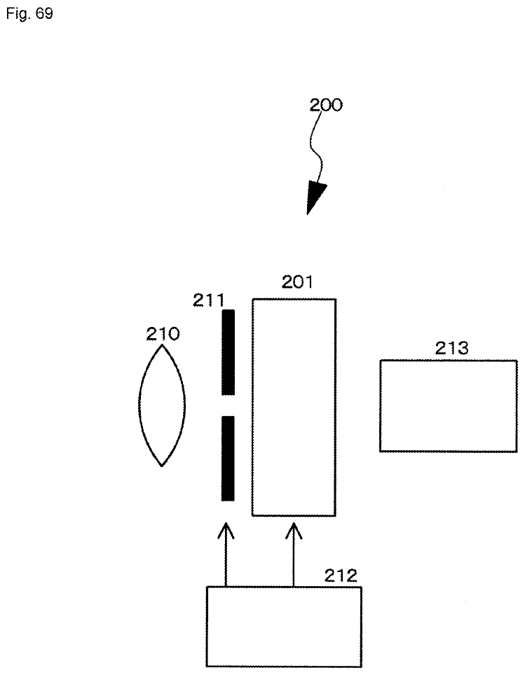

An example in which a solid-state imaging device 201 configured of the imaging element and the stacked imaging element according to the present disclosure is used in electronic equipment (camera) 200 is illustrated as a conceptual diagram in FIG. 69. The electronic equipment 200 includes the solid-state imaging device 201, an optical lens 210, a shutter device 211, a drive circuit 212, and a signal processing circuit 213. The optical lens 210 forms an image of image light (incident light) from a subject on an imaging surface of the solid-state imaging device 201. Thus, signal charges are accumulated in the solid-state imaging device 201 for a certain period of time. The shutter device 211 controls a light irradiation period and a light blocking period for the solid-state imaging device 201. The drive circuit 212 supplies a drive signal for controlling a transfer operation of the solid-state imaging device 201 and a shutter operation of the shutter device 211. Signal transfer of the solid-state imaging device 201 is performed by the drive signal (timing signal) supplied from the drive circuit 212. The signal processing circuit 213 performs various signal processing. A video signal that has undergone signal processing is stored in a storage medium such as a memory, or is output to a monitor. In such electronic equipment 200, miniaturization of a pixel size of the solid-state imaging device 201 and an improvement in transfer efficiency can be achieved, and thus it is possible to obtain the electronic equipment 200 with improved pixel characteristics. The electronic equipment 200 to which the solid-state imaging device 201 is applicable is not limited to a camera, and can also be applied to imaging devices such as a digital still camera and a camera module for mobile equipment such as a mobile phone.

The technology (the present technology) according to the present disclosure can be applied to various products. For example, the technology according to the present disclosure may be realized as a device mounted on any type of moving body such as an automobile, an electric vehicle, a hybrid electric vehicle, a motorcycle, a bicycle, a personal mobility, an airplane, a drone, a ship, and a robot.

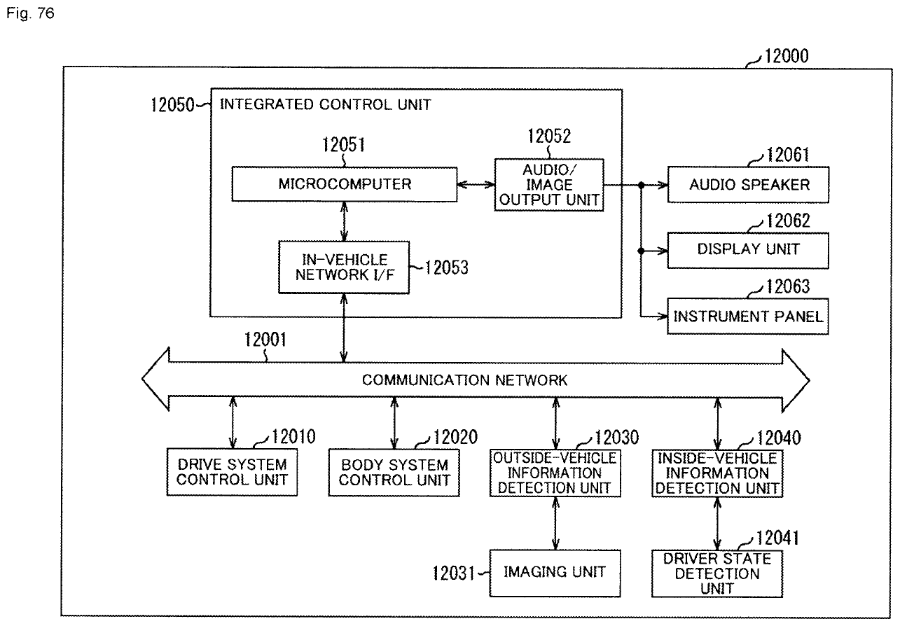

FIG. 76 is a block diagram illustrating a schematic configuration example of a vehicle control system that is an example of a mobile object control system to which the technology according to the present disclosure can be applied.

The vehicle control system 12000 includes a plurality of electronic control units connected via a communication network 12001. In the example illustrated in FIG. 76, the vehicle control system 12000 includes a drive system control unit 12010, a body system control unit 12020, an outside-vehicle information detection unit 12030, an inside-vehicle information detection unit 12040, and an integrated control unit 12050. In addition, as a functional configuration of the integrated control unit 12050, a microcomputer 12051, an audio/image output unit 12052, and an in-vehicle network interface (I/F) 12053 are illustrated.

The drive system control unit 12010 controls operations of devices related to a drive system of a vehicle according to various programs. For example, the drive system control unit 12010 functions as a control device of a driving force generation device for generating a driving force of a vehicle such as an internal combustion engine or a driving motor, a driving force transmission mechanism for transmitting a driving force to wheels, a steering mechanism for adjusting a turning angle of a vehicle, and a braking device that generates a braking force of a vehicle, and the like.

The body system control unit 12020 controls operations of various devices mounted in the vehicle body according to various programs. For example, the body system control unit 12020 functions as a control device such as a keyless entry system, a smart key system, a power window device, or various lamps such as a headlamp, a back lamp, a brake lamp, a turn signal and a fog lamp. In this case, radio waves transmitted from a portable device that substitutes for a key or signals of various switches may be input to the body system control unit 12020. The body system control unit 12020 receives inputs of the radio waves or signals, and controls a door lock device, a power window device, and a lamp of the vehicle.

The outside-vehicle information detection unit 12030 detects information outside the vehicle in which the vehicle control system 12000 is mounted. For example, an imaging unit 12031 is connected to the outside-vehicle information detection unit 12030. The outside-vehicle information detection unit 12030 causes the imaging unit 12031 to capture an image of the outside of the vehicle and receives the captured image. The outside-vehicle information detection unit 12030 may perform object detection processing or distance detection processing for peoples, cars, obstacles, signs, and letters on the road on the basis of the received image.

The imaging unit 12031 is an optical sensor that receives light and outputs an electrical signal according to the amount of received light. The imaging unit 12031 can also output the electrical signal as an image and ranging information. In addition, the light received by the imaging unit 12031 may be visible light or invisible light such as infrared light.

The inside-vehicle information detection unit 12040 detects information on the inside of the vehicle. For example, a driver state detection unit 12041 that detects a driver's state is connected to the inside-vehicle information detection unit 12040. The driver state detection unit 12041 includes, for example, a camera that captures an image of a driver, and the inside-vehicle information detection unit 12040 may calculate a degree of fatigue or concentration of the driver or may determine whether or not the driver is dozing on the basis of detection information input from the driver state detection unit 12041.

The microcomputer 12051 can calculate a control target value of the driving force generation device, the steering mechanism, or the braking device on the basis of the information on the inside and the outside of the vehicle acquired by the outside-vehicle information detection unit 12030 or the inside-vehicle information detection unit 12040, and output a control command to the drive system control unit 12010. For example, the microcomputer 12051 can perform cooperative control in order to realize functions of an advanced driver assistance system (ADAS) such as vehicle collision avoidance or impact mitigation, following traveling based on an inter-vehicle distance, vehicle speed maintenance traveling, vehicle collision warning, and vehicle lane deviation warning.

Further, the microcomputer 12051 can perform cooperative control for the purpose of automated driving or the like in which autonomous travel is performed without depending on operations of the driver by controlling the driving force generation device, the steering mechanism, the braking device, and the like on the basis of information regarding the surroundings of the vehicle acquired by the outside-vehicle information detection unit 12030 or the inside-vehicle information detection unit 12040.

In addition, the microcomputer 12051 can output a control command to the body system control unit 12020 based on the information outside the vehicle acquired by the outside-vehicle information detection unit 12030. For example, the microcomputer 12051 can perform cooperative control for the purpose of preventing glare, such as switching from a high beam to a low beam, by controlling the headlamp according to the position of a preceding vehicle or an oncoming vehicle detected by the outside-vehicle information detection unit 12030.

The audio/image output unit 12052 transmits an output signal of at least one of audio and an image to an output device capable of visually or audibly notifying an occupant of a vehicle or the outside of the vehicle of information. In the example illustrated in FIG. 76, as such an output device, an audio speaker 12061, a display unit 12062 and an instrument panel 12063 are illustrated. The display unit 12062 may include, for example, at least one of an onboard display and a head-up display.

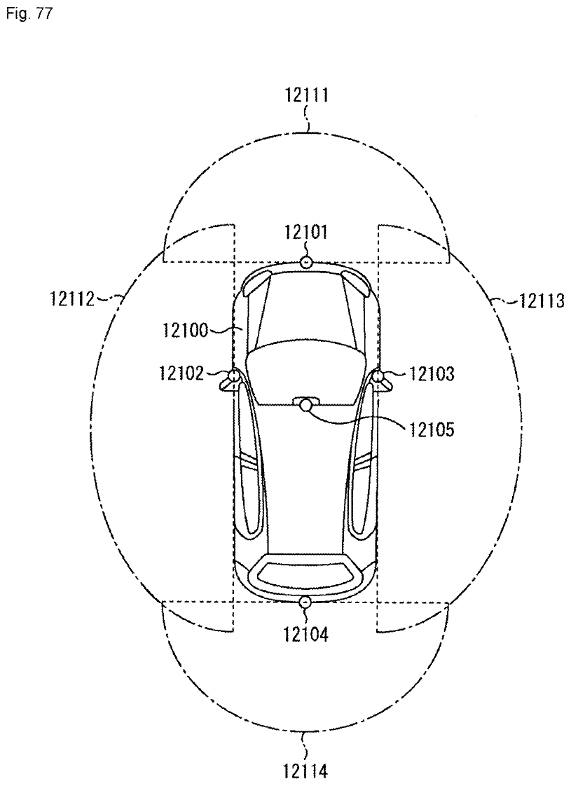

FIG. 77 is a diagram illustrating an example of an installation position of the imaging unit 12031.

In FIG. 77, a vehicle 12100 includes imaging units 12101, 12102, 12103, 12104, and 12105 as the imaging unit 12031.

The imaging units 12101, 12102, 12103, 12104, and 12105 may be provided at positions such as a front nose, side-view mirrors, a rear bumper, a back door, and an upper portion of a vehicle interior front glass of the vehicle 12100, for example. The imaging unit 12101 provided on a front nose and the imaging unit 12105 provided in an upper portion of the vehicle interior front glass mainly acquire images of a side in front of the vehicle 12100. The imaging units 12102 and 12103 provided on the side mirrors mainly acquire images of sides of the vehicle 12100. The imaging unit 12104 provided on the rear bumper or the back door mainly acquires images of a side behind the vehicle 12100. The images of a front side which are acquired by the imaging units 12101 and 12105 are mainly used for detection of preceding vehicles, pedestrians, obstacles, traffic signals, traffic signs, lanes, and the like.

Note that FIG. 77 illustrates an example of imaging ranges of the imaging units 12101 to 12104. An imaging range 12111 indicates an imaging range of the imaging unit 12101 provided at the front nose, imaging ranges 12112 and 12113 respectively indicate the imaging ranges of the imaging units 12102 and 12103 provided at the side mirrors, and an imaging range 12114 indicates the imaging range of the imaging unit 12104 provided at the rear bumper or the back door. For example, a bird's-eye view image of the vehicle 12100 as viewed from above can be obtained by superimposition of image data captured by the imaging units 12101 to 12104.

At least one of the imaging units 12101 to 12104 may have a function for acquiring distance information. For example, at least one of the imaging units 12101 to 12104 may be a stereo camera constituted by a plurality of imaging elements or may be an imaging element that has pixels for phase difference detection.

For example, the microcomputer 12051 can extract, particularly, a closest three-dimensional object on a path through which the vehicle 12100 is traveling, which is a three-dimensional object traveling at a predetermined speed (for example, 0 km/h or higher) in the substantially same direction as the vehicle 12100, as a preceding vehicle by acquiring a distance to each of three-dimensional objects in the imaging ranges 12111 to 12114 and temporal change in the distance (a relative speed with respect to the vehicle 12100) on the basis of distance information obtained from the imaging units 12101 to 12104. Further, the microcomputer 12051 can set an inter-vehicle distance which should be guaranteed in advance in front of a preceding vehicle and can perform automated brake control (also including following stop control) or automated acceleration control (also including following start control). In this manner, it is possible to perform cooperated control in order to perform automated driving or the like in which a vehicle autonomously travels irrespective of a manipulation of a driver.

For example, the microcomputer 12051 can classify and extract three-dimensional object data regarding three-dimensional objects into two-wheeled vehicles, ordinary vehicles, large vehicles, pedestrians, and other three-dimensional objects such as utility poles on the basis of distance information obtained from the imaging units 12101 to 12104 and use the three-dimensional object data for automatic avoidance of obstacles. For example, the microcomputer 12051 identifies obstacles in the vicinity of the vehicle 12100 into obstacles that can be visually recognized by the driver of the vehicle 12100 and obstacles that are difficult to visually recognize. Then, the microcomputer 12051 can determine a risk of collision indicating the degree of risk of collision with each obstacle, and can perform driving assistance for collision avoidance by outputting a warning to a driver through the audio speaker 12061 or the display unit 12062 and performing forced deceleration or avoidance steering through the drive system control unit 12010 when the risk of collision has a value equal to or greater than a set value and there is a possibility of collision.

At least one of the imaging units 12101 to 12104 may be an infrared camera that detects infrared rays. For example, the microcomputer 12051 can recognize a pedestrian by determining whether there is a pedestrian in the captured image of the imaging units 12101 to 12104. Such pedestrian recognition is performed by, for example, a procedure in which feature points in the captured images of the imaging units 12101 to 12104 as infrared cameras are extracted and a procedure in which pattern matching processing is performed on a series of feature points indicating the outline of the object and it is determined whether the object is a pedestrian. When the microcomputer 12051 determines that there is a pedestrian in the captured images of the imaging units 12101 to 12104, and the pedestrian is recognized, the audio/image output unit 12052 controls the display unit 12062 so that the recognized pedestrian is superimposed and displayed with a square contour line for emphasis. In addition, the audio/image output unit 12052 may control the display unit 12062 so that an icon indicating a pedestrian or the like is displayed at a desired position.

In addition, for example, the technology according to the present disclosure may be applied to an endoscopic surgery system.

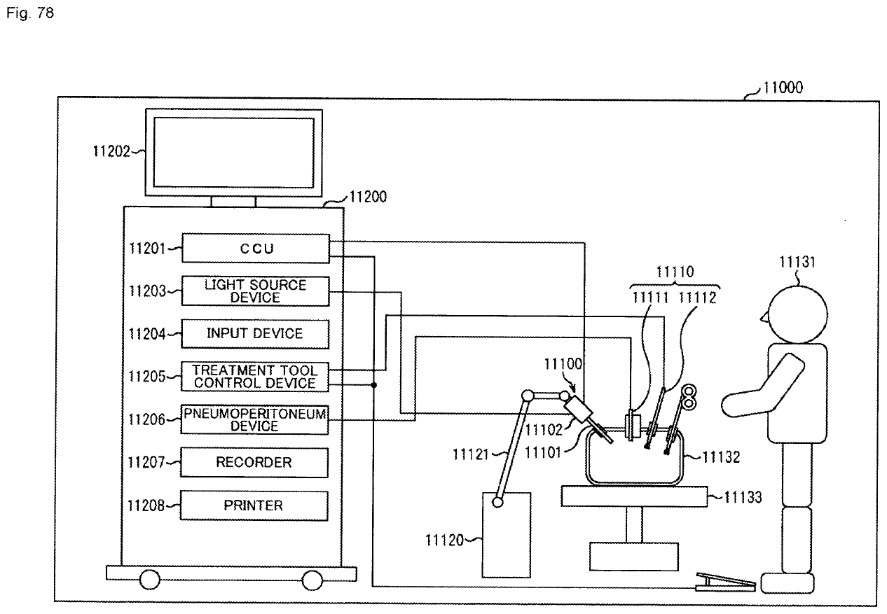

FIG. 78 is a diagram illustrating an example of a schematic configuration of an endoscopic surgery system to which the technology according to the present disclosure (the present technology) can be applied.

FIG. 78 illustrates a state where a surgeon (doctor) 11131 is performing a surgical operation on a patient 11132 on a patient bed 11133 by using the endoscopic surgery system 11000. As illustrated in the drawing, the endoscopic surgery system 11000 includes an endoscope 11100, other surgical instruments 11110 such as a pneumoperitoneum tube 11111 and an energized treatment tool 11112, a support arm device 11120 that supports the endoscope 11100, and a cart 11200 equipped with various devices for endoscopic operation.

The endoscope 11100 includes a lens barrel 11101, a region of which with a predetermined length from a distal end is inserted into a body cavity of the patient 11132, and a camera head 11102 connected to a proximal end of the lens barrel 11101. Although the endoscope 11100 configured as a so-called rigid mirror having the rigid lens barrel 11101 is illustrated in the illustrated example, the endoscope 11100 may be configured as a so-called flexible mirror having a flexible lens barrel.

An opening in which an objective lens is fitted is provided at the distal end of the lens barrel 11101. A light source device 11203 is connected to the endoscope 11100, and light generated by the light source device 11203 is guided to the distal end of the lens barrel by a light guide extending inside the lens barrel 11101 and is radiated toward the observation target in the body cavity of the patient 11132 via the objective lens. The endoscope 11100 may be a direct-viewing endoscope or may be a perspective endoscope or a side-viewing endoscope.

An optical system and an imaging element are provided inside the camera head 11102, and the reflected light (observation light) from the observation target converges on the imaging element by the optical system. The observation light is photoelectrically converted by the imaging element, and an electrical signal corresponding to the observation light, that is, an image signal corresponding to an observation image is generated. The image signal is transmitted as RAW data to a camera control unit (CCU) 11201.

The CCU 11201 is composed of a central processing unit (CPU), a graphics processing unit (GPU) or the like, and comprehensively controls the operation of the endoscope 11100 and a display device 11202. In addition, the CCU 11201 receives an image signal from the camera head 11102, and performs various kinds of image processing for displaying an image based on the image signal, for example, development processing (demosaic processing) on the image signal.

The display device 11202 displays an image based on an image signal having been subjected to image processing by the CCU 11201 under the control of the CCU 11201.

The light source device 11203 is constituted by, for example, a light source such as a light emitting diode (LED), and supplies irradiation light at the time of imaging a surgical part or the like to the endoscope 11100.

The input device 11204 is an input interface for the endoscopic surgery system 11000. The user can input various kinds of information or instructions to the endoscopic surgery system 11000 via the input device 11204. For example, the user inputs an instruction to change imaging conditions (a type of irradiation light, a magnification, a focal length, or the like) of the endoscope 11100.

A treatment tool control device 11205 controls the driving of an energized treatment tool 11112 for cauterizing or incising tissue, sealing a blood vessel, or the like. In order to secure a field of view of the endoscope 11100 and secure an operation space of the surgeon, a pneumoperitoneum device 11206 sends gas into the body cavity of the patient 11132 via the pneumoperitoneum tube 11111 in order to inflate the body cavity. A recorder 11207 is a device that can record various kinds of information related to surgery. A printer 11208 is a device that can print various kinds of information related to surgery in various formats such as text, images and graphs.

The light source device 11203 that supplies the endoscope 11100 with the irradiation light for imaging the surgical part can be configured of, for example, an LED, a laser light source, or a white light source configured of a combination thereof. When a white light source is formed by a combination of RGB laser light sources, it is possible to control an output intensity and an output timing of each color (each wavelength) with high accuracy and thus, the light source device 11203 can adjust white balance of the captured image. Further, in this case, laser light from each of the respective RGB laser light sources is radiated to the observation target in a time division manner, and driving of the imaging element of the camera head 11102 is controlled in synchronization with radiation timing such that images corresponding to respective RGB can be captured in a time division manner. According to this method, it is possible to obtain a color image without providing a color filter to the imaging element.

Further, the driving of the light source device 11203 may be controlled to change the intensity of output light at predetermined time intervals. The driving of the imaging element of the camera head 11102 is controlled in synchronization with the timing of the change in the light intensity to acquire an image in a time-division manner, and the image is synthesized, whereby it is possible to generate a so-called image in a high dynamic range without underexposure or overexposure.

In addition, the light source device 11203 may have a configuration in which light in a predetermined wavelength band corresponding to special light observation can be supplied. In the special light observation, for example, by emitting light in a band narrower than that of irradiation light (that is, white light) during normal observation using wavelength dependence of light absorption in a body tissue, so-called narrow band light observation (narrow band imaging) in which a predetermined tissue such as a blood vessel in the mucous membrane surface layer is imaged with a high contrast is performed. Alternatively, in the special light observation, fluorescence observation in which an image is obtained by fluorescence generated by emitting excitation light may be performed. The fluorescence observation can be performed by emitting excitation light to a body tissue, and observing fluorescence from the body tissue (autofluorescence observation), or locally injecting a reagent such as indocyanine green (ICG) to a body tissue, and emitting excitation light corresponding to a fluorescence wavelength of the reagent to the body tissue to obtain a fluorescence image. The light source device 11203 can supply narrow band light and/or excitation light corresponding to such special light observation.

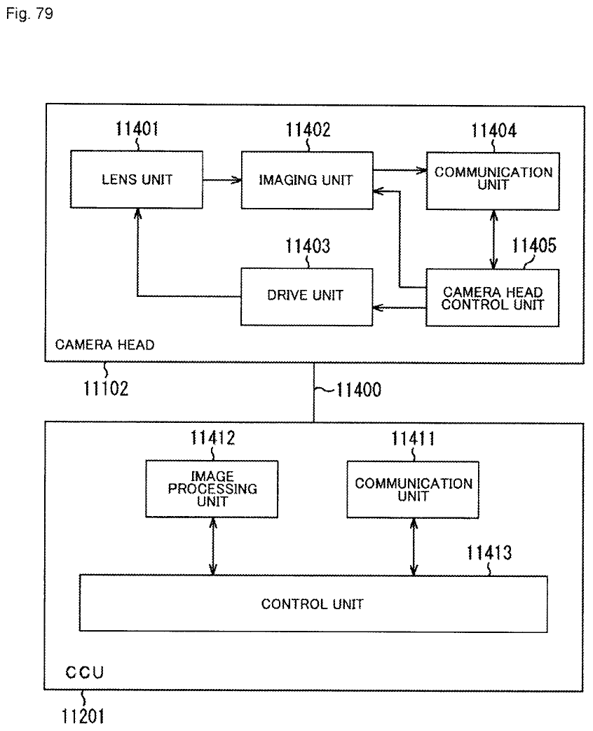

FIG. 79 is a block diagram illustrating an example of functional configurations of the camera head 11102 and the CCU 11201 illustrated in FIG. 78.

The camera head 11102 includes a lens unit 11401, an imaging unit 11402, a drive unit 11403, a communication unit 11404, and a camera head control unit 11405. The CCU 11201 includes a communication unit 11411, an image processing unit 11412, and a control unit 11413. The camera head 11102 and the CCU 11201 are connected to each other such that they can communicate with each other via a transmission cable 11400.

The lens unit 11401 is an optical system provided at a portion connected to the lens barrel 11101. Observation light taken from the tip of the lens barrel 11101 is guided to the camera head 11102 and is incident on the lens unit 11401. The lens unit 11401 is constituted by a combination of a plurality of lenses including a zoom lens and a focus lens.

The imaging unit 11402 is constituted by an imaging element. The imaging element constituting the imaging unit 11402 may be one element (so-called single plate type) or a plurality of elements (so-called multi-plate type). When the imaging unit 11402 is configured as a multi-plate type, image signals corresponding to RGB are generated by the imaging elements, and a color image may be obtained by synthesizing the image signals, for example. Alternatively, the imaging unit 11402 may be configured to include a pair of imaging elements for acquiring image signals for the right eye and the left eye corresponding to three-dimensional (3D) display. When 3D display is performed, the surgeon 11131 can ascertain the depth of biological tissues in the surgical part more accurately. Note that in a case in which the imaging unit 11402 is configured as a multi-plate type, a plurality of lens units 11401 may be provided according to the imaging elements.

Further, the imaging unit 11402 may not necessarily be provided in the camera head 11102. For example, the imaging unit 11402 may be provided immediately after the objective lens inside the lens barrel 11101.

The drive unit 11403 is constituted by an actuator, and moves the zoom lens and the focus lens of the lens unit 11401 by a predetermined distance along an optical axis under the control of the camera head control unit 11405. The magnification and the focus of the image captured by the imaging unit 11402 can thus be appropriately adjusted.

The communication unit 11404 is configured of a communication device for transmitting or receiving various information to or from the CCU 11201. The communication unit 11404 transmits the image signal obtained from the imaging unit 11402 as RAW data to the CCU 11201 via the transmission cable 11400.

In addition, the communication unit 11404 receives a control signal for controlling driving of the camera head 11102 from the CCU 11201 and supplies the control signal to the camera head control unit 11405. The control signal includes, for example, information on the imaging conditions such as information indicating that the frame rate of the captured image is designated, information indicating that the exposure value at the time of imaging is designated, and/or information indicating that the magnification and the focus of the captured image are designated.

Note that the imaging conditions such as the frame rate, the exposure value, the magnification, and the focus may be appropriately designated by the user, or may be automatically set by the control unit 11413 of the CCU 11201 on the basis of the acquired image signal. In the latter case, a so-called auto exposure (AE) function, auto focus (AF) function and auto white balance (AWB) function are provided in the endoscope 11100.

The camera head control unit 11405 controls the driving of the camera head 11102 on the basis of the control signal from the CCU 11201 received via the communication unit 11404.

The communication unit 11411 is constituted by a communication device for transmitting and receiving various pieces of information to and from the camera head 11102. The communication unit 11411 receives the image signal transmitted from the camera head 11102 via the transmission cable 11400.

In addition, the communication unit 11411 transmits a control signal for controlling the driving of the camera head 11102 to the camera head 11102. The image signal or the control signal can be transmitted through electric communication, optical communication, or the like.

The image processing unit 11412 performs various image processing on the image signal which is the RAW data transmitted from the camera head 11102.

The control unit 11413 performs various kinds of control regarding imaging of an operation site or the like using the endoscope 11100 and a display of a captured image obtained by imaging the operation site or the like. For example, the control unit 11413 generates the control signal for controlling the driving of the camera head 11102.

Further, the control unit 11413 causes the display device 11202 to display the captured image obtained by imaging the operation site or the like on the basis of the image signal having subjected to image processing by the image processing unit 11412. At this time, the control unit 11413 may recognize various objects in the captured image using various image recognition technologies. For example, the control unit 11413 can recognize surgical tools such as forceps, specific biological parts, bleeding, mist when the energized treatment tool 11112 is used and the like by detecting the edge shape and color of the object included in the captured image. When the control unit 11413 causes the display device 11202 to display the captured image, it may cause various kinds of surgical support information to be superimposed and displayed with the image of the operation site using the recognition result. When the surgical support information is superimposed and displayed, and presented to the surgeon 11131, it is possible to reduce the burden on the surgeon 11131 and the surgeon 11131 can reliably proceed the operation.

The transmission cable 11400 connecting the camera head 11102 and the CCU 11201 to each other is an electric signal cable that supports electric signal communication, an optical fiber that supports optical communication, or a composite cable thereof.

Here, although communication is performed in a wired manner using the transmission cable 11400 in the example illustrated in the drawing, communication between the camera head 11102 and the CCU 11201 may be performed in a wireless manner.

Although the endoscopic surgery system has been described as an example here, the technology according to the present disclosure may be applied to other, for example, a microscopic operation system.

Note that the present disclosure can also adopt the following configurations.

### [A01]<<Imaging Element>>

An imaging element including: a photoelectric conversion unit that is configured of a first electrode and a photoelectric conversion layer containing an organic material and a second electrode being laminated, in which an inorganic oxide semiconductor material layer is formed between the first electrode and the photoelectric conversion layer, and an inorganic oxide semiconductor material configuring the inorganic oxide semiconductor material layer contains gallium atoms, tin atoms, zinc atoms, and oxygen atoms.

[A02] The imaging element according to [A01], in which an optical gap of the inorganic oxide semiconductor material is equal to or greater than 2.7 eV and equal to or less than 3.2 eV.
[A03] The imaging element according to [A02], in which when a composition of the inorganic oxide semiconductor material is represented as GaₐSn_{b}Zn_{c}O_{d} (where a+b+c=1.00, and a>0, b>0, and c>0), 0.45(b−0.62)≤0.55a≤0.45b . . . (1) is satisfied.
[A04] The imaging element according to any one of [A01] to [A03], in which oxygen deficiency generation energy of the inorganic oxide semiconductor material is equal to or greater than 2.6 eV.
[A05] The imaging element according to [A04], in which when a composition of the inorganic oxide semiconductor material is represented as GaₐSn_{b}Zn_{c}O_{d} (where a+b+c=1.00, and a>0, b>0, and c>0), a≤−3.0(b−0.63) . . . (2) is satisfied.
[A06] The imaging element according to any one of [A01] to [A05], in which carrier mobility of the inorganic oxide semiconductor material layer is equal to or greater than 10 cm²/V·s, and when a composition of the inorganic oxide semiconductor material is represented as GaₐSn_{b}Zn_{c}O_{d} (where a+b+c=1.00, and a>0, b>0, and c>0), b>0.23 . . . (3) is satisfied.
[A07] The imaging element according to [A01], in which when a composition of an inorganic oxide semiconductor material is represented as GaₐSn_{b}Zn_{c}O_{d} (where a+b+c=1.00, and a>0, b>0, and c>0), values of a, b, and c satisfy Expression (1) below, or satisfy Expression (2) below, or satisfy Expression (3) below, or satisfy Expressions (1) and (2) below, satisfy Expressions (1) and (3) below, or satisfy Expressions (2) and (3) below, or satisfy Expressions (1), (2), and (3) below. Here,

0.45(b−0.62)≤0.55a≤0.45b (1)

a≤−3.0(b−0.63) (2)

b≥0.23 (3)

[A08] The imaging element according to [A01], in which when a composition of the inorganic oxide semiconductor material is represented as GaₐSn_{b}Zn_{c}O_{d} (where a+b+c=1.00, and a>0, b>0, and c>0), values of a, b, and c satisfy all of Expressions (1), (2), and (3) below.

0.45(b−0.62)≤0.55a≤0.45b (1)

a≤−3.0(b−0.63) (2)

b≥0.23 (3)

[A09] The imaging element according to [A01], in which when a composition of the inorganic oxide semiconductor material is represented as GaₐSn_{b}Zn_{c}O_{d} (where a+b+c=1.00, and a>0, b>0, and c>0), b>a and b>c are satisfied. [A10] The imaging element according to any one of [A01] to [A09], in which carrier concentration of the inorganic oxide semiconductor material layer is equal to or greater than 1×10¹⁴ cm⁻³ and equal to or less than 1×10¹⁷ cm⁻³.
[A11] The imaging element according to any one of [A01] to [A10], in which carrier mobility of the inorganic oxide semiconductor material layer is equal to or greater than 10 cm²/V·s.
[A12] The imaging element according to any one of [A01] to [A11], in which the photoelectric conversion unit includes an insulating layer and a charge accumulation electrode disposed to be separated from the first electrode and face the inorganic oxide semiconductor material layer via the insulating layer.
[A13] The imaging element according to any one of [A01] to [A12], in which charge generated in the photoelectric conversion layer is transferred to the first electrode via the inorganic oxide semiconductor material layer.
[A14] The imaging element according to [A13], in which the charge is electrons.
[A15] The imaging element according to any one of [A01] to [A14], in which a thickness of the inorganic oxide semiconductor material layer is 1×10⁻⁸ m to 1.5×10⁻⁷ m.
[A16] The imaging element according to any one of [A01] to [A15], in which the inorganic oxide semiconductor material layer is amorphous.
[A17] The imaging element according to any one of [A01] to [A16], in which light is incident from the second electrode, surface roughness Ra of a surface of the inorganic oxide semiconductor material layer at a boundary between the photoelectric conversion layer and the inorganic oxide semiconductor material layer is equal to or less than 1.5 nm, and a value of root-mean-square roughness Rq of the surface of the inorganic oxide semiconductor material layer is equal to or less than 2.5 nm.
[B01] The imaging element according to any one of [A01] to [A17], in which a value ΔEN obtained by subtracting an average value EN_{cation} of electronegativity of cation species configuring the inorganic oxide semiconductor material layer from an average value ENₐₙᵢₒₙ of electronegativity of anion species configuring the inorganic oxide semiconductor material layer is less than 1.695.
[B02] The imaging element according to [B01], in which when the inorganic oxide semiconductor material layer is represented as (A¹ₐ₁A²ₐ₂A³ₐ₃ . . . A^{M}_{aM})(B¹_{b1}B²_{b2}B³_{b3} . . . B^{N}_{bN}) [where A¹, A², A³, . . . A^{M} are cation species, B¹, B², B³, . . . B^{N} are anion species, a1, a2, a3, . . . , aM, b1, b2, b3, . . . , bN are values corresponding to atomic percentages, and a total is 1.00], representations of ENₐₙᵢₒₙ=(B1×b1+B2×b2+B3×b3 . . . +BN×bN)/(b1+b2+b3 . . . +bN) and EN_{cation}=(A1×a1+A2×a2+A3×a3 . . . +AM×aM)/(a1+a2+a3 . . . +aM) are established.

Here, B1, B2, B3, . . . , BN are electronegativity of the anion species B¹, B², B³, . . . , BN, and A1, A2, A3, . . . , AM are electronegativity of the cation species A¹, A², A³, . . . , A^{M}.

[C01] The imaging element according to any one of [A01] to [B02], in which the inorganic oxide semiconductor material layer is configured of a first layer and a second layer from a side of the first electrode, and when average film density in the first layer from a boundary between the first electrode and the inorganic oxide semiconductor material layer to 3 nm, preferably to 5 nm, or more preferably to 10 nm is defined as ρ₁ and average film density in the second layer is defined as ρ₂, ρ₁≥5.9 g/cm³ and ρ₁−ρ₂≥0.1 g/cm³ are satisfied.

[C02] The imaging element according to [C01], in which a composition of the first layer and a composition of the second layer are the same.
[C03] The imaging element according to any one of [A01] to [B02], in which the inorganic oxide semiconductor material layer is configured of a first layer and a second layer from a side of the first electrode, a composition of the first layer and a composition of the second layer are the same, and when average film density in the first layer from a boundary between the first electrode and the inorganic oxide semiconductor material layer to 3 nm, preferably to 5 nm, or more preferably to 10 nm is defined as ρ₁ and average film density in the second layer is defined as ρ₂, ρ₁−ρ₂≥0.1 g/cm³ is satisfied.
[C04] The imaging element according to any one of [C01] to [C03], in which when average oxygen deficiency generation energy of the first layer is defined as E_{OD-1′}, and average oxygen deficiency generation energy of the second layer is defined as E_{OD-2}′, E_{OD-1′}≥2.8 eV and E_{OD-1′}−E_{OD-2′}≥0.2 eV are satisfied.
[C05] The imaging element according to any one of [A01] to [C03], in which the inorganic oxide semiconductor material layer is configured of a first layer and a second layer from a side of the first electrode, and when average oxygen deficiency generation energy of the first layer from a boundary between the first electrode and the inorganic oxide semiconductor material layer to 3 nm, preferably to 5 nm, or more preferably to 10 nm is defined as E_{OD-1′}, and average oxygen deficiency generation energy of the second layer is defined as E_{OD-2′}, E_{OD-1′} >2.8 eV and E_{OD-1′}−E_{OD-2′}>0.2 eV are satisfied.
[C06] The imaging element according to [C05], in which a composition of the first layer and a composition of the second layer are the same.
[C07] The imaging element according to any one of [A01] to [C03], in which the inorganic oxide semiconductor material layer is configured of a first layer and a second layer from a side of the first electrode, a composition of the first layer and a composition of the second layer are the same, and when average oxygen deficiency generation energy of the first layer from a boundary between the first electrode and the inorganic oxide semiconductor material layer to 3 nm, preferably to 5 nm, or more preferably to 10 nm is defined as E_{OD-1′}, and average oxygen deficiency generation energy of the second layer is defined as E_{OD-2′}, E_{OD-1′} >2.8 eV and E_{OD-1′}- E_{OD-2′}>0.2 eV are satisfied.
[D01] The imaging element according to any one of [A01] to [C07], in which a protective layer configured of an inorganic oxide and the inorganic oxide semiconductor material layer are formed from a side of the photoelectric conversion unit immediately below the photoelectric conversion layer.
[D02] The imaging element according to [D01], in which oxygen deficiency generation energy of metal atoms configuring the protective layer is equal to or greater than 5 eV.
[D03] The imaging element according to [D02], in which oxygen deficiency generation energy of the metal atoms configuring the protective layer is defined as E_{OD-1}, and oxygen deficiency generation energy of metal atoms configuring the inorganic oxide semiconductor material layer is defined as E_{OD-2}, E_{OD-1}−E_{OD-2}≥1 eV is satisfied.
[D04] The imaging element according to any one of [D01] to [D03], in which the protective layer inhibits invasion of hydrogen to the inorganic oxide semiconductor material layer.
[D05] The imaging element according to [D04], in which in regard to hydrogen inhibition ability of the protective layer, a hydrogen ion relative strength ratio detected during heating at 350° C. that is measured using a thermal desorption spectroscopy method is equal to or less than 0.1 on the assumption that a hydrogen ion relative strength ratio when titanium is heated is 1.0.
[E01] The imaging element according to any one of [A01] to [D05], further including: a semiconductor substrate, in which the photoelectric conversion unit is disposed above the semiconductor substrate, and the photoelectric conversion unit further includes an insulating layer and a charge accumulation electrode that is disposed to be separated from the first electrode and face the inorganic oxide semiconductor material layer via the insulating layer.
[E02] The imaging element according to [E01], in which the first electrode extends in an opening portion provided in the insulating layer and is connected to the inorganic oxide semiconductor material layer.
[E03] The imaging element according to [E01], in which the inorganic oxide semiconductor material layer extends in an opening portion provided in the insulating layer and is connected to the first electrode.
[E04] The imaging element according to [E03], in which an edge portion of a top surface of the first electrode is covered with an insulating layer, the first electrode is exposed from a bottom surface of the opening portion, and when a surface of the insulating layer that is in contact with the top surface of the first electrode is defined as a first surface, and a surface of the insulating layer that is in contact with a portion of the inorganic oxide semiconductor material layer facing the charge accumulation electrode is defined as a second surface, a side surface of the opening portion has an inclination spreading from the first surface toward the second surface.
[E05] The imaging element according to [E04], in which the side surface of the opening portion that has the inclination spreading from the first surface toward the second surface is located on a side of the charge accumulation electrode.

### [E06] <<Control of Potentials of First Electrode and Charge Accumulation Electrode>>

The imaging element according to any one of [E01] to [E05], further including: a control unit that is provided in the semiconductor substrate and includes a drive circuit, the first electrode and the charge accumulation electrode are connected to the drive circuit, a potential V₁₁ is applied to the first electrode and a potential V₃₁ is applied to the charge accumulation electrode from the drive circuit, and charge is accumulated in the inorganic oxide semiconductor material layer (or the inorganic oxide semiconductor material layer and the photoelectric conversion layer, or the inorganic oxide semiconductor material layer, the protective layer, and the photoelectric conversion layer) in a charge accumulation period, and a potential Vie is applied to the first electrode and a potential V₃₂ is applied to the charge accumulation electrode from the drive circuit, and the charge accumulated in the inorganic oxide semiconductor material layer (or the inorganic oxide semiconductor material layer and the photoelectric conversion layer, or the inorganic oxide semiconductor material layer, the protective layer, and the photoelectric conversion layer) is read by the control unit via the first electrode in a charge transfer period.

Here, the potential of the first electrode is higher than the potential of the second electrode, and V₃₁>V₁₁ and V₃₂<V₁₂ are satisfied.

### [E07] <<Lower Charge Movement Control Electrode>>

The imaging element according to any one of [E01] to [E06], in which a lower charge movement control electrode is formed in a region that faces via an insulating layer a region of the photoelectric conversion layer located between adjacent imaging elements.

### [E08] <<Control of Potentials of First Electrode, Charge Accumulation Electrode, and Lower Charge Movement Control Electrode>>

The imaging element according to [E07], further including: a control unit that is provided in the semiconductor substrate and includes a drive circuit, the first electrode, the second electrode, the charge accumulation electrode, and the lower charge movement control electrode are connected to the drive circuit, a potential V₁₁ is applied to the first electrode and a potential V₃₁ is applied to the charge accumulation electrode and a potential V₄₁ is applied to the lower charge movement control electrode from the drive circuit, and charge is accumulated in the inorganic oxide semiconductor material layer (or the inorganic oxide semiconductor material layer and the photoelectric conversion layer, or the inorganic oxide semiconductor material layer, the protective layer, and the photoelectric conversion layer) in a charge accumulation period, and a potential V₁₂ is applied to the first electrode and a potential V₃₂ is applied to the charge accumulation electrode and a potential V₄₂ is applied to the lower charge movement control electrode from the drive circuit, and the charge accumulated in the inorganic oxide semiconductor material layer (or the inorganic oxide semiconductor material layer and the photoelectric conversion layer, or the inorganic oxide semiconductor material layer, the protective layer, and the photoelectric conversion layer) is read by the control unit via the first electrode in a charge transfer period.

Here, V₃₁>V₁₁, V₃₁>V₄₁, and V₁₂>V₃₂>V₄₂ are satisfied.

### [E09] <<Upper Charge Movement Control Electrode>

The imaging element according to any one of [E01] to [E08], in which an upper charge movement control electrode is formed on the region of the photoelectric conversion layer located between the adjacent imaging elements instead of formation of the second electrode.

[E10] The imaging element according to [E09], in which the second electrode is provided in each imaging element, and the upper charge movement control electrode is provided on the region of the photoelectric conversion layer that is separated from the second electrode with at least a part of the second electrode surrounded therewith.
[E11] The imaging element according to [E09], in which the second electrode is provided in each imaging element, and the upper charge movement control electrode is provided to be separated from the second electrode with at least a part of the second electrode surrounded therewith, and a part of the charge accumulation electrode is present below the upper charge movement control electrode.
[E12] The imaging element according to any one of [E09] to [E11], in which the second electrode is provided in each imaging element, the upper charge movement control electrode is provided to be separated from the second electrode with at least a part of the second electrode surrounded therewith, a part of the charge accumulation electrode is present below the upper charge movement control electrode, and further, the lower charge movement control electrode is formed below the upper charge movement control electrode.

### [E13] <<Control of Potentials of First Electrode, Charge Accumulation Electrode, and Charge Movement Control Electrode>>

The imaging element according to any one of [E09] to [E12], further including: a control unit that is provided in the semiconductor substrate and includes a drive circuit, in which the first electrode, the second electrode, the charge accumulation electrode, and the charge movement control electrode are connected to a drive circuit, a potential V₂₁ is applied to the second electrode and a potential V₄₁ is applied to the charge movement control electrode from the drive circuit, and charge is accumulated in the inorganic oxide semiconductor material layer (or the inorganic oxide semiconductor material layer and the photoelectric conversion layer, or the inorganic oxide semiconductor material layer, the protective layer, and the photoelectric conversion layer) in a charge accumulation period, and a potential V₂₂ is applied to the second electrode and a potential V₄₂ is applied to the charge movement control electrode from the drive circuit, and the charge accumulated in the inorganic oxide semiconductor material layer (or the inorganic oxide semiconductor material layer and the photoelectric conversion layer, or the inorganic oxide semiconductor material layer, the protective layer, and the photoelectric conversion layer) is read by the control unit via the first electrode in a charge transfer period.

Here, V₂₁>V₄₁ and V₂₂>V₄₂ are satisfied.

### [E14] <<Transfer Control Electrode>>

The imaging element according to any one of [E01] to [E13], further comprising: a transfer control electrode that is disposed to be separated from the first electrode and the charge accumulation electrode and to be disposed to face the inorganic oxide semiconductor material layer via an insulating layer, between the first electrode and the charge accumulation electrode.

### [E15] <<Control of Potentials of First Electrode, Charge Accumulation Electrode, and Transfer Control Electrode>>

The imaging element according to [E14], further including: a control unit that is provided in the semiconductor substrate and includes a drive circuit, in which the first electrode, the charge accumulation electrode, and the transfer control electrode are connected to a drive circuit, a potential V₁₁ is applied to the first electrode and a potential V₃₁ is applied to the charge accumulation electrode and a potential V₅₁ is applied to the transfer control electrode from the drive circuit, and charge is accumulated in the inorganic oxide semiconductor material layer (or the inorganic oxide semiconductor material layer and the photoelectric conversion layer, or the inorganic oxide semiconductor material layer, the protective layer, and the photoelectric conversion layer) in a charge accumulation period, and a potential V₁₂ is applied to the first electrode and a potential V₃₂ is applied to the charge accumulation electrode and a potential V₅₂ is applied to the transfer control electrode from the drive circuit, and the charge accumulated in the inorganic oxide semiconductor material layer (or the inorganic oxide semiconductor material layer and the photoelectric conversion layer, or the inorganic oxide semiconductor material layer, the protective layer, and the photoelectric conversion layer) is read by the control unit via the first electrode in a charge transfer period.

Here, the potential of the first electrode is higher than the potential of the second electrode, and V₃₁>V₅₁ and V₃₂≤V₅₂≤V₁₂ are satisfied.

### [E16] <<Charge Discharging Electrode>>

The imaging element according to any one of [E01] to [E15], further including: a charge discharging electrode that is connected to the inorganic oxide semiconductor material layer and is disposed to be separated from the first electrode and the charge accumulation electrode.

[E17] The imaging element according to [E16], in which the charge discharging electrode is disposed to surround the first electrode and the charge accumulation electrode.
[E18] The imaging element according to [E16] or [E17], in which the inorganic oxide semiconductor material layer (or the inorganic oxide semiconductor material layer and the protective layer) extends in a second opening portion provided in the insulating layer and is connected to the charge discharging electrode, an edge portion of a top surface of the charge discharging electrode is covered with the insulating layer, the charge discharging electrode is exposed from a bottom surface of the second opening portion, and when a surface of the insulating layer that is in contact with the top surface of the charge discharging electrode is defined as a third surface, and a surface of the insulating layer that is in contact with a portion of the inorganic oxide semiconductor material layer facing the charge accumulation electrode is defined as a second surface, a side surface of the second opening portion has an inclination spreading from the third surface toward the second surface.

### [E19] <<Control of Potential of First Electrode, Charge Accumulation Electrode, and Charge Discharging Electrode>>

The imaging element according to any one of [E16] to [E18], further including: a control unit that is provided in the semiconductor substrate and includes a drive circuit, in which the first electrode, the charge accumulation electrode, and the charge discharging electrode are connected to the drive circuit, a potential V₁₁ is applied to the first electrode and a potential V₃₁ is applied to the charge accumulation electrode and a potential V₆₁ is applied to the charge discharging electrode from the drive circuit, and charge is accumulated in the inorganic oxide semiconductor material layer (or the inorganic oxide semiconductor material layer and the photoelectric conversion layer, or the inorganic oxide semiconductor material layer, the protective layer, and the photoelectric conversion layer) in a charge accumulation period, and a potential V₁₂ is applied to the first electrode and a potential V₃₂ is applied to the charge accumulation electrode and a potential V₆₂ is applied to the charge discharging electrode from the drive circuit, and the charge accumulated in the inorganic oxide semiconductor material layer (or the inorganic oxide semiconductor material layer and the photoelectric conversion layer, or the inorganic oxide semiconductor material layer, the protective layer, and the photoelectric conversion layer) is read by the control unit via the first electrode in a charge transfer period.

Here, the potential of the first electrode is higher than the potential of the second electrode, V₆₁>V₁₁ and V₆₂<V₁₂ are satisfied.

### [E20] <<Charge Accumulation Electrode Segment>>

The imaging element according to any one of [E01] to [E19], in which the charge accumulation electrode is configured of a plurality of charge accumulation electrode segments.

[E21] The imaging element according to [E20], in which a potential applied to a charge accumulation electrode segment located at a position closest to the first electrode is higher than a potential applied to a charge accumulation electrode segment located at a position furthest from the first electrode in the charge transfer period in a case in which the potential of the first electrode is higher than the potential of the second electrode, and the potential applied to the charge accumulation electrode segment located at the position closest to the first electrode is lower than the potential applied to the charge accumulation electrode segment located at the position furthest from the first electrode in the charge transfer period in a case in which the potential of the first electrode is lower than the potential of the second electrode.
[E22] The imaging element according to any one of [E01] to [E21], in which the semiconductor substrate is provided with at least a floating diffusion layer and an amplification transistor configuring the control unit, and the first electrode is connected to the floating diffusion layer and a gate portion of the amplification transistor.
[E23] The imaging element according to [E22], in which the semiconductor substrate is further provided with a reset transistor and a selection transistor configuring the control unit, the floating diffusion layer is connected to one source/drain region of the reset transistor, one source/drain region of the amplification transistor is connected to one source/drain region of the selection transistor, and the other source/drain region of the selection transistor is connected to a signal line.
[E24] The imaging element according to any one of [E01] to [E23], in which a size of the charge accumulation electrode is larger than a size of the first electrode.
[E25] The imaging element according to any one of [E01] to [E24], in which light is incident from a side of the second electrode, and a light shielding layer is formed on a light incident side beyond the second electrode.
[E26] The imaging element according to any one of [E01] to [E24], in which light is incident from a side of the second electrode and no light is incident on the first electrode.
[E27] The imaging element according to [E26], in which a light shielding layer is formed on a light incident side beyond the second electrode and above the first electrode.
[E28] The imaging element according to [E26], in which an on-chip micro lens is provided above the charge accumulation electrode and the second electrode, and light that is incident on the on-chip micro lens is collected by the charge accumulation electrode.

### [E29] <<Imaging Element: First Configuration>>

The imaging element according to any one of [E01] to [E28], in which the photoelectric conversion unit is configured of N (where N>2) photoelectric conversion unit segments, the inorganic oxide semiconductor material layer and the photoelectric conversion layer (or the inorganic oxide semiconductor material layer, the protective layer, and the photoelectric conversion layer) are configured of N photoelectric conversion layer segments, the insulating layer is configured of N insulating layer segments, the charge accumulation electrode is configured of N charge accumulation electrode segments, an n-th (where n=1, 2, 3, . . . N) photoelectric conversion unit segment is configured of an n-th charge accumulation electrode segment, an n-th insulating layer segment, and an n-th photoelectric conversion layer segment, a photoelectric conversion segment with a larger n value is located further from the first electrode, and thicknesses of the insulating layer segments gradually change from the first photoelectric conversion unit segment to the N-th photoelectric conversion unit segment,

### [E30] <<Imaging Element: Second Configuration>>

The imaging element according to any one of [E01] to [E28], in which the photoelectric conversion unit is configured of N (where N>2) photoelectric conversion unit segments, the inorganic oxide semiconductor material layer and the photoelectric conversion layer (or the inorganic oxide semiconductor material layer, the protective layer, and the photoelectric conversion layer) are configured of N photoelectric conversion layer segments, the insulating layer is configured of N insulating layer segments, the charge accumulation electrode is configured of N charge accumulation electrode segments, n-th (where n=1, 2, 3, . . . , N) photoelectric conversion unit segment is configured of an n-th charge accumulation electrode segment, an n-th insulating layer segment, and an n-th photoelectric conversion layer segment, a photoelectric conversion unit segment with a larger n value is located further from the first electrode, and thicknesses of the photoelectric conversion layer segments gradually change from the first photoelectric conversion unit segment to the N-th photoelectric conversion unit segment.

### [E31] <<Imaging Element: Third Configuration>>

The imaging element according to any one of [E01] to [E28], in which the photoelectric conversion unit is configured of N (where N>2) photoelectric conversion unit segments, the inorganic oxide semiconductor material layer and the photoelectric conversion layer (or the inorganic oxide semiconductor material layer, the protective layer, and the photoelectric conversion layer) are configured of N photoelectric conversion layer segments, the insulating layer is configured of N insulating layer segments, the charge accumulation electrode is configured of N charge accumulation electrode segments, an n-th (where n=1, 2, 3, . . . , N) photoelectric conversion unit segment is configured of an n-th charge accumulation electrode segment, an n-th insulating layer segment, and an n-th photoelectric conversion layer segment, a photoelectric conversion unit segment with a larger n value is located further from the first electrode, and materials configuring the insulating layer segments are different in adjacent photoelectric conversion unit segments.

### [E32] <<Imaging Element: Fourth Configuration>>

The imaging element according to any one of [E01] to [E28], in which the photoelectric conversion unit is configured of N (where N>2) photoelectric conversion unit segments, the inorganic oxide semiconductor material layer and the photoelectrical conversion layer (or the inorganic oxide semiconductor material layer, the protective layer, and the photoelectric conversion layer) are configured of N photoelectric conversion layer segments, the insulating layer is configured of N insulating layer segments, the charge accumulation electrode is configured of N charge accumulation electrode segments disposed to be separated from each other, an n-th (where n=1, 2, 3, . . . , N) photoelectric conversion unit segment is configured of an n-th charge accumulation electrode segment, an n-th insulating layer segment, and an n-th photoelectric conversion layer segment, a photoelectric conversion unit segment with a larger n value is located at a further position from the first electrode, and materials configuring charge accumulation electrode segments are different in adjacent photoelectric conversion unit segments.

### [E33] <<Imaging Element: Fifth Configuration>>

The imaging element according to any one of [E01] to [E28], in which the photoelectric conversion unit is configured of N (where N>2) photoelectric conversion unit segments, the inorganic oxide semiconductor material layer and the photoelectric conversion layer (or the inorganic oxide semiconductor material layer, the protective layer, and the photoelectric conversion layer) are configured of N photoelectric conversion layer segments, the insulating layer is configured of N insulating layer segments, the charge accumulation electrode is configured of N charge accumulation electrode segments disposed to be separated from each other, an n-th (where n=1, 2, 3, . . . , N) photoelectric conversion unit segment is configured of an n-th charge accumulation electrode segment, an n-th insulating layer segment, and an n-th photoelectric conversion layer segment, a photoelectric conversion unit segment with a larger n value is located at a further position from the first electrode, and areas of the charge accumulation electrode segments gradually degrease from the first photoelectric conversion unit segment to the N-th photoelectric conversion unit segment.

### [E34] <<Imaging Element: Sixth Configuration>>

The imaging element according to any one of [E01] to [E28], in which when a lamination direction of the charge accumulation electrode, the insulating layer, the inorganic oxide semiconductor material layer, and the photoelectric conversion layer is defined as a Z direction, and a direction away from the first direction is defined as an X direction, a sectional area of a laminated portion in which the charge accumulation electrode, the insulating layer, the inorganic oxide semiconductor material layer, and the photoelectric conversion layer are laminated when the laminated portion is cut along a YZ virtual plane changes depending on a distance from the first electrode.

### [F01] <<Stacked Imaging Element>>

A stacked imaging element including: at least one imaging element according to any one of [A01] to [E34].

### [G01] <<Solid-State Imaging Device: First Aspect>>

A solid-state imaging device including: a plurality of imaging elements according to any one of [A01] to [E34].

### [G02] <<Solid-State Imaging Device: Second Aspect>

A solid-state imaging device including: a plurality of stacked imaging elements according to [F01].

### [H01] <<Solid-State Imaging Device: First Configuration>>

A solid-state imaging device including: a photoelectric conversion unit that is configured of a first electrode, a photoelectric conversion layer, and a second electrode being laminated, in which the photoelectric conversion unit includes a plurality of imaging elements according to any one of [A01] to [E34], the plurality of imaging elements configure an imaging element block, and the first electrode is shared by the plurality of imaging elements configuring the imaging element block.

### [H02] <<Solid-State Imaging Device: Second Configuration>>

A solid-state imaging device including: a plurality of stacked imaging elements according to [F01], in which the plurality of imaging elements configure an imaging element block, and a first electrode is shared by the plurality of imaging elements configuring the imaging element block.

[H03] The solid-state imaging device according to [H01] or [H02], in which one on-chip micro lens is disposed above one imaging element.
[H04] The solid state imaging device according to [H01] or [H02], in which two imaging elements configure the imaging element block, and one on-chip micro lens is disposed above the imaging element block.
[H05] The solid-state imaging device according to any one of [H01] to [H04], in which one floating diffusion layer is provided for the plurality of imaging elements.
[H06] The solid-state imaging device according to any one of [H01] to [H05], in which the first electrode is disposed to adjacent to the charge accumulation electrode of each imaging element.
[H07] The solid-state imaging device according to any one of [H01] to [H06], in which the first electrode is disposed to be adjacent to the charge accumulation electrodes of a part of the plurality of imaging elements and is not disposed to be adjacent to the charge accumulation electrodes of remaining ones of the plurality of imaging elements.
[H08] The solid-state imaging device according to [H07], in which a distance between the charge accumulation electrode configuring the imaging element and the charge accumulation electrode configuring the imaging element is longer than a distance between the first electrode and the charge accumulation electrode in the imaging element that is adjacent to the first electrode.

### [J01] <<Inorganic Oxide Semiconductor Material>>

An inorganic oxide semiconductor material with a composition represented as GaₐSn_{b}Zn_{c}O_{d} (where a+b+c=1.00, and a>0, b>0, and c>0), values of a, b, and c satisfy Expression (1) below, or satisfy Expression (2) below, or satisfy Expression (3) below, or satisfy Expressions (1) and (2) below, or satisfy Expressions (1) and (3) below, or satisfy Expressions (2) and (3) below, or satisfy Expressions (1), (2), and (3).

0.45(b−0.62)≤0.55a≤0.45b (1)

a≤−3.0(b−0.63) (2)

b≥0.23 (3)

[J02] The inorganic oxide semiconductor material according to [J01], in which carrier concentration is equal to or greater than 1×10¹⁴ cm⁻³ and equal to or less than 1×10¹⁷ cm⁻³.
[J03] The inorganic oxide semiconductor material according to [J01] or [J02], in which carrier mobility is equal to or greater than 10 cm²/V·s,
[J04] The inorganic oxide semiconductor material according to any one of [J01] to [J03], in which an optical gap of the inorganic oxide semiconductor material is equal to or greater than 2.7 eV and equal to or less than 3.2 eV.
[J05] The inorganic oxide semiconductor material according to any one of [J01] to [J03], in which oxygen deficiency generation energy of the inorganic oxide semiconductor material is equal to or greater than 2.6 eV.

### [K01] <<Method for Driving Solid-State Imaging Device>>

A method for driving a solid-state imaging device including a plurality of imaging elements, each of which includes a photoelectric conversion unit that is configured of a first electrode, a photoelectric conversion layer, and a second electrode being laminated, in each of which the photoelectric conversion unit further includes a charge accumulation electrode disposed to be separated from the first electrode and disposed to face the photoelectric conversion layer via an insulating layer, and each of which has a structure in which light is incident from a side of the second electrode and no light is incident on the first electrode, the method including: repeating each of the processes of discharging charge at the first electrodes to outside of a system while accumulating the charge in the inorganic oxide semiconductor material layers at once in all the imaging elements, then transferring the charge accumulated in the inorganic oxide semiconductor material layers to the first electrodes at once in all the imaging elements, and after the transfer is completed, successively reading the charge transferred to the first electrode in each imaging element.

### [L01] <<Method for Manufacturing Imaging Element>>

A method for manufacturing an imaging element in which an inorganic oxide semiconductor material layer, and a photoelectric conversion layer made of organic materials and a second electrode are successively formed on an underlayer where a first electrode is formed, the method including: after forming the inorganic oxide semiconductor material layer, performing annealing at 250° C. or less in an atmosphere containing water vapor.

### [L02] <<Method for Manufacturing Imaging Element>>

A method for manufacturing an imaging element including a photoelectric conversion unit that is configured of a first electrode, and a photoelectric conversion layer made of organic materials and a second electrode being laminated, an inorganic oxide semiconductor material layer made of a first layer and a second layer from a side of the first electrode being formed between the first electrode and the photoelectric conversion layer, the method including: forming the first layer on the basis of a sputtering method; and then forming the second layer on the basis of the sputtering method with smaller input power than input power for forming the first layer.

### REFERENCE SIGNS LIST

10 Imaging element (stacked imaging element, first imaging element)
11 Second imaging element
12 Third imaging element
13 Various imaging element components located below interlayer insulating layer
14 On-chip micro lens (OCL)
15 Light shielding layer
21 First electrode
22 Second electrode
23 Photoelectric conversion laminated body
23A Photoelectric conversion layer
23B Inorganic oxide semiconductor material layer
24 Charge accumulation electrode
24A, 24B, 24C Charge accumulation electrode segment
25, 25A, 25B Transfer control electrode (charge transfer electrode)
26 Charge discharging electrode
27 Lower charge movement control electrode (lower-side charge movement control electrode)
27A Connection hole
27B Pad portion
28 Upper charge movement control electrode (upper-side charge movement control electrode)
41 n-type semiconductor region configuring second imaging element
43 n-type semiconductor region configuring third imaging element
42, 44, 73 p+ layer
45, 46 Gate portion of transfer transistor
51 Gate portion of reset transistor TR1ᵣₛₜ
51A Channel formation region of reset transistor TR1ᵣₛₜ
51B, 51C Source/drain region of reset transistor TR1ᵣₛₜ
52 Gate portion of amplification transistor TR1ₐₘₚ
52A Channel formation region of amplification transistor TR1ₐₘₚ
52B, 52C Source/drain region of amplification transistor TR1ₐₘₚ
53 Gate portion of selection transistor TR1ₛₑₗ
53A Channel formation region of selection transistor TR1ₛₑₗ
53B, 53C Source/drain region of selection transistor TR1ₛₑₗ
61 Contact hole portion
62 Wiring layer
63, 64, 68A Pad portion
65, 68B Connection hole
66, 67, 69 Connecting portion
70 Semiconductor substrate
70A First surface (front surface) of semiconductor substrate
70B Second surface (rear surface) of semiconductor substrate
71 Element separation region
72 Oxide film
74 HfO₂ film
75 Insulating material film
76, 81 Interlayer insulating layer
82 Insulating layer
82_{A} Region (region-a) between adjacent imaging elements
83 Protective material layer
84 Opening portion
85 Second opening portion
100 Solid-state imaging device
101 Stacked imaging element
111 Imaging region
112 Vertical drive circuit
113 Column signal processing circuit
114 Horizontal drive circuit
115 Output circuit
116 Drive control circuit
117 Signal line (data output line)
118 Horizontal signal line
200 Electronic equipment (camera)
201 Solid-state imaging device
210 Optical lens
211 Shutter device
212 Drive circuit
213 Signal processing circuit
FD₁, FD₂, FD₃, 45C, 46C Floating diffusion layer
TR1ₜᵣₛ, TR2ₜᵣₛ, TR3ₜᵣₛ Transfer transistor
TR1ᵣₛₜ, TR2ᵣₛₜ, TR3ᵣₛₜ Reset transistor
TR1ₐₘₚ, TR2ₐₘₚ, TR3ₐₘₚ Amplification transistor
TR1ₛₑₗ, TR3ₛₑₗ. TR3ₛₑₗ Selection transistor
V_{DD} Power source
RST₁, RST₂, RST₃ Reset line
SEL₁, SEL₂, SEL₃ Selection line
117, VSL, VSL₁, VSL₂, VSL₃ Signal line (data output line)
TG₂, TG₃ Transfer gate line
V_{OA}, V_{OB}, V_{OT}, V_{OU} Wiring

1. An imaging element comprising:
a photoelectric conversion unit that is configured of a first electrode and a photoelectric conversion layer including an organic material and a second electrode being laminated,
wherein an inorganic oxide semiconductor material layer is formed between the first electrode and the photoelectric conversion layer, and an inorganic oxide semiconductor material configuring the inorganic oxide semiconductor material layer contains gallium atoms, tin atoms, zinc atoms, and oxygen atoms.

2. The imaging element according to claim 1, wherein an optical gap of the inorganic oxide semiconductor material is equal to or greater than 2.7 eV and equal to or less than 3.2 eV.

3. The imaging element according to claim 2, wherein when a composition of the inorganic oxide semiconductor material is represented as GaaSnbZncOd (where a+b+c=1.00, and a>0, b>0, and c>0), 0.45(b−0.62) 0.55a≤0.45b . . . (1) is satisfied.

4. The imaging element according to claim 1, wherein oxygen deficiency generation energy of the inorganic oxide semiconductor material is equal to or greater than 2.6 eV.

5. The imaging element according to claim 4, wherein when a composition of the inorganic oxide semiconductor material is represented as GaaSnbZncOd (where a+b+c=1.00, and a>0, b>0, and c>0), a−3.0(b−0.63) . . . (2) is satisfied.

6. The imaging element according to claim 1, wherein a carrier mobility of the inorganic oxide semiconductor material layer is equal to or greater than 10 cm2/V·s, and when a composition of the inorganic oxide semiconductor material is represented as GaaSnbZncOd (where a+b+c=1.00, and a>0, b>0, and c>0), b 0.23 . . . (3) is satisfied.

7. The Imaging element according to claim 1,
wherein when a composition of the inorganic oxide semiconductor material is represented as GaaSnbZncOd (where a+b+c=1.00, and a>0, b>0, and c>0), values of a, b, and c
satisfy Expression (1) below, or
satisfy Expression (2) below, or
satisfy Expression (3) below, or
satisfy Expressions (1) and (2) below, or
satisfy Expressions (1) and (3) below, or
satisfy Expressions (2) and (3) below, or
satisfy Expressions (1), (2), and (3) below,
here,
0.45(b−0.62)≤0.55a≤0.45b (1)
a≤−3.0(b−0.63) (2)
b≥0.23 (3).

8. The imaging element according to claim 1, wherein when a composition of the inorganic oxide semiconductor material is represented as GaaSnbZncOd (where a+b+c=1.00, and a>0, b>0, and c>0), values of a, b, and c satisfy all of Expressions (1), (2), and (3) below:
0.45(b−0.62)≤0.55a≤0.45b (1)
a≤−3.0(b−0.63) (2)
b≥0.23 (3).

9. The imaging element according to claim 1, wherein carrier mobility of the inorganic oxide semiconductor material layer is equal to or greater than 10 cm2/V·s.

10. The imaging element according to claim 1, wherein the photoelectric conversion unit further includes an insulating layer and a charge accumulation electrode that is disposed to be separated from the first electrode and is disposed to face the inorganic oxide semiconductor material layer via the insulating layer.

11. The imaging element according to claim 1, wherein charge generated in the photoelectric conversion layer is transferred to the first electrode via the inorganic oxide semiconductor material layer.

12. The imaging element according to claim 11, wherein the charge is electrons.

13. A stacked imaging element comprising:
at least one imaging element according to claim 1.

14. A solid-state imaging device comprising:
a plurality of imaging elements according to claim 1.

15. A solid-state imaging device comprising:
a plurality of stacked imaging elements according to claim 13.

16. An inorganic oxide semiconductor material,
wherein when a composition is represented as GaaSnbZncOd (where a+b+c=1.00, and a>0, b>0, and c>0),
values of a, b, and c
satisfy Expression (1) below, or
satisfy Expression (2) below, or
satisfy Expression (3) below, or
satisfy Expressions (1) and (2) below, or
satisfy Expressions (1) and (3) below, or
satisfy Expressions (2) and (3) below, or
satisfy Expressions (1), (2), and (3) below,
0.45(b−0.62)≤0.55a≤0.45b (1)
a≤−3.0(b−0.63) (2)
b≥0.23 (3)
